(19) United States
(12) Patent Application Publication
(10) Pub. No.: US 2004/0259853 A1
(43) Pub. Date: Dec. 23, 2004

(54) Methods for preventing acute clinical vascular events in a subject

(75) Inventor: Ira Tabas, New City, NY (US)

Correspondence Address: John P. White, Esq., Cooper & Dunham LLP, 1185 Avenue of the Americas, New York, NY, 10036, US

(73) Assignee: The Trustees of Columbia University, New York, NY

(21) Appl. No.: 10/767,749

(22) Filed: Jan. 28, 2004

Related U.S. Application Data
(63) Continuation-in-part of application No. 09/553,927, filed on Apr. 21, 2000, now abandoned.
(63) Continuation-in-part of application No. 10/426,415, filed on Apr. 30, 2003.
(60) Provisional application No. 60/376,984, filed on Apr. 30, 2002.
(60) Provisional application No. 60/494,721, filed on Aug. 11, 2003.

(51) Int. Cl. A61K 31/57

(52) U.S. Cl. 514/178

(57) ABSTRACT

This invention provides methods for: (i) inhibiting macrophage death, (ii) inhibiting atherosclerotic lesional complications, and (iii) inhibiting necrosis, plaque rupture and/or superficial erosion, in a subject having, or at increased risk for developing, cardiovascular disease which comprise administering to the subject an effective amount of an amphiphilic compound or a pharmaceutically acceptable salt thereof which (i) inhibits the intracellular transport of cholesterol within cells or (ii) inhibits free cholesterol-induced death of cells, wherein the transport is from an intracellular cholesterol storage site to the endoplasmic reticulum. This invention also provides articles of manufacture comprising an amphiphilic compound which inhibits the intracellular transport of cholesterol from an intracellular cholesterol storage site to the endoplasmic reticulum in cells, for use in inhibiting (i) macrophage death, (ii) atherosclerotic lesional complications, or (iii) necrosis, plaque rupture and/or superficial erosion, in a subject having, or at increased risk for developing, cardiovascular disease.

This invention is a continuation-in-part and claims priority of U.S. Ser. No. 09/553,927, filed Apr. 21, 2000, U.S. Ser. No. 10/426,415, filed Apr. 30, 2003, which claims priority of U.S. Provisional Application No. 60/376,984, filed Apr. 30, 2002, and U.S. Provisional Application No. 60/494,721, filed Aug. 11, 2003, the contents of which are hereby incorporated by reference into this application.

The invention disclosed herein was made with United States government support under grant numbers HL54591, HL56984, HL61814, DK47119 and ES08681 from the National Institutes of Health. Accordingly, the United States government has certain rights in this invention.

Throughout this application, various publications are referenced by arabic numbers within parentheses. Full citations for these publications may be found at the end of each series of experiments in the specification. The disclosures of these publications are hereby incorporated by reference into this application to describe more fully the art to which this invention pertains.

### BACKGROUND OF THE INVENTION

A prominent feature of advanced atherosclerotic lesions is the presence of necrotic areas, which are sites inside the thickened intima consisting of cellular debris and extracellular lipid (1,2). The importance of these necrotic areas lies in the fact that they are often found in areas of plaque rupture, which is the most common precipitating cause of atherosclerosis-associated acute thrombosis, vascular occlusion, and tissue infarction (3).

Given the potential clinical implications of lesion necrosis, surprisingly little is known about the mechanisms of necrotic area development. While there is evidence that some of the lipid in these areas is derived directly from extracellular, plasma-derived lipoproteins, cell death with subsequent release of intracellular lipids and other potentially harmful molecules is likely to be a central event (2,4,5). In this regard, recent data with antibodies against cell-type-specific intracellular proteins support the idea that the cholesterol-loaded macrophage, a major cellular constituent of atherosclerotic lesions, is the main cell type that dies in the vicinity of necrotic areas (4,6). The mechanistic link between macrophage death and unstable plaques may be related to plaque-destabilizing enzymes and pro-coagulant/thrombogenic molecules released by these dying cells (7).

The causes of macrophage death in advanced atherosclerosis are not known. Several factors or conditions, such as oxidized lipids, growth factor deprivation, and inflammatory cytokines, have been proposed but not rigorously tested in vivo (1,8). Another cytotoxic condition that deserves attention is excess cellular free cholesterol (FC) (9). FC accumulation in lesional foam cells has been well-documented (10,11,12,13), and studies with cultured macrophages have shown that excess cellular FC is a potent inducer of cell death (14,15). The mechanism of cytotoxicity probably involves integral membrane protein dysfunction resulting from a high cholesterol:phospholipid ratio in the membranes surrounding these molecules (9,16,17). This idea was recently supported by the data of Kellner-Weibel et al. (18), who showed that FC-induced cytotoxicity was inhibited by amphipathic amines, which block the transport of lipoprotein-derived FC from lysosomes to peripheral membranes, particularly the plasma membrane.

Interestingly, Papahadjopoulos (16) demonstrated twenty-five years ago that FC-mediated inhibition of the plasma membrane proteins Na⁺-K⁺-ATPase and adenylate cyclase leads to cellular death, and he proposed that these events may play an important role in the development of necrosis in advanced atheromata.

Although cell cultures studies have suggested potentially important ideas related to inducers and mechanisms of macrophage death, little is known about the factors that influence the development of lesional necrosis in vivo.

It has been shown that free cholesterol loading causes macrophage death (14). Kellner-Weibel et al. (18) showed that progesterone and an amphipathic amine stop macrophage death in vitro.

One way in which this invention differs from the prior art is that it is the first study using molecular genetics and showing the effect on lesional necrosis in vivo. This invention therefore provides an advantage over the prior art in that it provides evidence that lesional necrosis can be decreased in vivo.

### SUMMARY OF THE INVENTION

This invention provides a method for inhibiting macrophage death in a subject having, or at increased risk for developing, cardiovascular disease which comprises administering to the subject an effective amount of an amphiphilic compound or a pharmaceutically acceptable salt thereof which inhibits the intracellular transport of cholesterol within cells, wherein the transport is from an intracellular cholesterol storage site to the endoplasmic reticulum, so as to thereby inhibit macrophage death in the subject.

This invention also provides a method for inhibiting atherosclerotic lesional complications in a subject having, or at increased risk for developing, cardiovascular disease which comprises administering to the subject an effective amount of an amphiphilic compound or a pharmaceutically acceptable salt thereof which inhibits the intracellular transport of cholesterol within cells, wherein the transport is from an intracellular cholesterol storage site to the endoplasmic reticulum, so as to thereby inhibit atherosclerotic lesional complications in the subject.

This invention also provides a method for inhibiting macrophage death in a subject having, or at increased risk for developing, cardiovascular disease which comprises administering to the subject an effective amount of an amphiphilic compound or a pharmaceutically acceptable salt thereof which inhibits free cholesterol-induced death of cells in the subject by inhibiting intracellular transport of cholesterol within the cells, wherein the transport is from an intracellular cholesterol storage site to the endoplasmic reticulum, so as to thereby inhibit macrophage death in the subject.

This invention also provides a method for inhibiting atherosclerotic lesional complications in a subject having, or at increased risk for developing, cardiovascular disease which comprises administering to the subject an effective amount of an amphiphilic compound or a pharmaceutically acceptable salt thereof which inhibits free cholesterol-induced death of cells in the subject by inhibiting intracellular transport of cholesterol within the cells, wherein the transport is from an intracellular cholesterol storage site to the endoplasmic reticulum, so as to thereby inhibit atherosclerotic lesional complications in the subject.

This invention further provides a method for inhibiting necrosis, plaque rupture and/or superficial erosion in a subject having, or at increased risk for developing, cardiovascular disease which comprises administering to the subject an effective amount of an amphiphilic compound or a pharmaceutically acceptable salt thereof which inhibits intracellular transport of cholesterol within cells, wherein the transport is from an intracellular cholesterol storage site to the endoplasmic reticulum, so as to thereby inhibit necrosis, plaque rupture and/or superficial erosion in the subject.

This invention provides an article of manufacture comprising packaging material and an amphiphilic compound, wherein the compound inhibits the intracellular transport of cholesterol from an intracellular cholesterol storage site to the endoplasmic reticulum in cells, and the packaging material comprises a label indicating that the compound is intended for use in inhibiting macrophage death in a subject having, or at increased risk for developing, cardiovascular disease.

This invention also provides an article of manufacture comprising packaging material and an amphiphilic compound, wherein the compound inhibits the intracellular transport of cholesterol from an intracellular cholesterol storage site to the endoplasmic reticulum in cells, and the packaging material comprises a label indicating that the compound is intended for use in inhibiting atherosclerotic lesional complications in a subject having, or at increased risk for developing, cardiovascular disease.

Finally, this invention provides an article of manufacture comprising packaging material and an amphiphilic compound, wherein the compound inhibits the intracellular transport of cholesterol within cells, wherein the transport is from an intracellular cholesterol storage site to the endoplasmic reticulum, wherein the packaging material comprises a label indicating that the compound is intended for use in inhibiting necrosis, plaque rupture and/or superficial erosion in a subject having, or at increased risk for developing cardiovascular disease.

### BRIEF DESCRIPTION OF THE FIGURES

FIG. 1

This figure shows that NPC1 macrophages are resistant to FC-mediated cytotoxicity. Peritoneal macrophages from wild-type and NPC1 mice were incubated for 24 h in serum-free medium alone (cross-hatched bars) or medium containing 10 μg acetyl-LDL/ml plus 10 μg 58035/ml (solid bars) to effect FC loading. The cells were then stained with propidium iodide- to determine the percentage of necrotic cells. Legend: cross-hatched bars=control; solid bars =FC-loaded.

FIGS. 2A, 2B, 2C and 2D

These figures show the characterization of necrotic areas in the advanced atherosclerotic lesions of E0 mice. Adjacent sections of proximal aortic lesions from 25-week-old cholesterol fed E0 mice were stained with hematoxylin (FIG. 2A), filipin (FIG. 2B), anti-type A scavenger receptor antibody (FIG. 2C), and control antibody (FIG. 2D). The arrow in each figure depicts one of the several sites that have the characteristics of necrotic areas.

FIGS. 3A and 3B

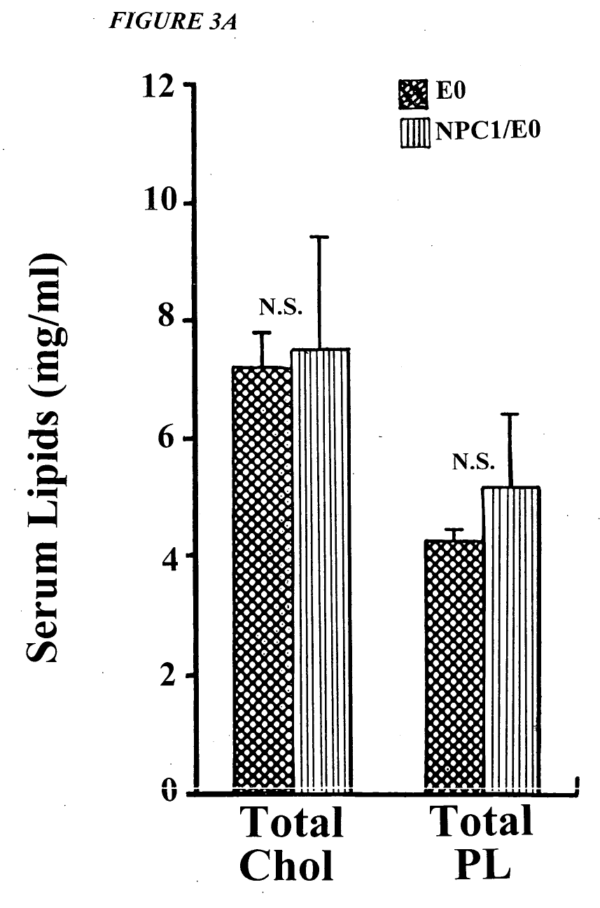

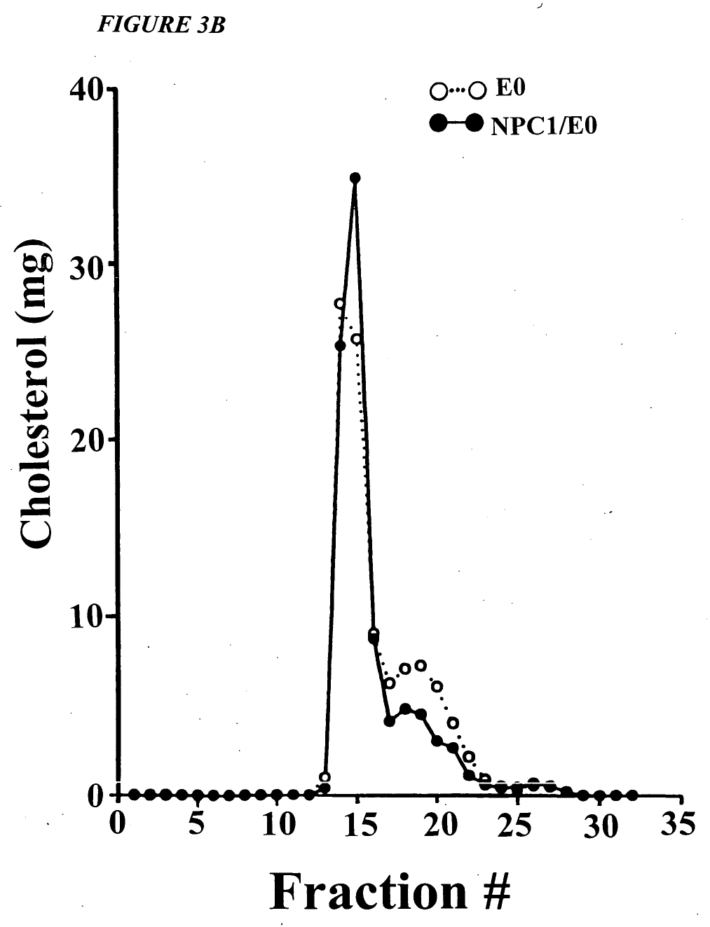

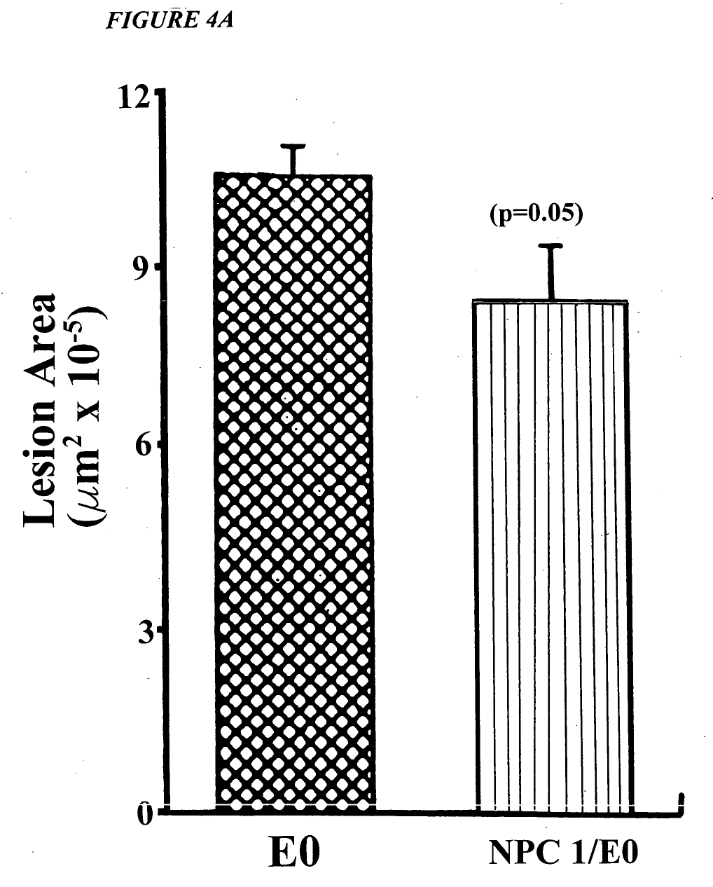

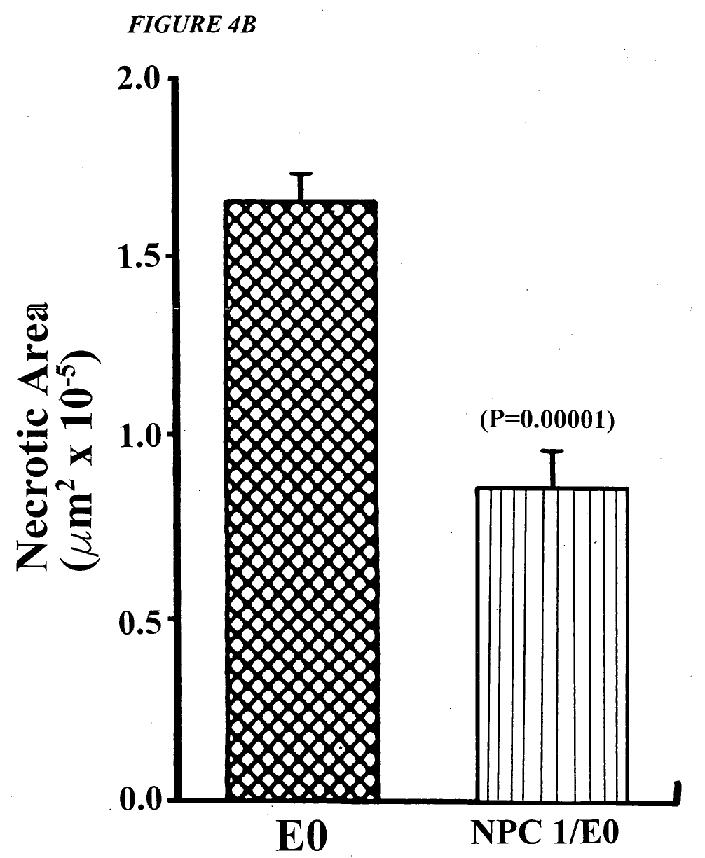

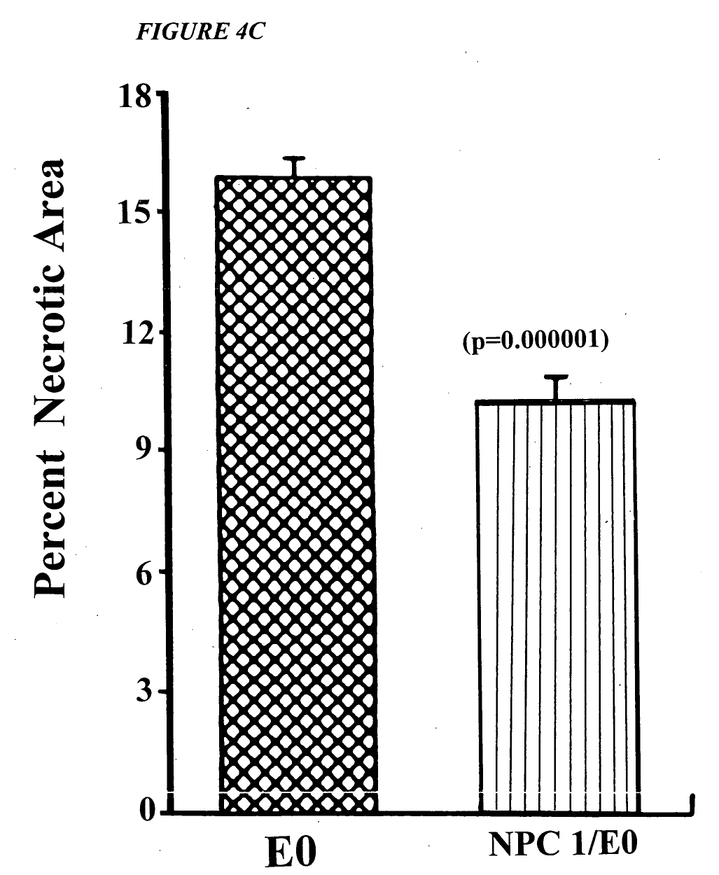

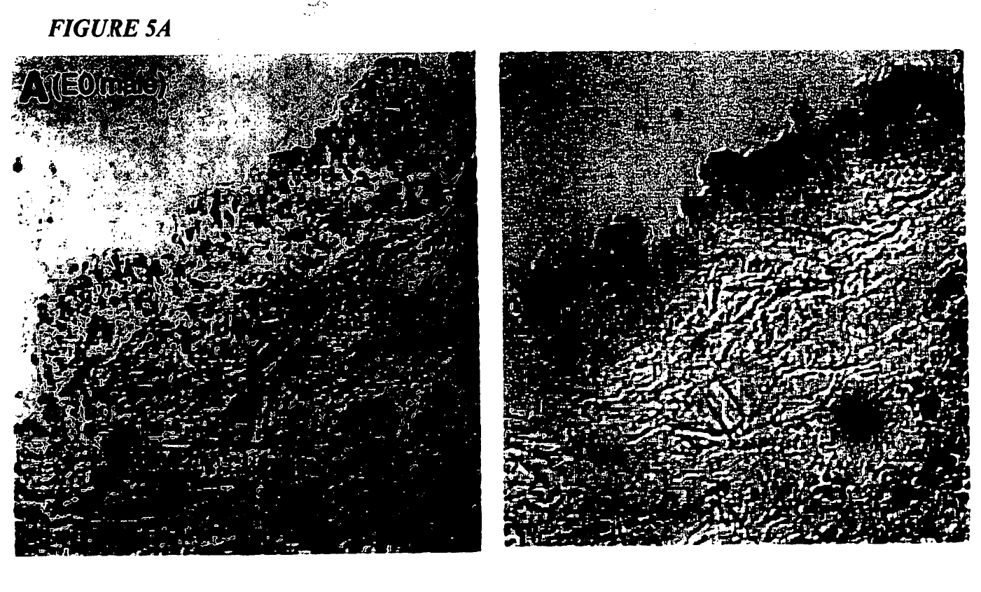

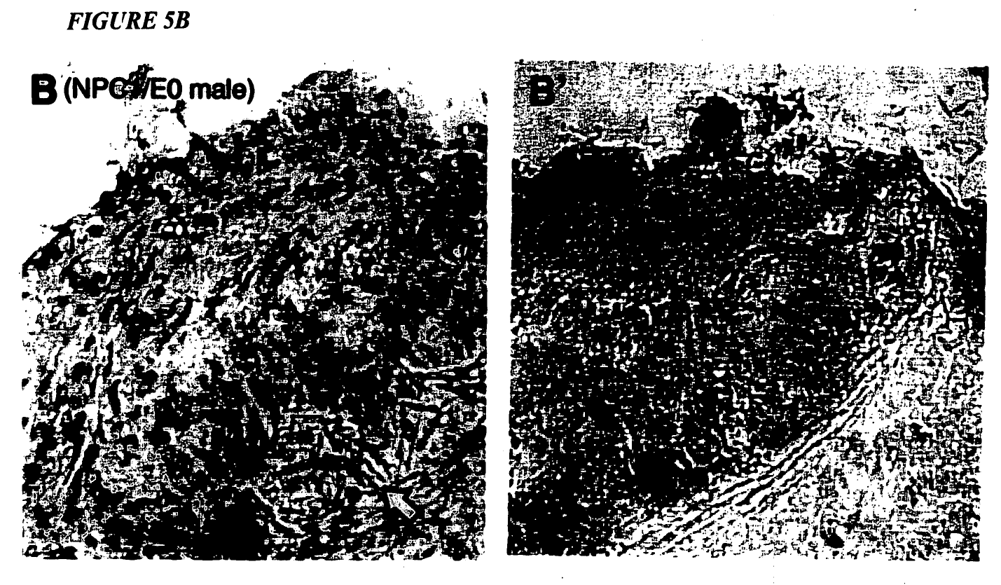

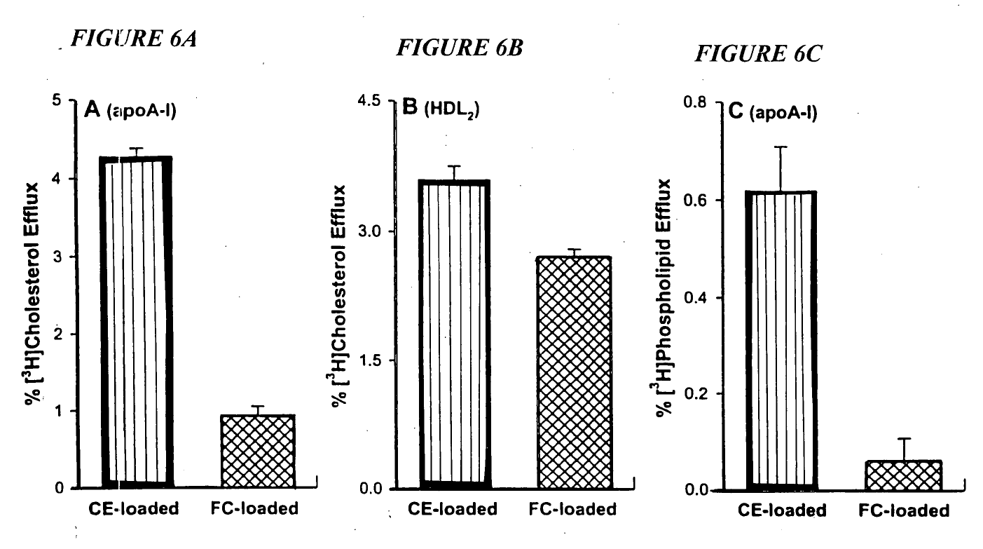

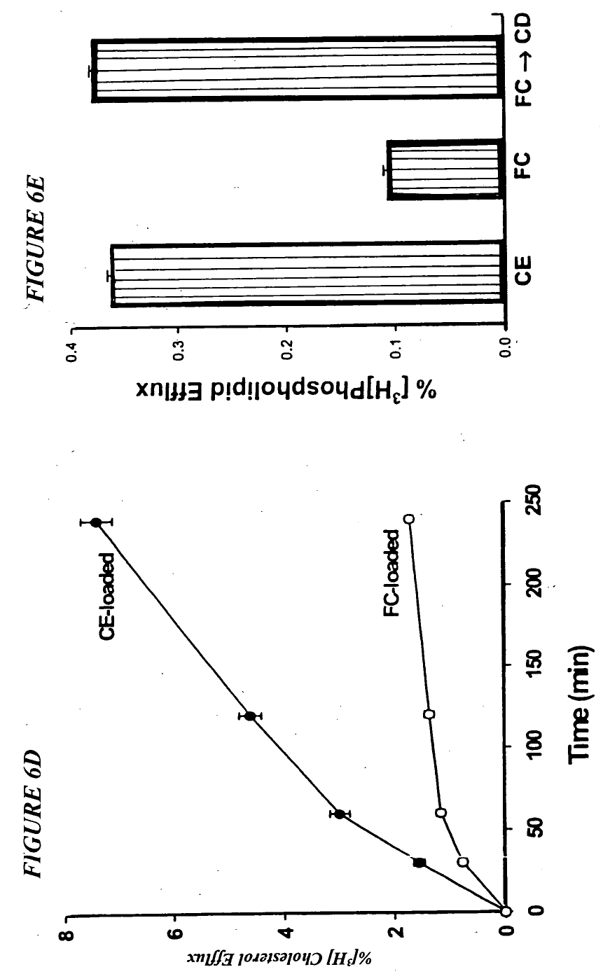

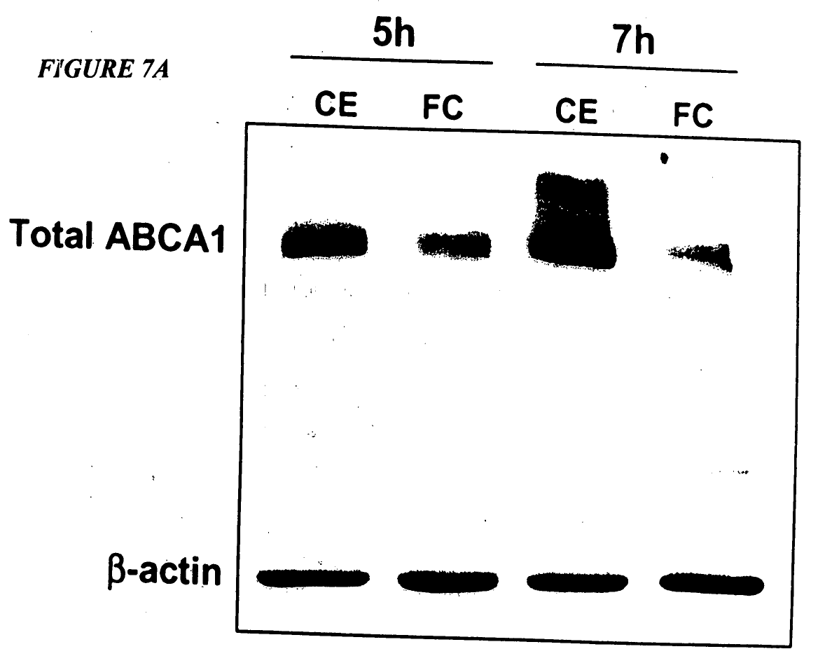

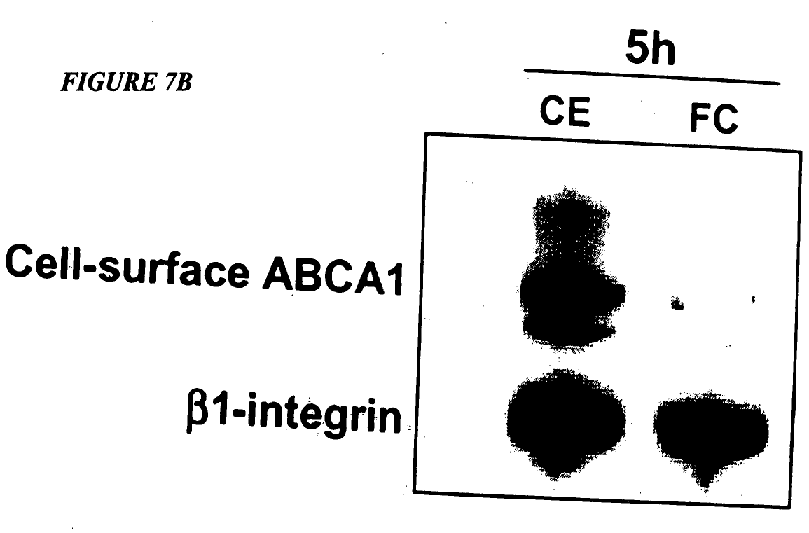

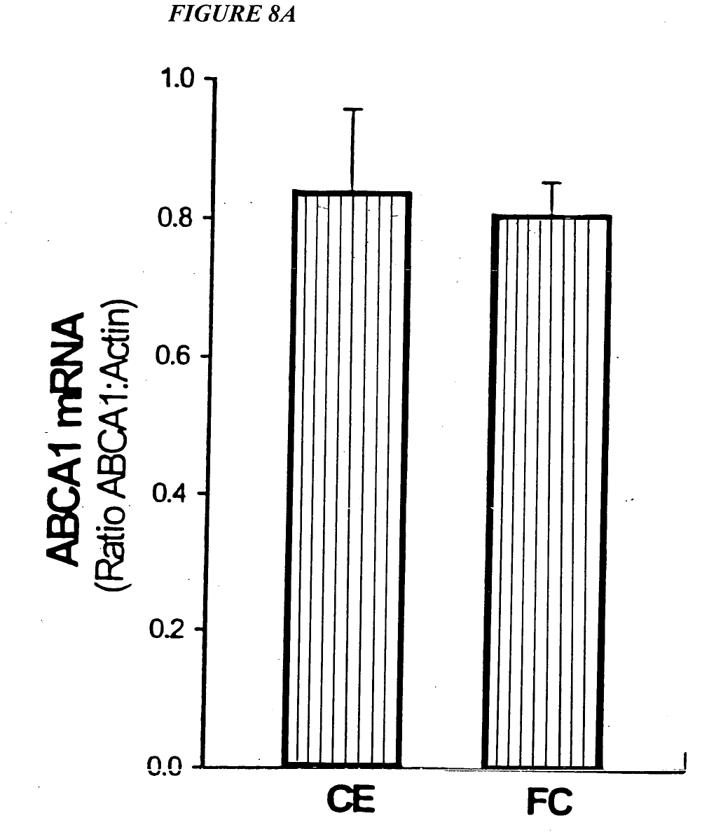

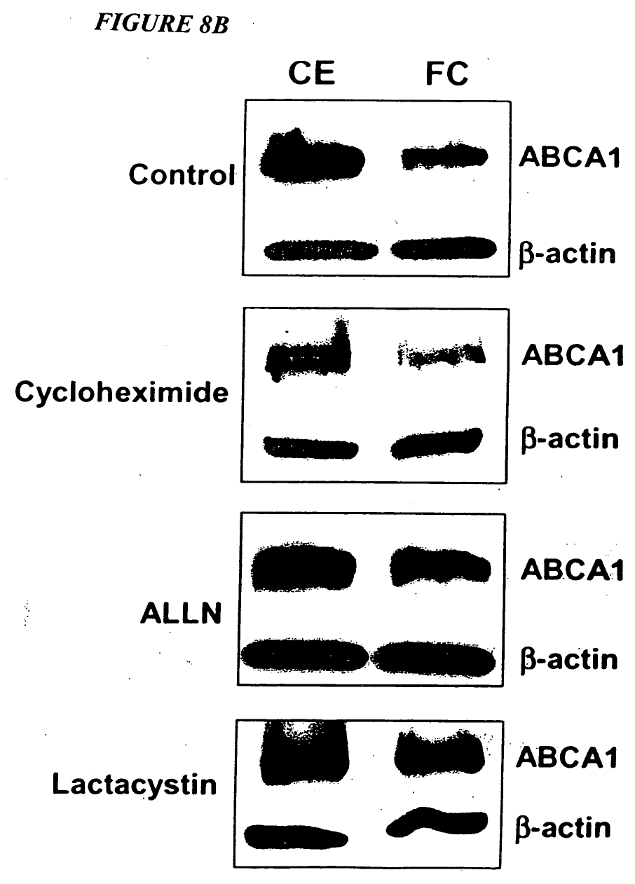

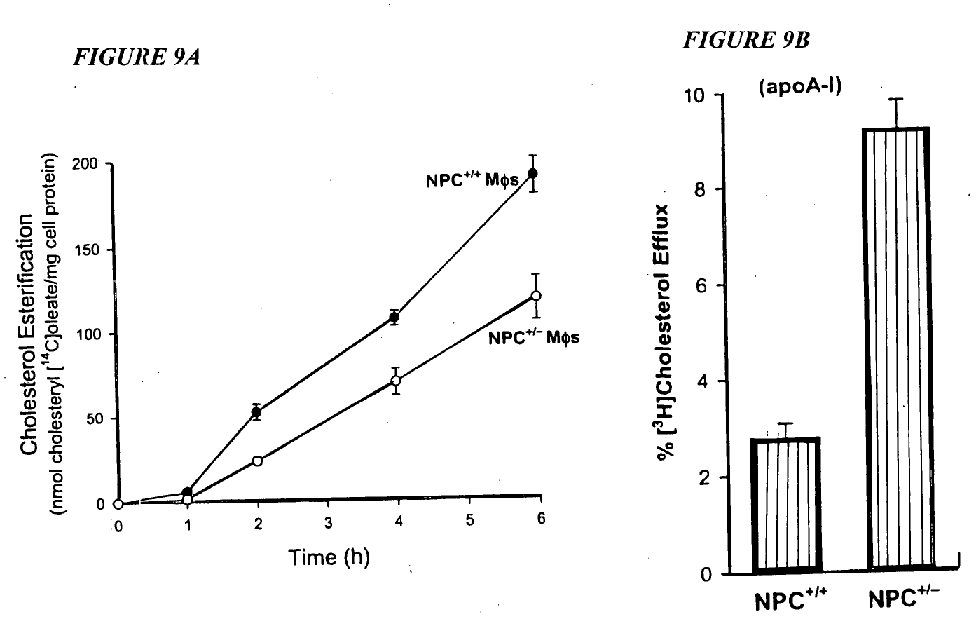

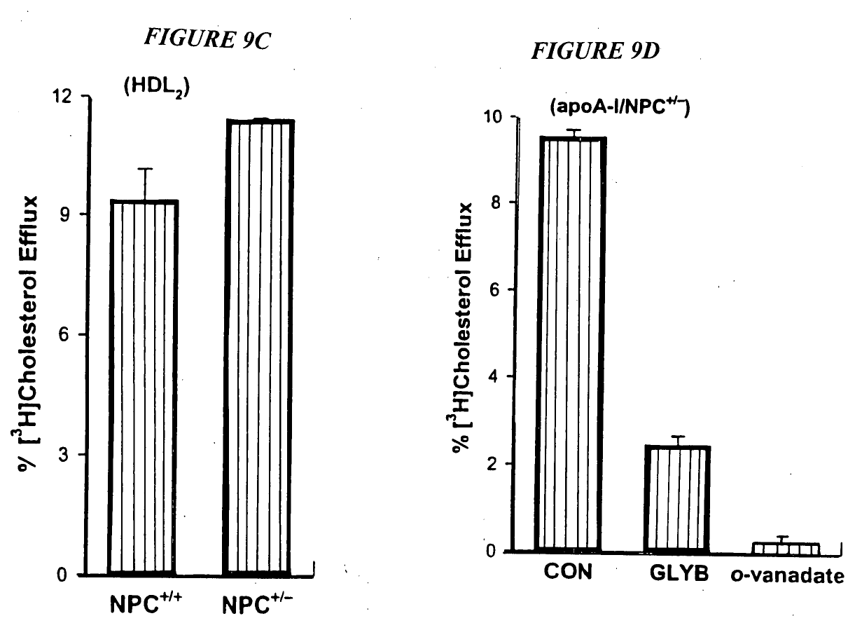

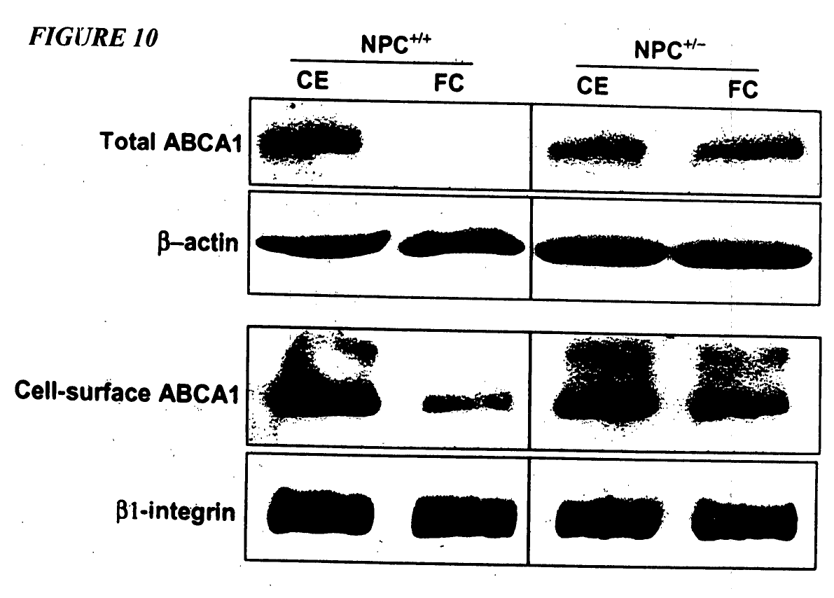

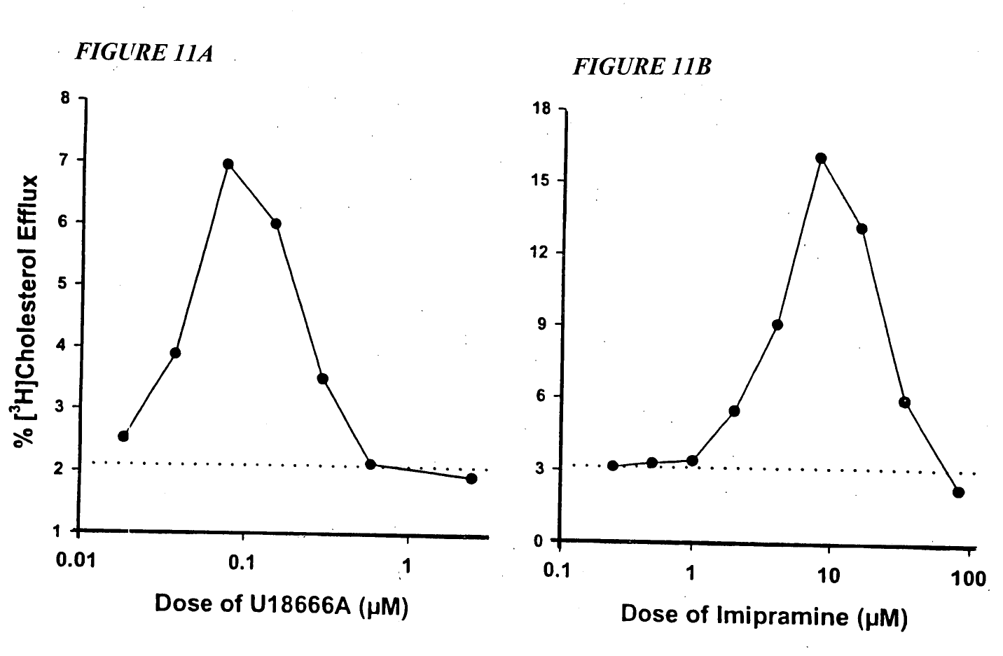

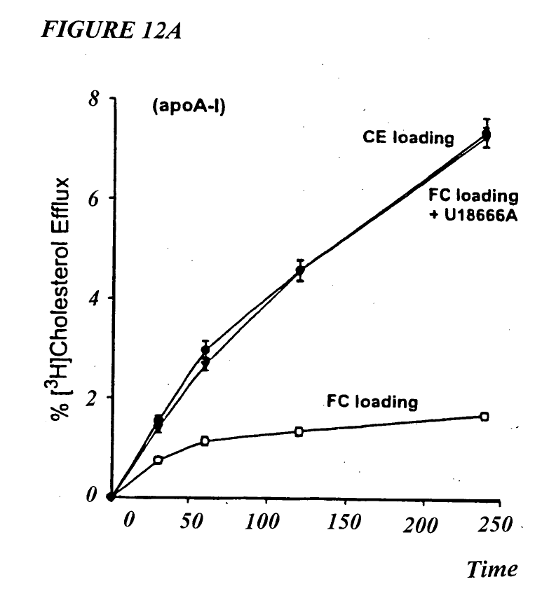

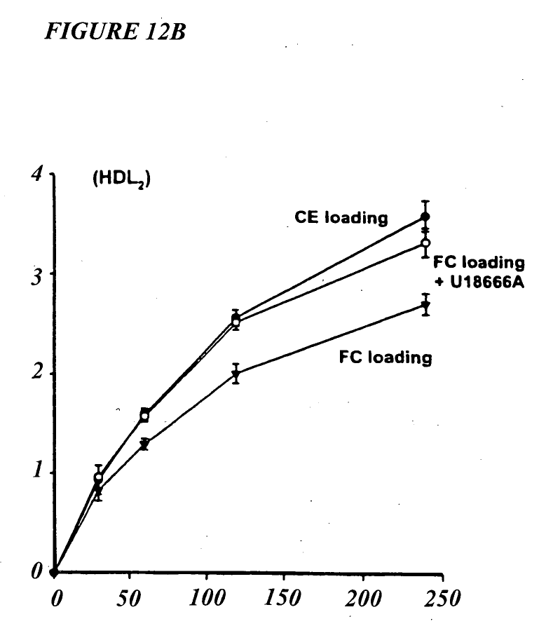

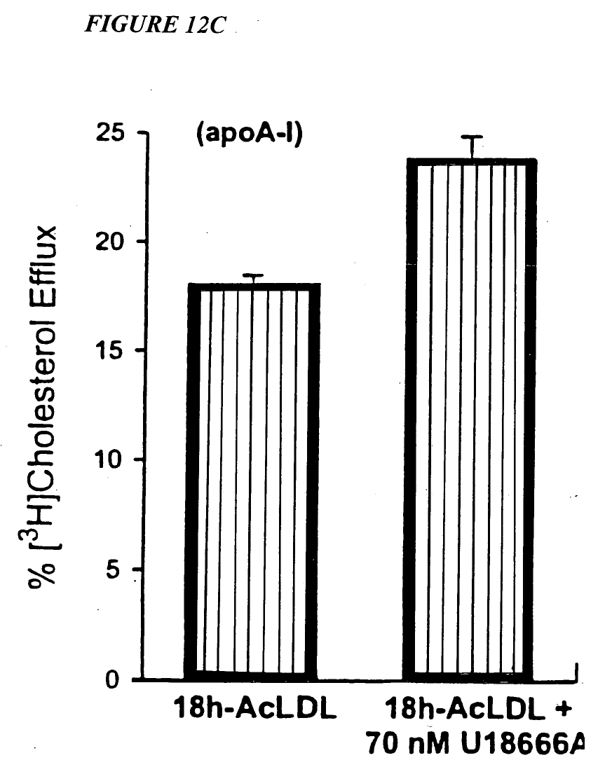

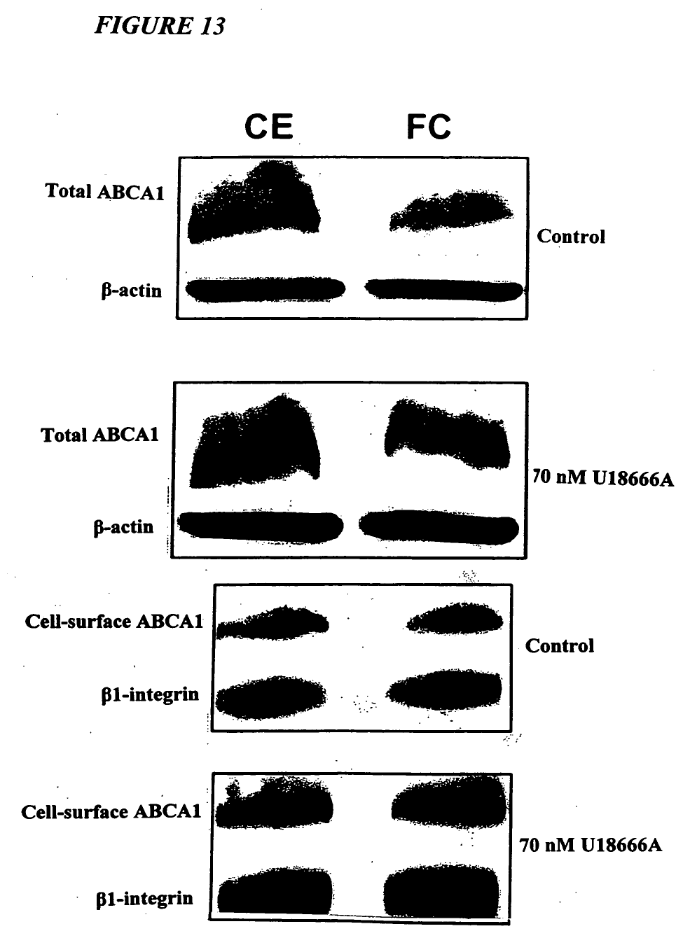

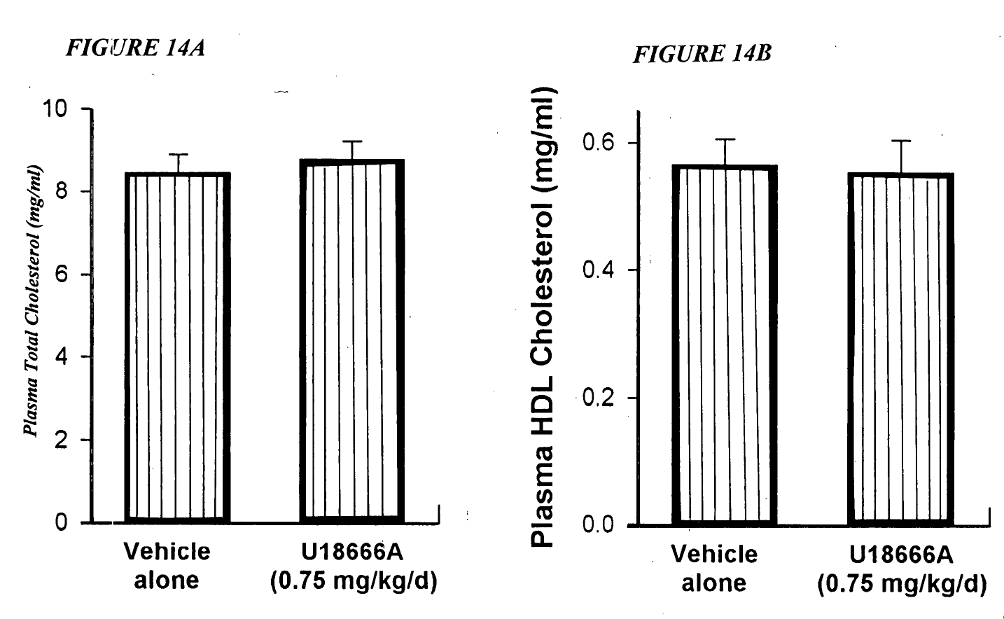

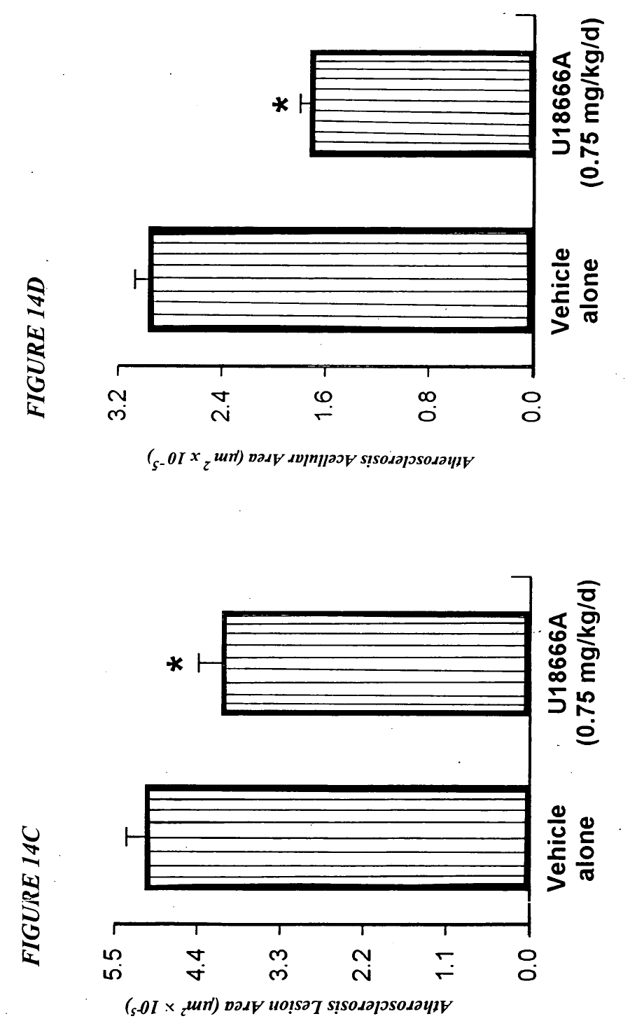

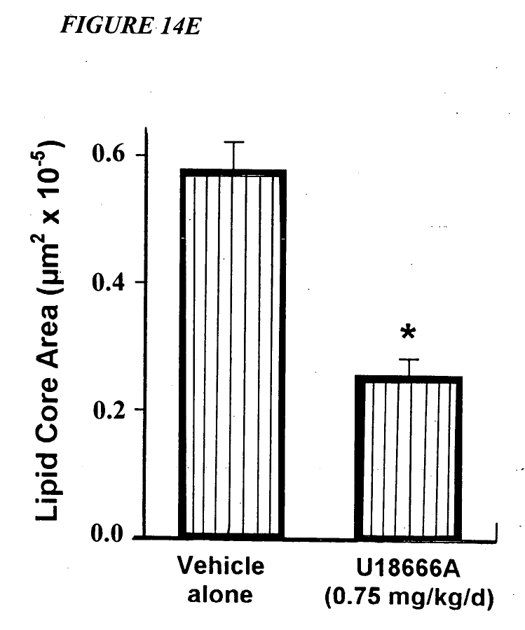

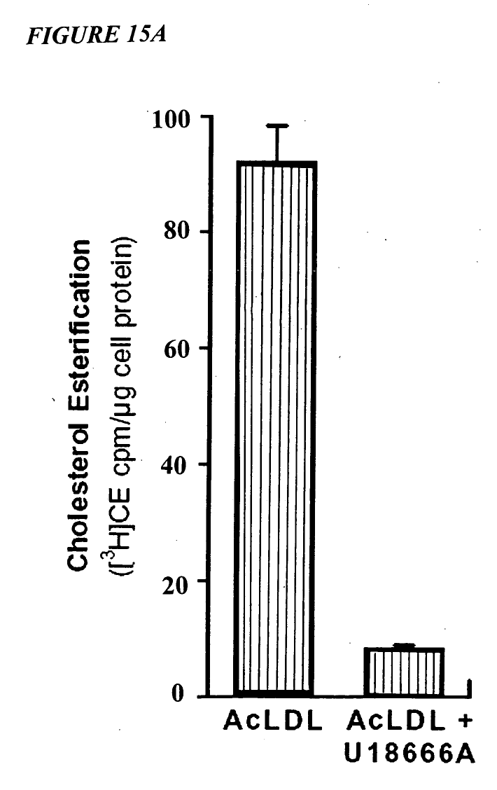

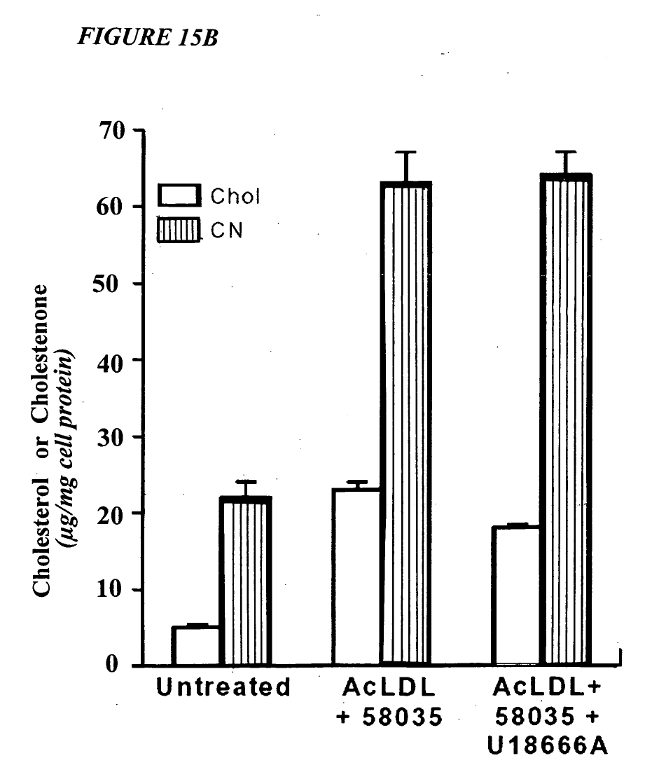

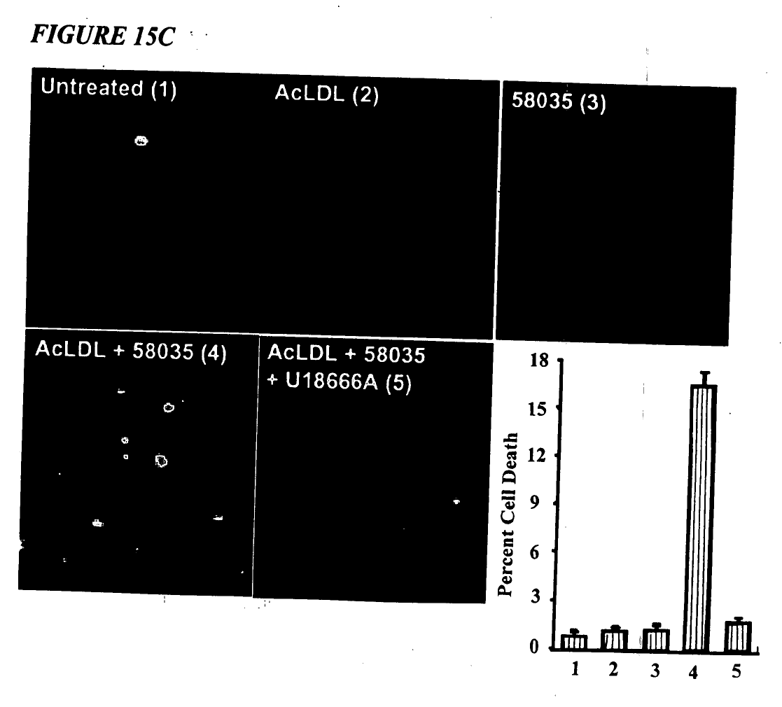

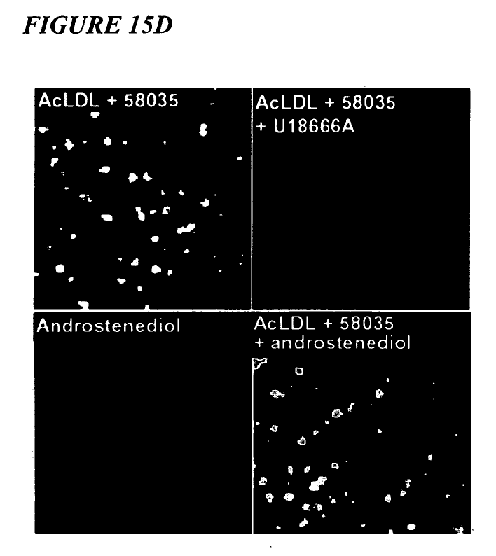

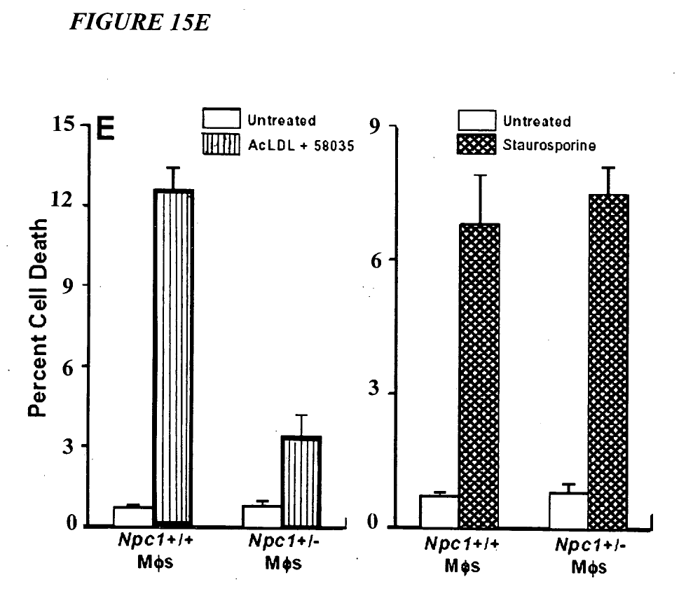

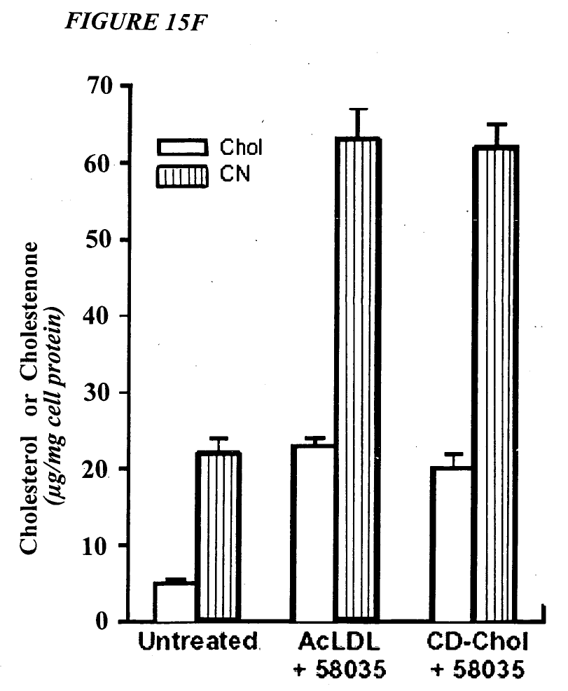

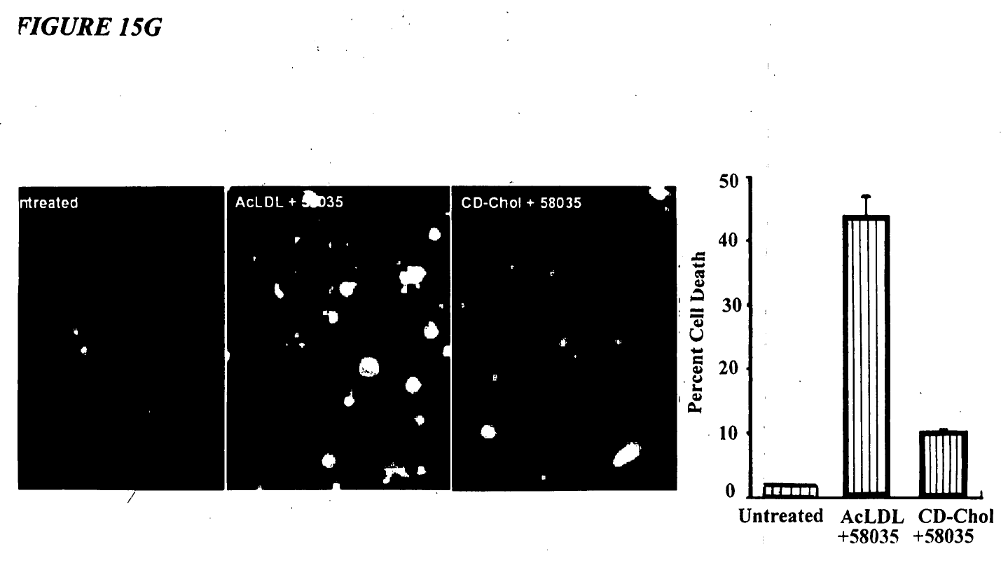

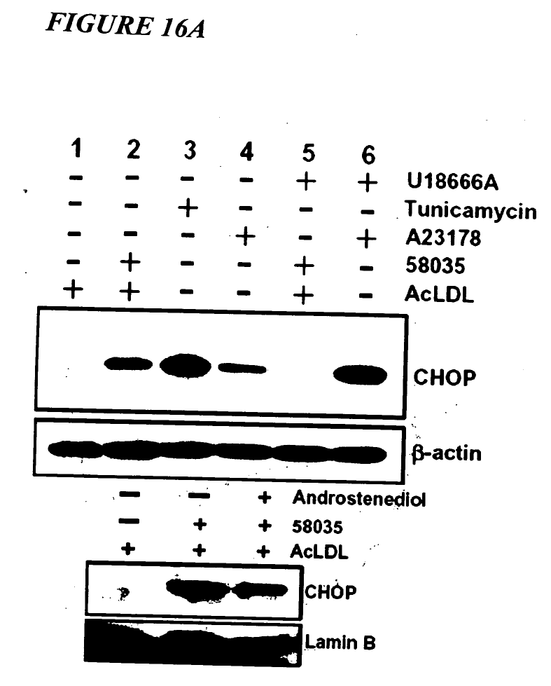

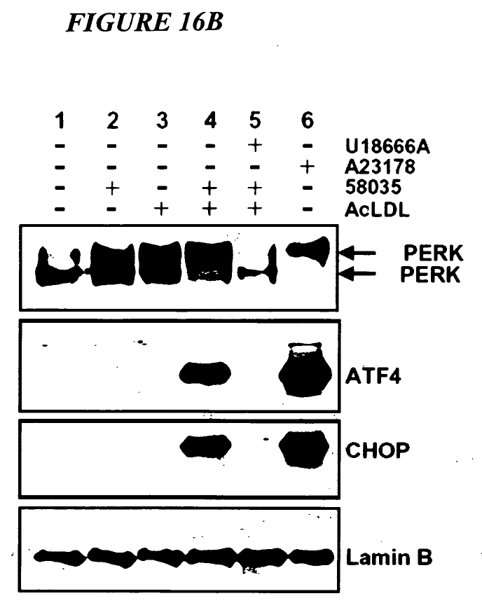

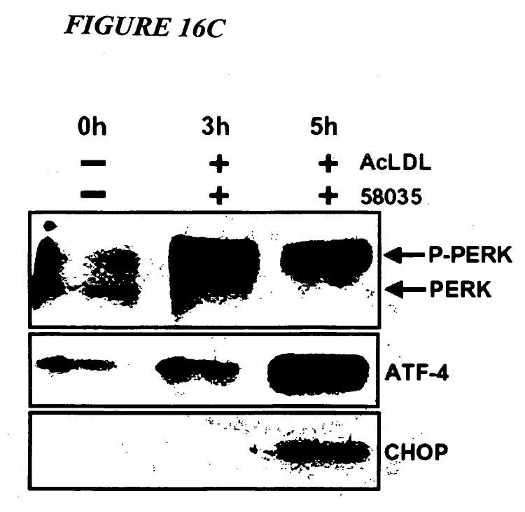

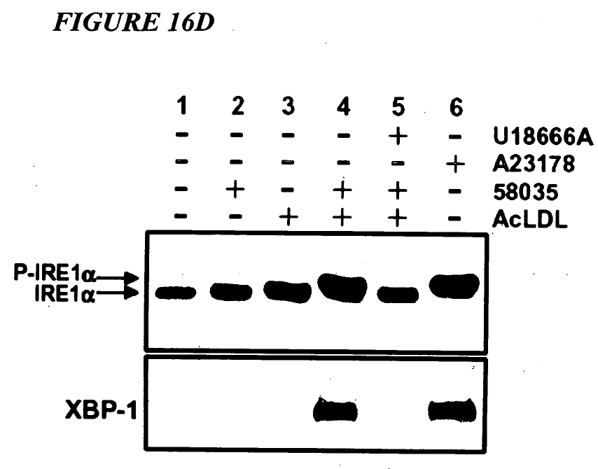

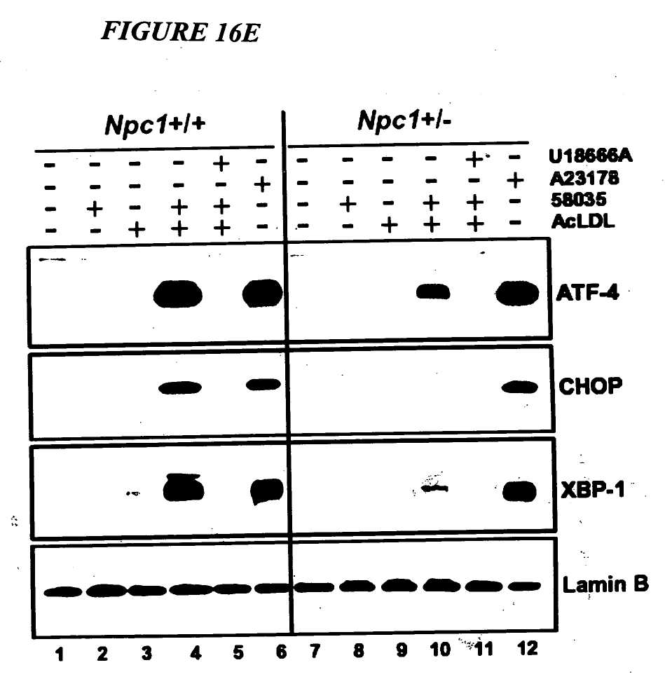

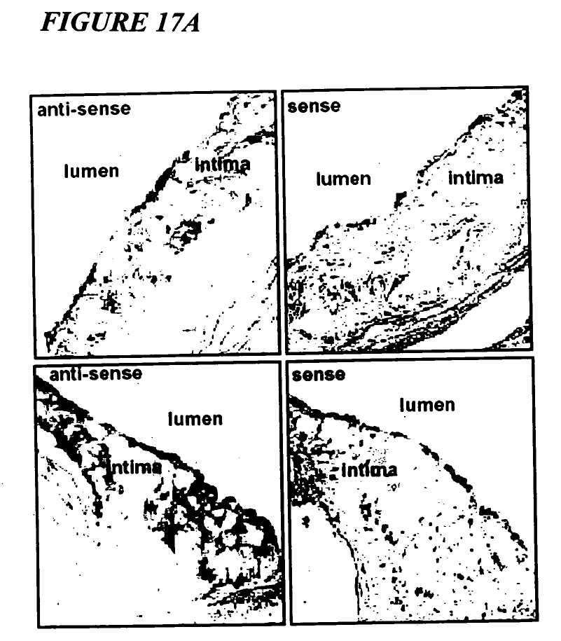

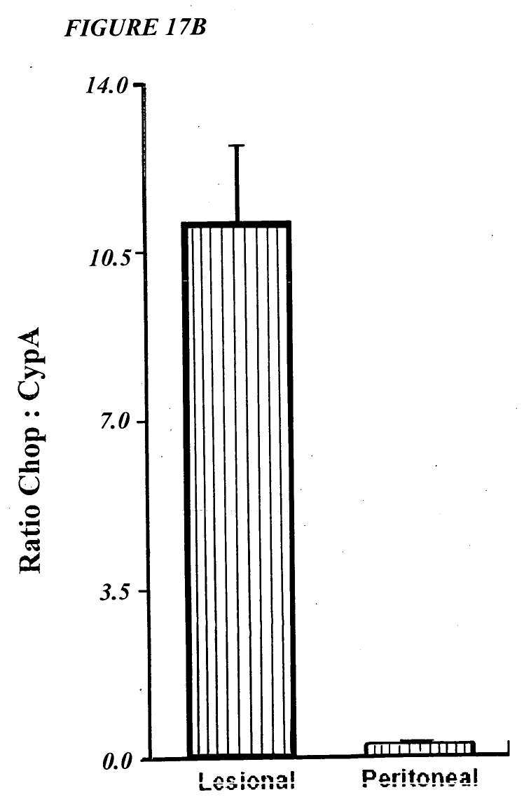

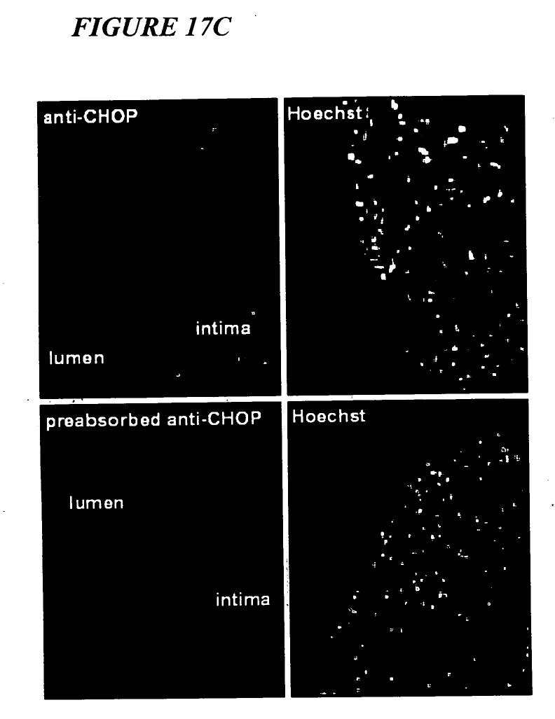

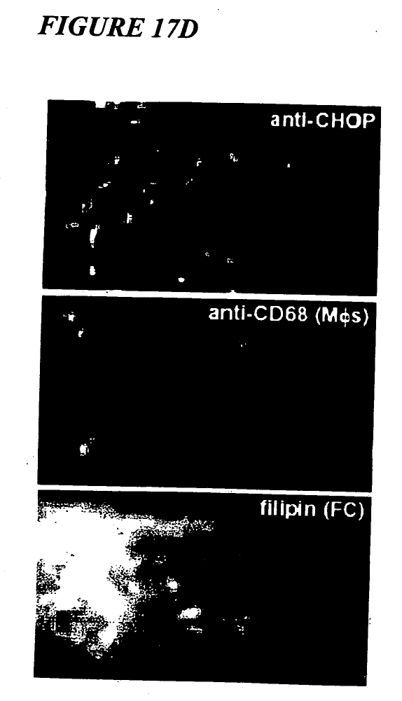

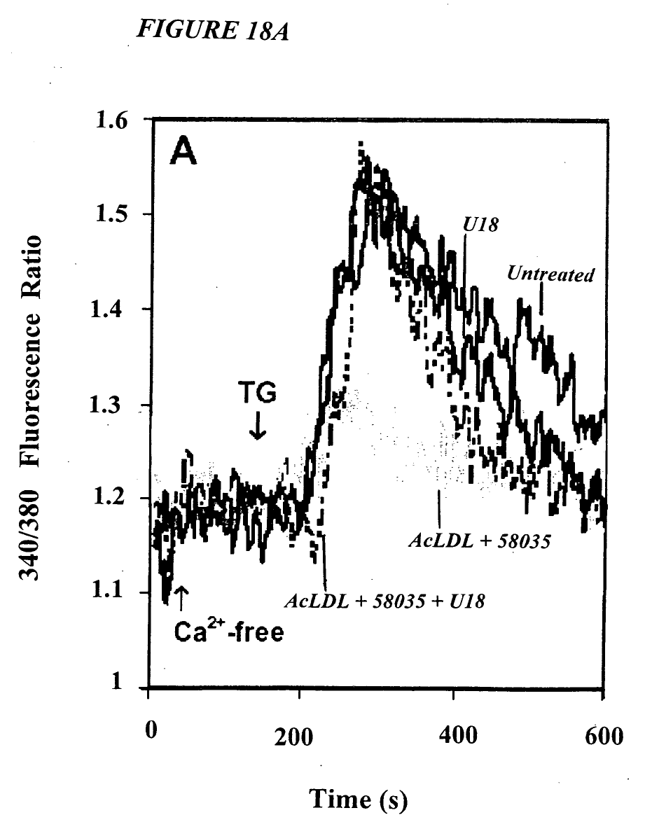

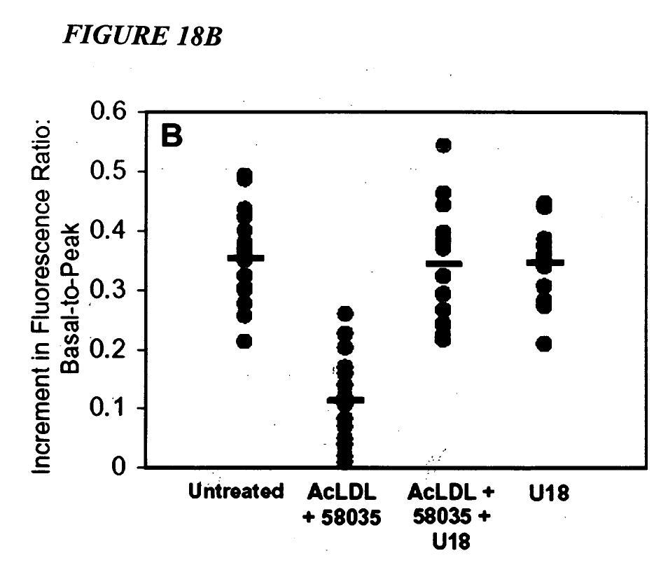

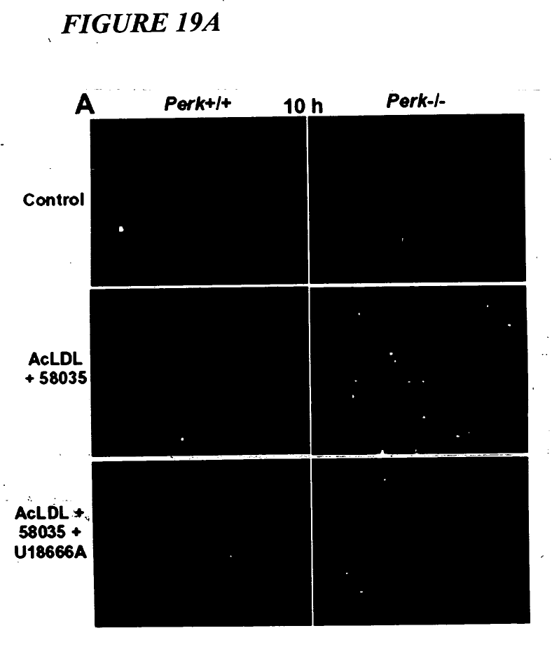

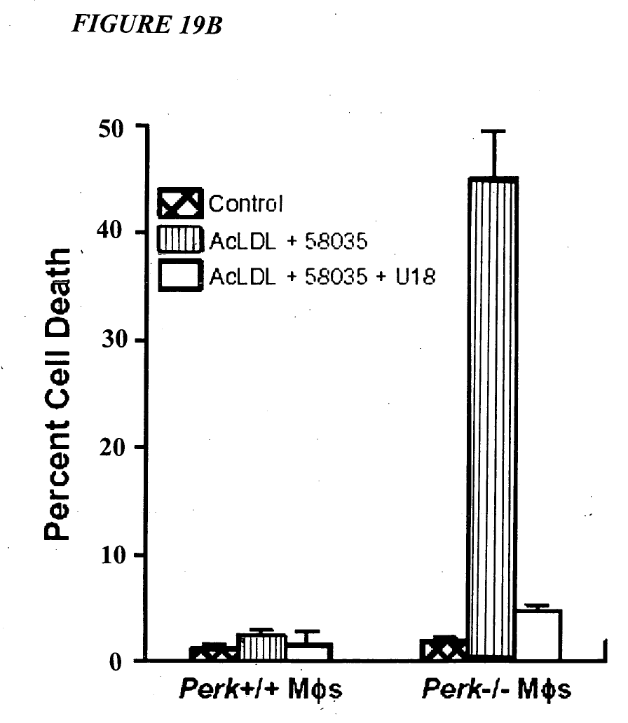

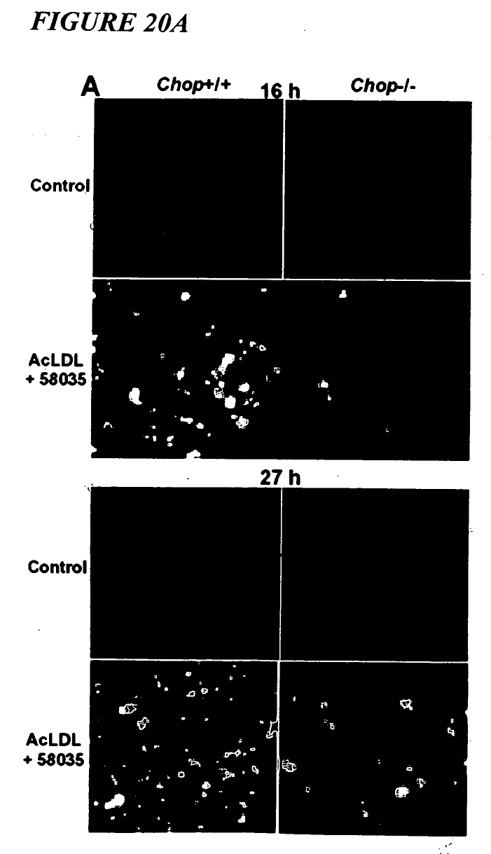

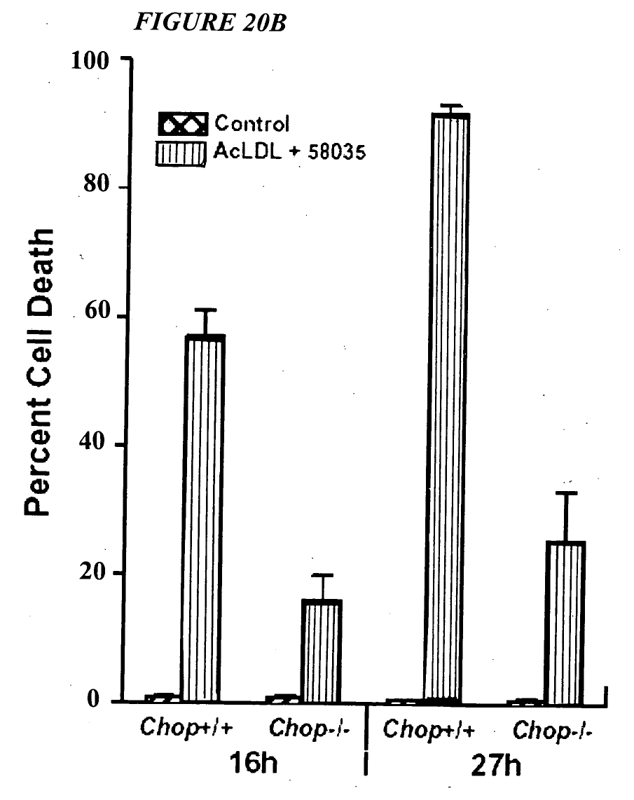

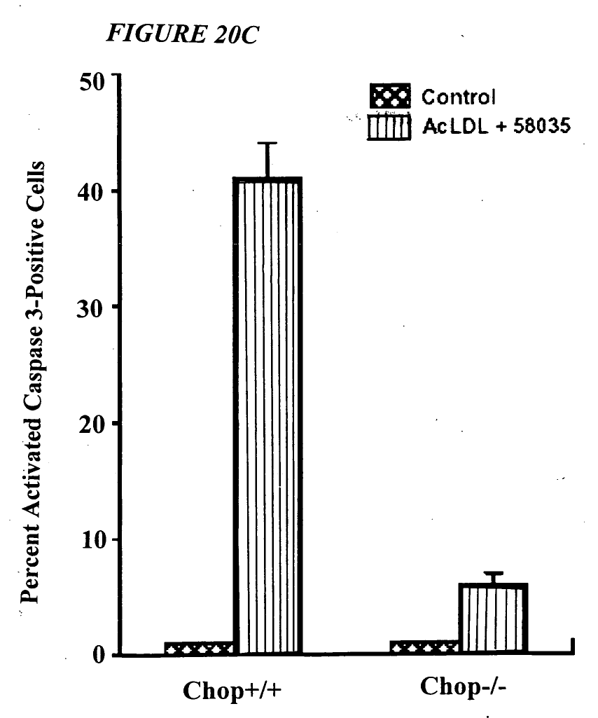

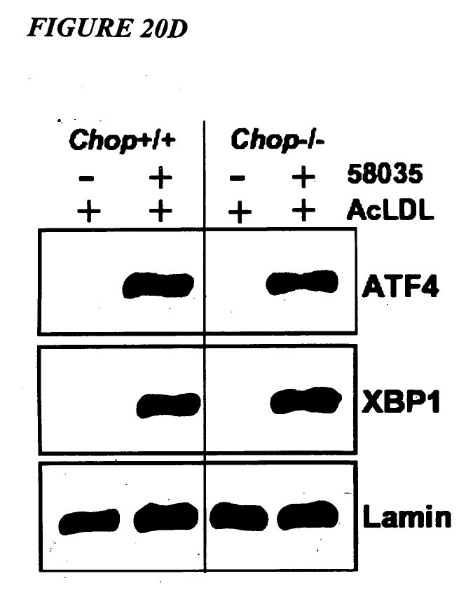

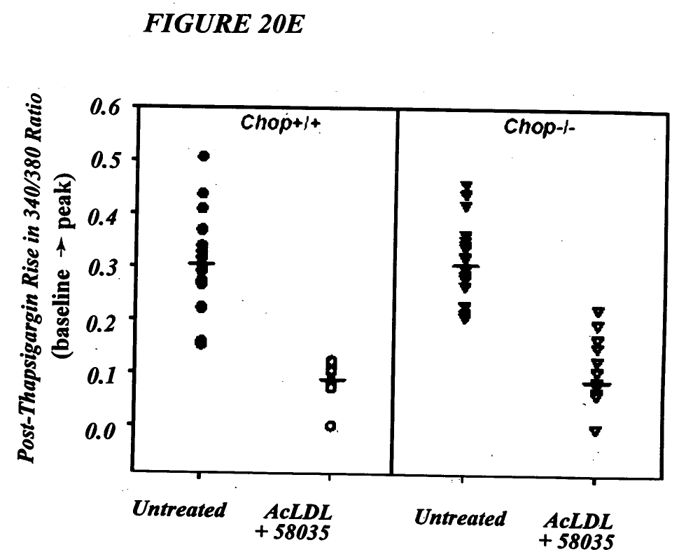

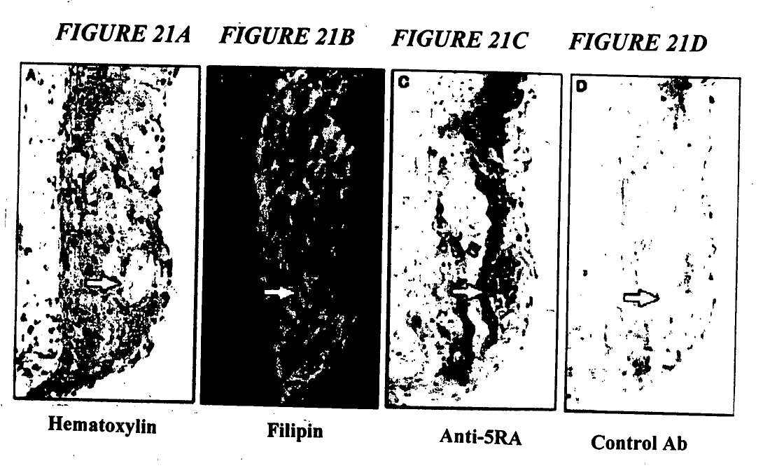

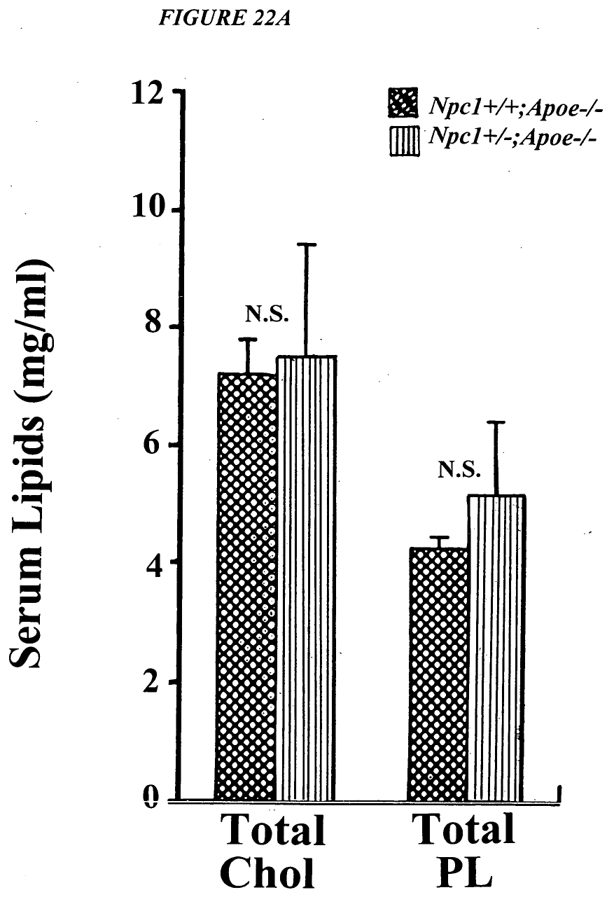

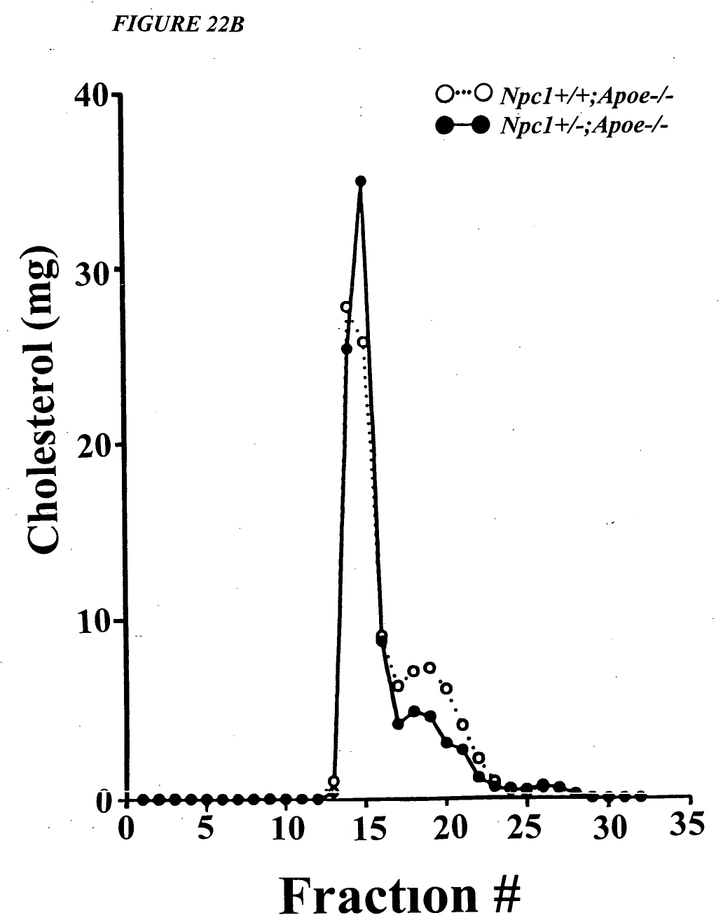

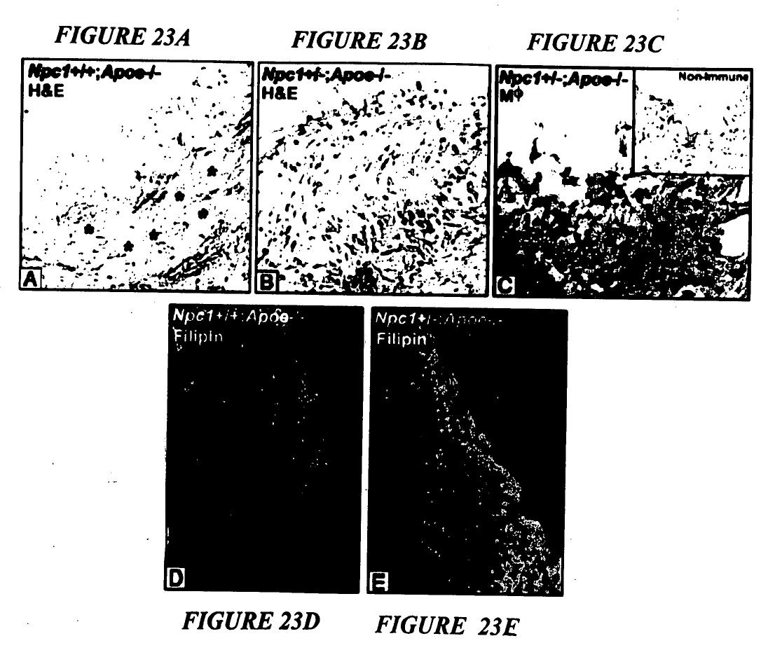

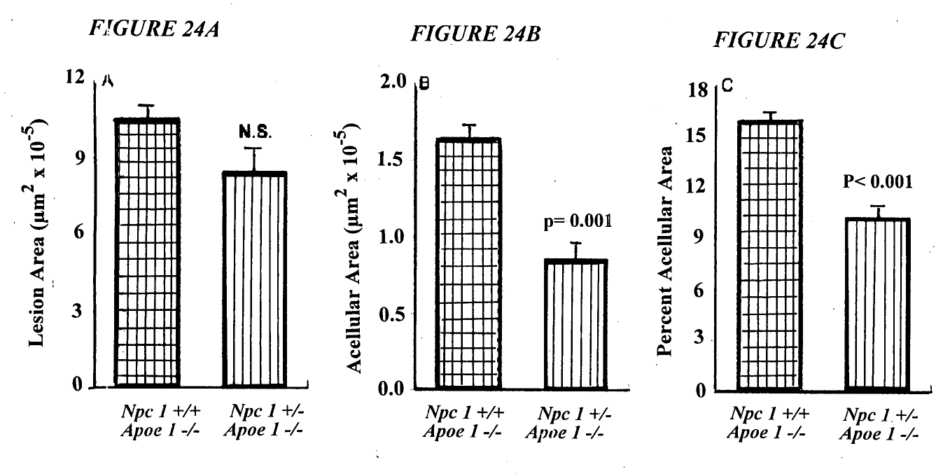

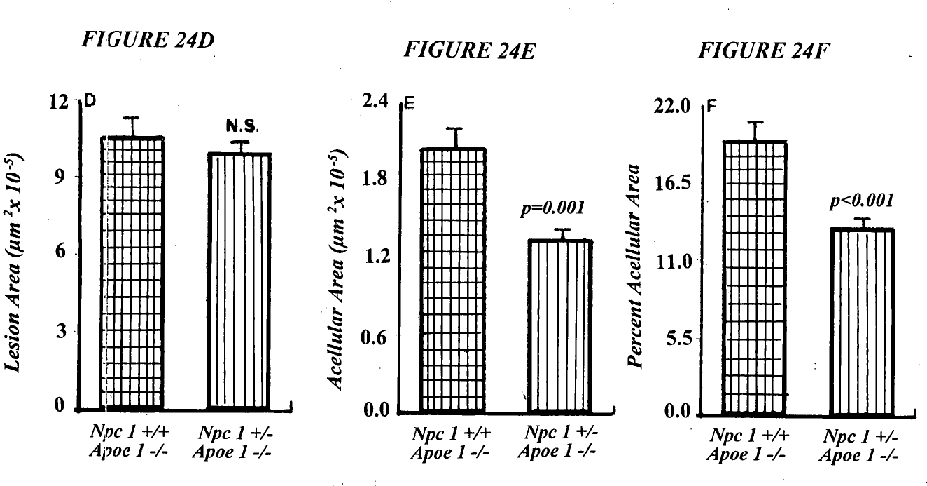

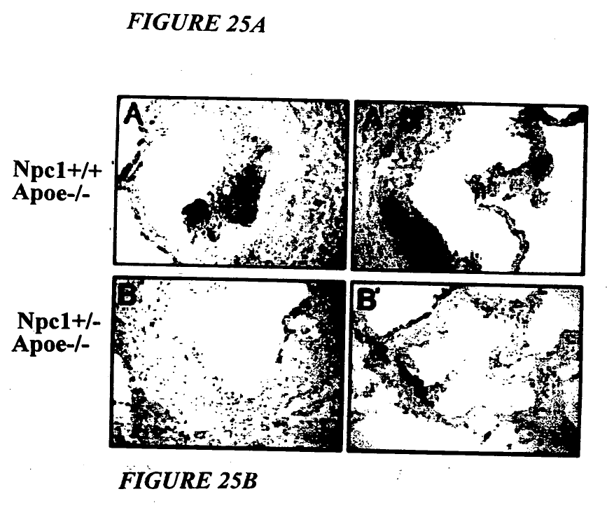

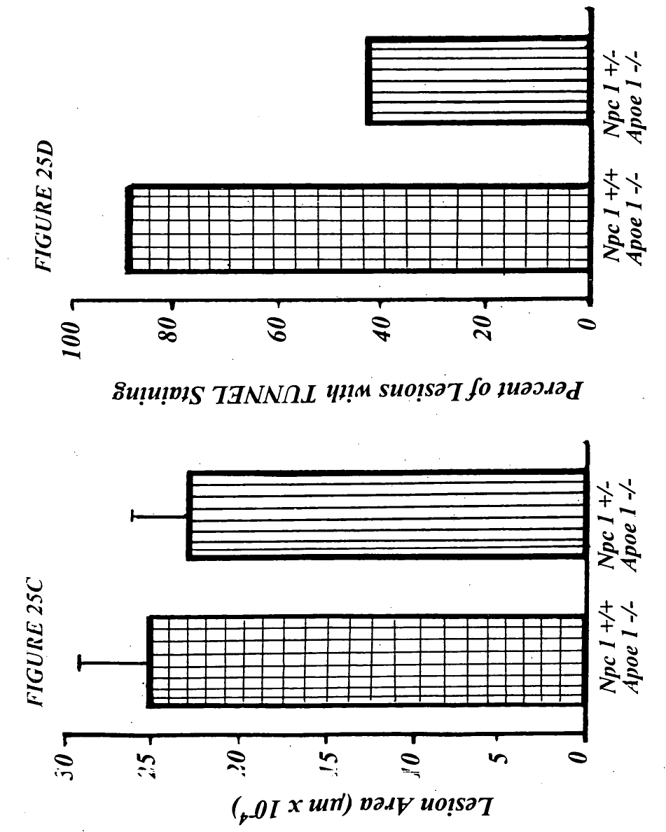

These figures show plasma lipids and lipoprotein profiles of E0 and NPC1/E0 mice. The plasma of 26 E0 mice (14 females and 12 males; cross-hatched bars) and 9 NPC1/E0 mice (3 females and 6 males; solid bars) were assayed for cholesterol and phospholipid concentrations (FIG. 3A), and pooled plasma samples from two male E0 mice (open circles) and two male NPC1/E0 mice (closed circles) were subjected to FPLC gel-filtration fractionation (FIG. 3B). The differences between the two groups of mice for both cholesterol and phospholipid levels in FIG. 3A were not statistically significant. The difference between the two groups of mice in the FPLC peak around fraction #18 in FIG. 3B was not observed in additional experiments.

Legend: cross-hatched bars =E0; solid bars =NPC1/E0; open circles =E0; solid circles =NPC1/E0.

FIGS. 4A, 4B and 4C

These figures show atherosclerotic lesion area and necrotic area in the proximal aorta of E0 and NPC1/E0 mice. Six sections of proximal aorta from 26 E0 mice (14 females and 12 males; cross-hatched bars) and 9 NPC1/E0 mice (3 females and 6 males; solid bars) were assayed for average atherosclerotic lesion area (FIG. 4A) and necrotic area (FIG. 4B); in FIG. 4C, the data are expressed as percent necrotic area ([necrotic area +lesion area]×100).

FIGS. 5A and 5B

These figures show hematoxylin- and Oil Red O-stained sections of lesions from an E0 mouse and an NPC1/E0 mouse.

Adjacent sections of a proximal aortic lesion from a male E0 mouse were stained with hematoxylin (FIG. 5A, left panel) or Oil Red 0 (FIG. 5A, right panel). Similar staining was done for sections from a male NPC1/E0 mouse in FIG. 5B, left panel and right panel. The asterisks in FIG. 5A (left panel) depict acellular areas; these areas stained only weakly for collagen (data not shown). The closed arrowheads in FIG. 5B (left panel) show cellular areas, and the open arrow in FIG. 5B (left panel) shows an area containing cholesterol crystals. Note that the cellular areas stain more intensely with Oil Red 0, which preferentially stains neutral lipids like cholesteryl ester.

FIG. 6A

This figure shows that cholesterol efflux to Apo-A1 is defective in free cholesterol-loaded macrophages. Mouse peritoneal macrophages were incubated for 5 h with 100 μg/ml ³H-cholesterol-labeled acetyl-LDL alone (cholesteryl ester loading) or plus 10 μg/ml of the ACAT inhibitor 58035 (free cholesterol loading). The cells were then incubated with 15 μg/ml apo-A1 for 2.5 h, and efflux of ³H-cholesterol was measured. The data are expressed as the percentage of total cellular ³H-cholesterol.

FIG. 6B

This figure shows that cholesterol efflux to HDL₂ is modestly impaired in free cholesterol-loaded macrophages. Cells were treated as in FIG. 6A except following cholesterol loading the cells were incubated with 20 μg/ml HDL₂ for 2.5 h. Efflux was measured and data are presented as in FIG. 6A.

FIG. 6C

This figure shows that phospholipid efflux to Apo-A1 is defective in free cholesterol-loaded macrophages. Cells were labeled for 24 h with ³H-choline chloride and then treated as in FIG. 6A, except that phospholipid efflux was measured and the data are expressed as percentage of total cellular ³H-phospholipids.

FIG. 6D

This figure shows cells treated and cholesterol efflux measured as in FIG. 6A, except that the time of apoA-I incubation was varied as indicated on the x-axis.

FIG. 6E

This figure shows cells labeled and treated as in FIG. 6C. Aliquots of free cholesterol-loaded cells were incubated for 15 min at 37° C. in the absence or presence of 0.5% or 0.2% methyl-α-cyclodextrin (CD). This treatment removes about 30% of total cellular cholesterol. All cells were then chased with media containing 15 μg/ml apoA-I for 3.33 h and phospholipid efflux was measured as in FIG. 6C.

FIG. 7A

This figure shows that ABCA1 protein is decreased in free cholesterol-loaded macrophages. Mouse peritoneal macrophages were incubated for 5 or 7 h with 100 μg/ml acetyl-LDL in DMEM, 0.2% BSA, in the absence (CE) or presence (FC) of 58035. Aliquots of total cell protein were then subjected to immunoblot analysis for ABCA1 and the standards β-actin or β-integrin.

FIG. 7B

This figure shows that membrane-associated ABCA1 protein is decreased in free cholesterol-loaded macrophages. Cells were treated as in FIG. 7A except that aliquots of cell-surface protein instead of total protein were used for immunoblot analysis of ABCA1 expression.

FIG. 8A

This figure shows that ABCA1 mRNA levels are not substantially altered in free cholesterol-loaded macrophages. Mouse peritoneal macrophages were incubated for 5 h with 100 μg/ml acetyl-LDL in DMEM, 0.2% BSA, in the absence (CE) or presence (FC) of 58035. Total RNA was extracted from the cells, and the ratio of ABCA1:β-actin mRNA was determined by quantitative PCR.

FIG. 8B

This figure shows that free cholesterol-loaded macrophages demonstrate increased degradation of ABCA1 protein. Macrophages were pre-incubated for 14 h with 50 μg/ml acetyl-LDL in DMEM, 0.2% BSA, in the absence (CE) or presence (FC) of 58035. The cells were then incubated for 5 h with 100 μg/ml acetyl-LDL in DMEM, 0.2% BSA, in the absence or presence of 58035, respectively, with no further additions (Control) or in the presence of 10 μg/ml cycloheximide, 50 μM ALLN, or 50 μM lactacystin as indicated. Aliquots of cell lysates were then assayed for ABCA1 and β-actin protein by immunoblot analysis.

FIG. 9A

This figure shows that partial NPC1 deficiency restores ABCA1-mediated cholesterol efflux in FC-loaded macrophages.

Macrophages from wild-type (NPC+/+) and heterozygous (NPC+/−) NPC mice, all on the apoE knockout/C57 background, were incubated with medium containing 100 μg/ml ¹²⁵I-acetyl-LDL for 1, 2, 4, or 6 h, after which cholesterol esterification was assayed. In this experiment, the uptake and degradation of ¹²⁵I-acetyl-LDL and in vitro ACAT activity in the presence of excess cholesterol were similar between the two cell genotypes.

FIG. 9B This figure shows macrophages from wild-type and heterozygous NPC mice, all on the apoE knockout/C57 background, incubated for 5 h with medium containing 100 μg/ml ³H-cholesterol-labeled acetyl-LDL in DMEM, 0.2% BSA, in the presence of 10 μg/ml 58035. The macrophages were then incubated for 18 h in the same medium containing 15 μg/ml of apoA-1 and efflux of ³H-cholesterol was measured as described in FIG. 6.

FIG. 9C

This figure shows the assay performed as in FIG. 9B, except following cholesterol loading, cells were incubated in medium containing 20 μg/ml HDL₂.

FIG. 9D

This figure shows the assay performed as in FIG. 9B, except the 18 h apoA-1 incubation was done in the presence of 200 μM glyburide (GLYB) or 200 μM ortho-vanadate as indicated.

FIG. 10

This figure shows that partial NPC1 deficiency restores ABCA1 protein expression in free cholesterol-loaded macrophages. Macrophages from wild-type and heterozygous NPC mice, all on the apoe knockout/C57 background, were incubated for 5 h with medium containing 100 μg/ml acetyl-LDL in DMEM, 0.2% BSA, in the absence (CE) or presence (FC) of 10 μg/ml 58035. Aliquots of total cell protein (top) or cell-surface protein (bottom) were then subjected to immunoblot analysis for ABCA1 and the standards β-actin or β1-integrin.

FIG. 11A

This figure shows that low dose amphipathic amines restore ABCA1-mediated cholesterol efflux in free cholesterol-loaded macrophages. Peritoneal macrophages from C57 mice were incubated for 5 h with 100 μg/ml ³H-cholesterol-labeled acetyl-LDL in DMEM, 0.2% BSA, in the presence of 10 μg/ml 58035. The macrophages were then incubated for 6 h in the same medium containing 15 μg/ml of apoA-I in the absence or presence of the indicated concentrations of U18666A, and efflux of ³H-cholesterol was measured. The dotted line in each graph indicates the percentage of ³H-cholesterol efflux in the absence of U18666A.

FIG. 11B

This figure shows an assay conducted as in FIG. 11A, except the indicated concentrations of imipramine were used in place of U18666A.

FIG. 12A

This figure shows that 70 nM U18666A restores ABCA1-mediated cholesterol efflux in FC-loaded macrophages and enhances efflux in macrophages incubated long-term with acetyl-LDL. Efflux assay was conducted as described in FIG. 11A except 70 nM U18666A was used, and the apoA-1 incubation time was varied as indicated.

FIG. 12B

This figure shows an efflux assay conducted as in FIG. 12A, except that 20 μg/ml HDL₂ was the cholesterol acceptor.

FIG. 12C

This figure shows macrophage cells incubated with 100 μg/ml acetyl-LDL, without 58035, for 5 h and then incubated for a further 18 h with acetyl-LDL in the absence or presence of 70 nM U18666A.

FIG. 13

This figure shows that 70 nM U18666A restores the level of ABCA1 protein in free cholesterol-loaded macrophages. Macrophages were pre-incubated for 14 h with 50 μg/ml acetyl-LDL in DMEM, 0.2% BSA, in the absence (CE) or presence (FC) of 58035. The cells were then incubated for 5 h with 100 μg/ml acetyl-LDL in DMEM, 0.2% BSA, in the absence or presence of 58035, respectively, with no further additions (Control) or in the presence of 70 nM U18666A. Aliquots of total cell protein (top panel) or cell-surface protein (bottom panel) were then subjected to immunoblot analysis for ABCA1 and the standards β-actin or β1-integrin.

FIG. 14A

This figure shows LDL receptor knockout mice fed a diet containing cholesterol and saturated fat for 12 weeks in the absence or presence of 0.75 mg/kg/d U18666A (10 mice per group). Plasma was assayed for total cholesterol.

FIG. 14B

This figure shows mice treated as in FIG. 14A and plasma assayed for total HDL.

FIG. 14C

This figure shows mice treated as in FIG. 14A and the proximal aorta assayed for total atherosclerotic lesion cross-sectional area. Asteriks denote statistically significant differences between drug and control groups (p<0.05 by the Student's t test).

FIG. 14D

This figure shows mice treated as in FIG. 14A and the proximal aorta assayed for the area of acellular regions. Asteriks denote statistically significant differences between drug and control groups (p<0.05 by the Student's t test).

FIG. 14E

This figure shows mice treated as in FIG. 14A and the proximal aorta assayed for lipid core regions. Asteriks denote statistically significant differences between drug and control groups (p<0.05 by the Student's t test).

FIG. 15A

FIGS. 15A-15G show that FC-induced apoptosis in macrophages is associated with cholesterol trafficking to the ER, not to the plasma membrane. This figure shows the esterification of [3H]cholesterol (a measure of cholesterol trafficking to ER membranes) in cultured mouse peritoneal macrophages incubated for 5 h with medium containing 50 μg/ml [³H]cholesterol-labeled acetyl-LDL in the absence or presence of 70 nM U18666A. Shown is the mean±S.E.M (n=3) of [³H]cholesteryl ester formed (in cpm/mg cell protein).

FIG. 15B

This figure shows the accessibility of cellular cholesterol to cholesterol oxidase (a measure of plasma membrane cholesterol) in fixed macrophages after incubation for 4 h with medium alone or containing 50 μg/ml acetyl-LDL in the absence or presence of 10 μg/ml 58035 or 70 nM U18666A. Shown is the mass (in μg/mg cell protein) of cholesterol (Chol, open bars), which is the cholesterol oxidase-inaccessible pool of intracellular cholesterol, and of cholestenone (CN, striped bars), which is the cholesterol oxidase-accessible pool of plasma membrane cholesterol.

FIG. 15C

This figure shows Alexa-488-annexin V staining (white spot) and propidium iodide staining (white spot) to assess death of macrophages incubated for 8 h under the same conditions as in FIG. 15A, with the inclusion of two additional controls, acetyl-LDL alone and 58035 alone. Representative fluorescence images and quantitative cell death data from five fields of cells for each condition are shown, expressed as the percent of total cells that stained with annexin V or propidium iodide (mean±S.E.M.; n=5 fields of cells, where each field had ˜200 cells). The numbers under each bar in the graph shown refer to the 5 conditions depicted by the 5 images shown.

FIG. 15D

This figure shows Alexa-488-annexin V and propidium iodide staining of macrophages incubated for 16 h with medium containing 100 μg/ml acetyl-LDL+10 μg/ml 58035; acetyl-LDL+58035 +70 nM U18666A; 3.3 μM androstenediol alone; or acetyl-LDL+58035 +3.3 μM androstenediol.

FIG. 15E

This figure shows quantification of annexin V and propidium iodide staining of macrophages from wildtype (Npc1+/+) or Npc1+/−mice incubated for 8.5 h with medium alone, medium containing 50 μg/ml acetyl-LDL plus 10 μg/ml 58035, or medium containing 50 nM staurosporine. Shown are the percent of total cells that stained with annexin V or propidium iodide (mean±S.E.M.; n=5 fields of cells, where each field had ˜200 cells).

FIG. 15F

This figure shows alteration in the mass of the cholesterol oxidase-accessible pool of plasma membrane cholesterol by incubation of macrophages with medium alone, medium containing 25 μg/ml acetyl-LDL plus 10 μM 58035, or medium containing 5 mM methyl-β-cyclodextrin:cholesterol (5:1 mass ratio) plus 10 μM 58035 (CD-Chol) for 4 h. The data are displayed as in FIG. 15B.

FIG. 15G

This figure shows Alexa-488-annexin V and propidium iodide staining of macrophages incubated for 8.5 h in the absence or presence of CD-cholesterol, using the medium described in FIG. 15E. Representative fluorescence images and quantitative data are shown (mean±S.E.M.; n=5 fields of cells, where each field had ˜200 cells).

FIG. 16A

FIGS. 16A-16E show that FC loading of macrophages activates the UPR. In the top panel of this figure it shows CHOP and β-actin immunoblots of whole-cell extracts of macrophages incubated under control or FC-loading conditions. The macrophages were incubated for 5 h in medium containing the following additions: 100 μg/ml acetyl-LDL (CE loading, lane 1); 100 μg/ml acetyl-LDL plus 10 μg/ml 58035 (FC loading, lane 2); 2.5 μg/ml tunicamycin or 2 μg/ml A23187 (lanes 3 and 4, positive controls); acetyl-LDL and 58035 in the presence of 70 nM U18666A (inhibition of cholesterol trafficking to the ER, lane 5); and A23187 plus U18666A (control of U18666A effect, lane 6). In the bottom panel, macrophages were incubated with acetyl-LDL alone, acetyl-LDL+58035, or acetyl-LDL+58035 +3.3 μM androstenediol and then subjected to immunoblot analysis for CHOP and, as a loading control, lamin B.

FIG. 16B

This figure shows PERK, ATF-4, CHOP, and lamin B immunoblots of nuclei-free or nuclear extracts of macrophages incubated under control or FC-loading conditions. Macrophages were incubated for 5 h in medium containing the following additions: no additions (lane 1); 58035 (lane 2); 100 μg/ml acetyl-LDL (CE loading, lane 3); 100 μg/ml acetyl-LDL plus 10 μg/ml 58035 (FC loading, lane 4); acetyl-LDL and 58035 in the presence of 70 nM U18666A (inhibition of cholesterol trafficking to the ER, lane 5); and 2 μg/ml A23187 (positive control, lane 6). For detection of PERK, nuclei-free cell extracts were immunoprecipitated with anti-PERK antiserum and then immunoblotted, while for detection of ATF-4 and CHOP, immunoblots were performed on nuclear extracts. Lamin B, a nuclear protein, was used as the loading control.

FIG. 16C

This figure shows PERK, ATF-4, CHOP, and lamin B immunoblots of nuclei-free or nuclear extracts of macrophages incubated under control or FC-loading conditions. Macrophages were incubated for 3 or 5 h with medium containing 100 μg/ml acetyl-LDL plus 10 μg/ml 58035 (FC loading), or extracted prior to the incubation period (0 h), and then immunoblotted for PERK, ATF-4, and CHOP immunoblots as in FIG. 16B.

FIG. 16D

This figure shows IRE1α and XBP-1 immunoblots of nuclei-free or nuclear extracts, respectively, of macrophages incubated under the same control or FC-loading conditions as in FIG. 16B.

FIG. 16E

This figure shows ATF-4, CHOP, XBP-1, and lamin B immunoblots of macrophages from Npc1+/+ or Npc1+/−mice that were incubated under the same conditions as in FIG. 16B and FIG. 16D.

FIG. 17A

FIGS. 17A-17D show that CHOP is expressed in the atherosclerotic lesions of Apoe−/− mice. FIG. 17A shows Chop in-situ histohybridization of sections of proximal aortic atherosclerotic lesions from Apoe−/− mice fed the Western-type diet for 13 weeks. Representative images using the anti-sense Chop probe are shown in the two left panels, while adjacent sections stained with the control, sense probe are shown in the two right panels. The sections were counter-stained with Fast Red to show the nuclei of the lesional cells.

FIG. 17B

This figure shows quantitative Chop RT-PCR of RNA from lesional macrophages, using laser capture microdissection, and from resident peritoneal macrophages using mice similar to those in FIG. 17A. Shown is the mean±S.E.M (n=3 Taqman repeats of the RNA samples) of the Chop:CypA (standard) ratio.

FIG. 17C

This figure shows anti-CHOP and Hoechst-33258 (nuclear) double immunofluorescence microscopy of sections of a proximal aortic lesion from a mouse similar to that in FIG. 17A. Shown in the top left and right panels is a representative section showing the CHOP and nuclear signals, respectively. Shown in the bottom left panel is the CHOP signal after absorption of the antibody with its cognate peptide; the bottom right panel shows the nuclear staining of this section.

FIG. 17D

This figure shows anti-CHOP, anti-CD68 (macrophages), and filipin (FC) staining of three sections of a proximal aortic lesion from a mouse similar to that in FIG. 17A.

FIG. 18A

FIGS. 18A and 18B show that FC loading of macrophages leads to early depletion of ER calcium stores. FIG. 18A shows the assessment of ER calcium stores in macrophages incubated for 2.5 h with medium alone (Untreated) or containing 70 nM U18666A, 100 μg/ml acetyl-LDL+10 μg/ml 58035, or acetyl-LDL+58035 +70 nM U18666A. Shown are representative tracings of the 340/380-nm Fura-2 fluorescence ratio in an individual macrophage from each treatment group before and after addition of 1 μM thapsigargin.

FIG. 18B

This figure shows a plot of basal-to-peak Fura-2 fluorescence ratio after addition of thapsigargin in individual cells in each treatment group, with the mean increment denoted by the line.

FIGS. 19A and 19B

These figures show that disruption of the upstream PERK branch of the UPR enhances FC-induced apoptosis in macrophages. Alexa-488-annexin V and propidium iodide staining of macrophages from wildtype (Perk+/+) or Perk−/− mice incubated for 10 h with medium containing 100 μg/ml acetyl-LDL alone; 100 μg/ml acetyl-LDL+10 μg/ml 58035; or acetyl-LDL+58035 +70 nM U18666A. Representative fluorescence images are shown in FIG. 19A, and quantitative apoptosis data from five fields of cells for each condition are shown in FIG. 19B, expressed as the percent of total cells that stained with annexin V or propidium iodide (mean±S.E.M.; n=5 fields of cells, where each field had ˜200 cells).

FIGS. 20A and 20B

FIGS. 20A-20E show that disruption of CHOP attenuates FC-induced apoptosis in macrophages. FIGS. 20A and 20B show Alexa-488-annexin V and propidium iodide staining of macrophages from wildtype (Chop+/+) or Chop−/− mice incubated for 16 h or 27 h with medium containing 100 μg/ml acetyl-LDL alone or acetyl-LDL+58035. Representative fluorescence images are shown in FIG. 20C, and quantitative cell death data from five fields of cells for each condition are shown in FIG. 20D, expressed as the percent of total cells that stained with annexin V or propidium iodide (mean±S.E.M.; n=5 fields of cells, where each field had ˜200 cells).

FIG. 20C

This figure shows percent of control and FC-loaded Chop+/+ and Chop−/− macrophages with activated daspase-3.

FIG. 20D

This figure shows ATF-4, XBP-1, and lamin B immunoblots of macrophages from Chop+/+ or Chop−/− mice that were incubated under the same conditions as in FIGS. 16B and 16D.

FIG. 20E

This figure shows a plot of basal-to-peak Fura-2 fluorescence ratio after addition of thapsigargin, a measure of ER calcium stores, in control and FC-loaded macrophages from Chop+/+ or Chop−/− mice, with the mean increment denoted by the line. The experimental conditions were the same as those described for FIGS. 18A and 18B.

FIGS. 21A-21D

These figures show the characterization of acellular areas in the advanced atherosclerotic lesions of Apoe−/− mice. Adjacent sections of proximal aortic lesions from 25-week-old cholesterol-fed Apoe−/− mice were stained with hematoxylin (FIG. 21A), filipin (FIG. 21B), anti-type A scavenger receptor antibody (FIG. 21C), or control antibody (FIG. 21D). Arrows indicate one of several acellular areas in each figure. (X150.) FIGS. 22A and 22B These figures show the plasma lipids and lipoprotein profile of Npc1+/+; Apoe−/− and Npc1+/−; Apoe−/− mice. FIG. 22A: plasma samples of 25-week-old cholesterol-fed Npc1+/+; Apoe−/− mice (14 females and 12 males, cross-hatched bars) and Npc1+/−; Apoe−/− mice (3 females and 6 males, solid bars) were assayed for cholesterol (Chol) and phospholipid (PL) concentrations. The differences in both cholesterol and phospholipid levels between the two groups of mice were not statistically significant. FIG. 22B: pooled plasma samples from two male Npc1+/+; Apoe−/− (◯) and two male Npc1+/−; Apoe−/− (●) mice were subjected to fast performance liquid chromatography gel-filtration fractionation. The small difference between the two groups of mice in the fast performance liquid chromatography peak around fraction 18 was not observed in repeat experiments.

FIGS. 23A-23E

These figures show hematoxylin, macrophage, and filipin staining of proximal aortic lesion sections from male Npc1+/+; Apoe−/− and Npc1+/−; Apoe−/− mice. FIGS. 23A and 23B: sections of proximal aortic lesions from 25-week-old cholesterol-fed male Npc1+/+; Apoe−/− (FIG. 23A) and Npc1+/−; Apoe−/− mice (FIG. 23B). Sections were stained with hematoxylin and eosin (H&E). (X225.) Asterisks indicate acellular areas. The lesion from the Npc1+/−; Apoe−/− mouse (FIG. 23B) is markedly more cellular than the lesion from the Npc1+/+; Apoe−/− mouse (FIG. 23A). FIG. 23C: these cells were identified as macrophages (Mφ) by immunohistochemistry. Inset shows a nonimmune control. FIGS. 23D and 23E: lesions from these mice were stained with filipin. Overall lesional accumulation of FC was not markedly different in the two lesions.

FIGS. 24A-24F These figures show quantification of atherosclerotic lesion area and acellular area in the proximal aortae of Npc1+/+; Apoe−/− and Npc1+/−; Apoe−/− mice. Analyses were conducted =1 year apart on two separate groups of mice. In experiment 1 (FIGS. 24A-24C), the proximal aortae from 25-week-old cholesterol-fed Npc1+/+; Apoe−/− mice (14 females and 12 males, cross-hatched bars) and Npc1+/−; Apoe−/− mice (3 females and 6 males, solid bars) were assayed for average atherosclerotic lesion area (FIG. 24A) and acellular area (FIG. 24B). FIG. 24C: the data are expressed as percent acellular area [(acellular area/lesion area)×100]. In experiment 2 (FIGS. 24D-24F), the same analyses are displayed in FIGS. 24D, 24E, and 24F, respectively. There were 13 Npc1+/+; Apoe−/− mice (5 females and 8 males) and 15 Npc1+/−; Apoe−/− mice (9 females and 6 males). Using the Mann-Whitney U test, we found that the difference in lesion area between the two groups of mice in both experiments was not statistically significant, whereas the difference was highly statistically significant for acellular area (P=0.001) and percent acellular area (P<0.001) in both experiments.

FIGS. 25A-25D

These figures show TUNEL staining of lesions in the proximal aortae of Npc1+/+; Apoe−/− and Npc1+/−; Apoe−/− mice. Atherosclerotic lesions in the proximal aortae of nine 18-week-old cholesterol-fed Npc1+/+; Apoe−/− mice and seven Npc1+/−; Apoe−/− mice were assayed for TUNEL positivity. Examples of representative sections from two Npc1+/+; Apoe−/− mice (FIG. 25A) and two Npc1+/−; Apoe−/− mice (FIG. 25B) are shown. (X20.) Quantification of total lesion area and the percentage of mice that had TUNEL-positive lesions are shown in FIGS. 25C and 25D, respectively. For the TUNEL analysis, two aortic sections were examined per mouse. Using the Mann-Whitney U test, we found that the difference in lesion area between the two groups of mice was not statistically significant, whereas the difference in TUNEL positivity was found to be statistically significant by the X² test (P<0.05).

### DETAILED DESCRIPTION OF THE INVENTION

The following abbreviations are used herein: ACAT: acyl-CoA:cholesterol acyltransferase; E0: apolipoprotein E knockout animal; FC: free cholesterol; LDL: low-density lipoprotein; NPC: Niemann-Pick; NPC1: heterozygous NPC knockout animal; PI: propidium iodide; VLDL: very low-density lipoprotein.

Definitions “ABCA1 ” is used herein to mean “ATP-binding cassette transporter A1”, and is also referred to in the art as “ABC1”.

As used herein, “ACAT” shall mean “acyl-CoA-cholesterol acyltransferase,” which is the enzyme that catalyzes the first committed step in cholesterol ester biosynthesis. Inhibitors of this enzyme are known in the art, and are exemplified by Matsuda (1994) (10).

“Administering” may be effected or performed using any of the methods known to one skilled in the art. The methods comprise, for example, intralesional, intramuscular, subcutaneous, intravenous, intraperitoneal, liposome-mediated, transmucosal, intestinal, topical, nasal, oral, anal, ocular or otic means of delivery.

As used herein, “amphiphilic compounds” include, without limitation, compounds which inhibit cholesterol esterification (e.g., steroids such as progesterone), hydrophobic amines, phenothiazines, ionophores, cytochalasins, lysophosphatides such as lysophosphatidylcholine, lysophosphatidylserine and lysophosphatidylethanolamine, colchicine, nigericin, chloroquine, chlorpromazine, trifluoperazine, monensin and amphipathic amines such as imipramine and U18666A (Lange et al. (1994) J. Biol. Chem. 269(47): 29371-29374).

“Atherosclerotic lesional complications” include, without limitation, necrosis and thinning of the protective fibrous cap. An important aspect of lesion progression is the progression of early, benign lesions (i.e. those not likely to cause clinical disease) into lesions characterized by lesional complications. These complicated lesions are known as vulnerable plaques, because they are at risk of eroding or rupturing, leading to acute vascular thrombosis.

“ApoA-I” shall mean “apolipoprotein A-I”, which is the major protein of high density lipoprotein (HDL).

“Cardiovascular disease” shall include, without limitation, atherosclerotic vascular disease, advanced atherosclerotic lesions, atherosclerosis-associated acute thrombosis, vascular occlusion, tissue infarction, myocardial infarction, aneurism, angina, peripheral vascular disease, stroke, acute occlusive thrombosis or other clinical event associated with atherosclerosis, or other peripheral vascular disease.

As used herein, “cholesterol” includes, without limitation, esterified cholesterol (e.g., cholesteryl esters), and non-esterified cholesterol (e.g., free-cholesterol).

As used herein, “cholesterol-containing particle” includes, without limitation, both naturally occurring and recombinant low density lipoproteins, as well as synthetic cholesterol-containing particles. Cholesterol-containing particles must be able to enter a cell and thereby serve as a vehicle for the importation of cholesterol into the cell.

As used herein, “cholesterol efflux” shall mean the movement of cholesterol from a cell to the cell's exterior, and/or any biochemical step constituting part of such movement. In one embodiment, cholesterol is moved from a cell to a cholesterol acceptor which then transports the cholesterol out of the cell.

As used herein, a “cholesterol-loaded” cell shall mean a cell having a level of cholesterol higher than normal for that cell type. For example, if a human macrophage has a cholesterol level of X, and a human macrophage in question has a cholesterol level of 2×, the human macrophage in question is considered “cholesterol-loaded.” A higher than normal cholesterol level can be any level higher than normal including, for example, 1%, 2%, 5%, 10%, 20%, 50%, and 100% higher than normal. In one embodiment, free cholesterol-loaded cells are formed in culture by human intervention. This is accomplished, for example, by contacting the cells in culture with a cholesterol-containing particle, such as an acetylated low density lipoprotein, under conditions where ACAT is inhibited. If ACAT is not inhibited, then the cells become loaded primarily with cholesteryl esters instead of free cholesterol.

As used herein, the term “composition”, as in pharmaceutical composition, is intended to encompass a product comprising the active ingredient(s) and the inert ingredient(s) that make up the carrier, as well as any product which results, directly or indirectly from combination, complexation, or aggregation of any two or more of the ingredients, or from dissociation of one or more of the ingredients, or from other types of reactions or interactions of one or more of the ingredients.

As used herein, “effective amount” refers to an amount which is capable of treating or preventing a plaque rupture or superficial erosion or treating or preventing or delaying the onset of a disease or disorder or other clinical event described herein, or preventing or delaying the onset of macrophage death. Accordingly, the effective amount will vary with the subject being treated, as well as the condition to be treated. A person of ordinary skill in the art can perform routine titration experiments to determine such sufficient amount. The effective amount of a compound will vary depending on the subject and upon the particular route of administration used. Based upon the compound, the amount can be delivered continuously, such as by continuous pump, or at periodic intervals (for example, on one or more separate occasions). Desired time intervals of multiple amounts of a particular compound can be determined without undue experimentation by one skilled in the art.

As used herein, “HDL” shall mean “high-density lipoprotein.” HDL is the main extracellular acceptor of cholesterol, and transports cholesterol to the liver for excretion.

As used herein, a subject at “increased risk” for cardiovascular disease shall mean any subject possessing known risk factors for such disease, including for example, high LDL, low HDL, diabetes, smoking history, hypertension, obesity, metabolic syndrome, hypercoagulation state, thrombosis history, family history of cardiovascular disease, high lipoprotein(a), high homocysteine, high apolipoprotein B, high CRP, high lipoprotein-associated phospholipase A2, or high myeloperoxidase.

“Inhibiting” the onset of a disorder shall mean either lessening the likelihood of the disorder's onset, or preventing the onset of the disorder entirely. In the preferred embodiment, inhibiting the onset of a disorder means preventing its onset entirely.

“Niemann-Pick C molecule”, abbreviated herein as “NPC”, includes, without limitation, type I and type II molecules. These NPC molecules play an important role in intracellular cholesterol trafficking, particularly in the exit of cholesterol from late endosomes or lysosomes.

As used herein, “pharmaceutically acceptable carrier” means that the carrier is compatible with the other ingredients of the formulation and is not deleterious to the recipient thereof, and encompasses any of the standard pharmaceutically accepted carriers. Such carriers include, for example, 0.01-0.1 M and preferably 0.05 M phosphate buffer or 0.8% saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like.

As used herein, “preventing or delaying a plaque rupture or superficial erosion, or simultaneous or subsequent vascular event” includes ameliorating, suppressing, halting, slowing the progression of, or controlling the plaque rupture or superficial erosion, or simultaneous or subsequent vascular event.

“Subject” shall mean any organism including, without limitation, a mammal such as a mouse, a rat, a dog, a guinea pig, a ferret, a rabbit and a primate. In the preferred embodiment, the subject is a human being.

“Treating” means either slowing, stopping or reversing the progression of a disorder. As used herein, “treating” also means the amelioration of symptoms associated with the disorder.

As used herein, “U18666A” shall mean the amphipathic amine known as 2β-(2-diethlaminoethoxy)-androstenone, 3-β-[2-(diethylamino)ethoxy]androst-5-en-17-one or 3-β[2-(diethylaminoethoxy]androst-5-en-17-one hydrochloride.

Embodiments of the Invention

This invention provides a method for inhibiting macrophage death in a subject having, or at increased risk for developing, cardiovascular disease which comprises administering to the subject an effective amount of an amphiphilic compound or a pharmaceutically acceptable salt thereof which inhibits the intracellular transport of cholesterol within cells, wherein the transport is from an intracellular cholesterol storage site to the endoplasmic reticulum, so as to thereby inhibit macrophage death in the subject.

This invention also provides a method for inhibiting atherosclerotic lesional complications in a subject having, or at increased risk for developing, cardiovascular disease which comprises administering to the subject an effective amount of an amphiphilic compound or a pharmaceutically acceptable salt thereof which inhibits the intracellular transport of cholesterol within cells, wherein the transport is from an intracellular cholesterol storage site to the endoplasmic reticulum, so as to thereby inhibit atherosclerotic lesional complications in the subject.

This invention also provides a method for inhibiting macrophage death in a subject having, or at increased risk for developing, cardiovascular disease which comprises administering to the subject an effective amount of an amphiphilic compound or a pharmaceutically acceptable salt thereof which inhibits free cholesterol-induced death of cells in the subject by inhibiting intracellular transport of cholesterol within the cells, wherein the transport is from an intracellular cholesterol storage site to the endoplasmic reticulum, so as to thereby inhibit macrophage death in the subject.

This invention further provides a method for inhibiting atherosclerotic lesional complications in a subject having, or at increased risk for developing, cardiovascular disease which comprises administering to the subject an effective amount of an amphiphilic compound or a pharmaceutically acceptable salt thereof which inhibits free cholesterol-induced death of cells in the subject by inhibiting intracellular transport of cholesterol within the cells, wherein the transport is from an intracellular cholesterol storage site to the endoplasmic reticulum, so as to thereby inhibit atherosclerotic lesional complications in the subject.

In one embodiment of the above methods the compound inhibits the function of Neiman Pick Cl (NPC1) protein within the cells. In another embodiment of the above methods the compound inhibits expression of Neiman Pick Cl (NPC1) protein within the cells.

This invention further provides a method for inhibiting necrosis, plaque rupture and/or superficial erosion in a subject having, or at increased risk for developing, cardiovascular disease which comprises administering to the subject an effective amount of an amphiphilic compound or a pharmaceutically acceptable salt thereof which inhibits intracellular transport of cholesterol within cells, wherein the transport is from an intracellular cholesterol storage site to the endoplasmic reticulum, so as to thereby inhibit necrosis, plaque rupture and/or superficial erosion in the subject.

In one embodiment of the above method the plaque rupture or superficial erosion leads to acute thrombosis, vascular occlusion, stroke, tissue infarction, or other acute vascular disease or condition.

In a first embodiment of the above methods, the compound is 2β-(2-diethylaminoethoxy)-androstenone (U18666A). In one embodiment, the compound, when administered to the subject, is at a blood concentration of from about 30 nM to about 120 nM. In another embodiment, the compound, when administered to the subject, is at a blood concentration of about 70 nM.

In a second embodiment, the compound is imipramine. In one embodiment, the compound, when administered to the subject, is at a blood concentration of from about 2 μM to about 20 μM. In another embodiment, the compound, when administered to the subject, is at a blood concentration of about 8 μM.

In a third embodiment, the intracellular cholesterol storage site is a lysosome, a recycling endosome, a sorting endosome, or a late endosome.

In a fourth embodiment, the cells are macrophage cells, endothelial cells, smooth muscle cells, T cells, or dendritic cells.

In a final embodiment, the subject is a mammal. In one embodiment, the mammal is a human.

This invention provides an article of manufacture comprising packaging material and an amphiphilic compound, wherein the compound inhibits the intracellular transport of cholesterol from an intracellular cholesterol storage site to the endoplasmic reticulum in cells, and the packaging material comprises a label indicating that the compound is intended for use in inhibiting macrophage death in a subject having, or at increased risk for developing, cardiovascular disease.

This invention also provides an article of manufacture comprising packaging material and an amphiphilic compound, wherein the compound inhibits the intracellular transport of cholesterol from an intracellular cholesterol storage site to the endoplasmic reticulum in cells, and the packaging material comprises a label indicating that the compound is intended for use in inhibiting atherosclerotic lesional complications in a subject having, or at increased risk for developing, cardiovascular disease.

In one embodiment of the above articles, the cells are macrophage cells, endothelial cells, smooth muscle cells, T cells, or dendritic cells.

In another embodiment, the compound is U18666A or a pharmaceutically acceptable salt thereof.

In a further embodiment, the compound is imipramine or a pharmaceutically acceptable salt thereof.

In a final embodiment, the subject is a human.

This invention further provides an article of manufacture comprising packaging material and an amphiphilic compound, wherein the compound inhibits the intracellular transport of cholesterol within cells, wherein the transport is from an intracellular cholesterol storage site to the endoplasmic reticulum, wherein the packaging material comprises a label indicating that the compound is intended for use in inhibiting necrosis, plaque rupture and/or superficial erosion in a subject having, or at increased risk for developing cardiovascular disease.

In one embodiment of the above article, the cells are macrophage cells, endothelial cells, smooth muscle cells, T cells, or dendritic cells.

In another embodiment, the compound is U18666A or a pharmaceutically acceptable salt thereof.

In a further embodiment, the compound is imipramine or a pharmaceutically acceptable salt thereof.

In a final embodiment, the subject is a human.

Other features and advantages of this invention will be apparent from the specification and claims which describe the invention.

This invention is illustrated in the Experimental Details section which follows. This section is set forth to aid in an understanding of the invention but is not intended to, and should not be construed to, limit in any way the invention as set forth in the claims which follow thereafter.

Experimental Details

First Series of Experiments

Synopsis

Necrotic areas of advanced atheromata are thought to play an important role in the acute clinical events associated with atherosclerotic vascular disease. Previous studies have suggested that macrophage death, perhaps caused by excess cellular FC, may contribute to the formation of these necrotic areas.

Next, we examined advanced atherosclerotic lesions in apolipoprotein E knockout (E0) mice, a model of atherosclerosis, in the absence or presence of the NPC1 mutation. In the lesions of E0 mice, there were many areas that were acellular, rich in FC but not cholesteryl esters, and, most importantly, contained macrophage proteins (i.e., “debris”). Remarkably, these necrotic areas were decreased by ˜50% (p=0.00001) in NPC1/E0 lesions whereas total atherosclerotic lesion area was decreased by only −20% in NPC1/E0 mice (p=0.05). In summary, we have shown that a partial deficiency of the npcl protein leads to a marked resistance to FC-mediated macrophage death in culture and to a selective decrease in necrotic areas in advanced atherosclerotic lesions in vivo despite only a minimal decrease in total lesion area.

Introduction

Although cell cultures studies have suggested potentially important ideas related to inducers and mechanisms of macrophage death, little is known about the factors that influence the development of lesional necrosis in vivo. We reasoned that an in-vivo model might already exist to begin to explore some of these ideas. This model, the Niemann-Pick C(NPC) mouse, like humans with NPC disease, has a mutation in a protein called npcl that results in a block of FC transport from lysosomes to peripheral cellular sites (19, 20, 21, 22). According to the ideas described herein, this specific molecular genetic defect might be expected to protect cells from FC-mediated cytotoxicity. In the first set of experiments, wild-type and heterozygous NPC(NPC1) peritoneal macrophages were loaded with FC by incubation for 24 h with acetyl-LDL plus an acyl-CoA:cholesterol acyltransferase inhibitor. The percentage of dead cells under these conditions was 29.7±4.1% for wild-type macrophages but only 8.3±1.0% of NPC1 macrophages. These findings show that a specific gene/protein alteration is associated with a selective reduction in atherosclerotic lesional necrosis.

### EXAMPLE 1

Heterozygous Deficiency of the Npc1 Protein is Associated with a Marked Resistance to Free Cholesterol-Induced Macrophage Death in Culture and to a Selective Decrease In Atherosclerotic Lesional Necrosis In Vivo

Materials & Methods

Materials

The Falcon tissue culture plasticware used in these studies was purchased from Fisher Scientific Co. (Springfield, N.J.).

Tissue culture media and other tissue culture reagents were obtained from GIBCO BRL. Fetal bovine serum (FBS) was obtained from Hyclone Laboratories (Logan, Utah) and was heat-inactivated for 1 h at 65° C. (HI-FBS). Compound 58035 (3-[decyldimethylsilyl]-N-[2-(4-mrthylphenyl)-1-phenylethyl]propanamide (23), an inhibitor of acyl-CoA:cholesterol acyltransferase (ACAT), was generously provided by Dr. John Heider of Sandoz, Inc. (East Hanover, N.J.); a 10 mg/ml stock solution was prepared in dimethyl sulfoxide, and the final dimethyl sulfoxide concentration in both treated and control cells was 0.05%. All other chemicals and reagents were from Sigma, and all organic solvents were from Fisher Scientific Co.

Mice

Balb/C mice heterozygous for the NPC mutation (NPC1) were obtained from Dr. Peter Pentchev (National Institutes of Health). These mice were backcrossed into the C57BL/6 background for four generations and then bred into the E0/C57BL/6 background for an additional generation.

Matings of NPC1/E0×NPC1/E0 were used to generate the E0 and NPC1/E0 mice used in this study. After weaning, the mice were placed on a high-cholesterol diet and sacrificed at 25 weeks of age for atherosclerotic lesion studies (below).

Harvesting and Culturing Mouse Peritoneal Macrophages

Mouse peritoneal macrophages were harvested from the peritoneum of mice 3 days after the intraperitoneal injection of 40 μg of concanavalin A in 0.5 ml of PBS (24).

The cells were plated in 22-mm dishes in Dulbecco's modified Eagle's medium (DMEM) containing 10% (v/v) FBS, 20% (v/v) L-cell conditioned medium (LCM), penicillin (100 U/ml), streptomycin (100 μg/ml), and glutamine (292 μg/ml) and then incubated at 37° C. in an atmosphere containing 5% CO₂. When the cells were 70-80% confluent, they were used for the studies described below.

FC-Loading and Cell Death Assay

Monolayers of peritoneal macrophages were washed three times with warm PBS and incubated for the indicated times in 0.5 ml of DMEM/0.2% BSA (w/v) alone or containing 10 μg acetyl-LDL/ml plus 10 μg of compound 58035/ml as previously described (25). At the end of the incubation period, the cells were assayed for cell death by permeability to the fluorescent dye propidium iodide (26). After staining with propidium iodide, the cells were viewed by fluorescence microscopy, and 15 fields of cells for each conditions (˜2000 cells) were counted to determine the percentage of propidium iodide-positive cells.

Plasma Lipid and Lipoprotein Assays Preparation and Staining of Histological Sections

Hearts from E0 and NPC1/E0 mice (above) were perfused, embedded in optimum-cutting-temperature (OCT) compound (Sakura Finetek, Torrance, Calif.), snap-frozen in ethanol-dry-ice bath and stored at −70° C. Multiple 8-μm-thick sections of murine, rabbit, and human aorta were cut on a cryostat, placed on poly-L-lysine-coated glass slides, and fixed in 10% buffered formalin for 5 min at room temperature. The sections were air-dried for 10-15 min, washed in phosphate-buffered saline (PBS) containing 0.1% Triton X-100 for 20 min, and rinsed in PBS for 5 min in PBS at room temperature. The sections were then preincubated with 2% normal serum in PBS for 1 h at room temperature. Next, the sections were incubated with 2% donkey serum. After the sections were washed in PBS for 5 min, the bound primary antibody was visualized using biotinylated secondary antibody followed by streptavidin peroxidase (Vectastain Elite ABC-peroxidase kit; Vector Laboratories Inc., Burlingame, Calif.) and 3,3′-diaminobenzidine. The sections were counterstained with hematoxylin, rinsed, mounted in permount, and viewed with an Olympus IX 70 inverted microscope using a 20× objective.

Quantification of Total Atherosclerotic Lesion Area and of Necrotic Area

On the day of analysis, food was removed from the cages in the morning, and the mice were fasted for 7 h. The animals were then anesthetized, and blood was withdrawn by cardiac puncture. The heart and vascular system was perfused with PBS in situ, and then the heart and proximal aorta were surgically removed, embedded in O.C.T. compound (Sakura Finetek), snap-frozen in an ethanol-dry ice bath, and stored at −70° C. Sections of proximal aorta, 10-μm thick, were cut at −20° C. using a Microm Microtome Cryostat HM 505 E, placed on poly-L-lysine-coated slides (Fisher Scientific, Springfield, N.J.). The sections were then fixed in 10% buffered formalin for 5 min at room temperature and air-dried for 10 min. Serial sections were stained with Harris Hematoxylin for nuclei. Intimal lesional area (area between the internal elastic lamina to the lumen), acellular areas (i.e., negative for Hematoxylin-positive nuclei), and TUNEL (terminal deoxynucleotidyl transferase end-labeling)-positive areas were quantified by blinded observers using a Nikon Labophot-2 microscope equipped with a Sony CCD-Iris/RGB color video camera attached to a computerized imaging system using Image-Pro Plus 3.0 software. For the TUNEL technique, cryostat sections were air dried and subsequently fixed by 4% buffered formalin.

We used the stringent TUNEL method described by Kockx et al. (Kockx, M. M., Muhring, J., Knaapen, M. W. & De Meyer, G. R. (1998) Am. J. Pathol. 152: 885-888), which avoids non-specific labeling due to active RNA synthesis.

Statistics

Results are given as means ±S.E.M. For comparisons between a single experimental group and a control, the unpaired, two-tailed t-test was used.

Results

Macrophages from Heterozygous NPC Mice are Resistant to FC-Mediated Cell Death

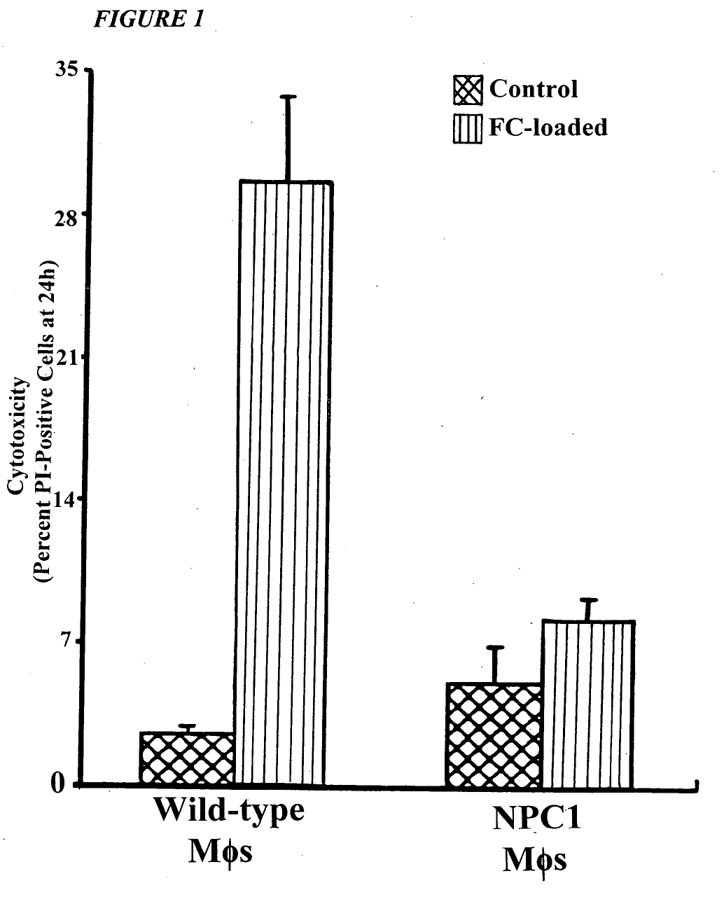

To test the idea that a molecular genetic alteration in peripheral cholesterol transport would protect macrophages from FC-mediated cytotoxicity, peritoneal macrophages from wild-type and Niemann-Pick C(NPC) mice (21,22) were loaded with FC for 12 h by incubation with acetylated LDL plus an inhibitor of cholesterol esterification (15). After 12 h of loading, the wild-type macrophages became rounded and started to detach (leading to 30% loss of attached cellular protein), whereas both the NPC1 and NPC0 macrophages were well-spread and remained attached to the plate (no loss of attached cellular protein) (data not displayed). We next compared wild-type and NPC1 macrophages using a more prolonged (24-h) FC-loading protocol. In addition, we employed a more quantitative measurement of cytotoxicity, namely, permeability to the fluorescent compound propidium iodide (PI) (26). Whereas a substantial percentage of the wild-type Mφs stained with PI as expected, the NPC1 macrophages remained mostly PI-impermeable (FIG. 1). Thus; even a partial defect in FC transport markedly protects macrophages from the toxic effects of prolonged FC loading.

To determine if NPC1 macrophages were protected from other inducers of death, we compared these cells with wild-type macrophages for their susceptibility to oxidized LDL-induced and serum withdrawal-induced death.

Characterization of Areas of Cell Death in the Atherosclerotic Lesions of E0 Mice

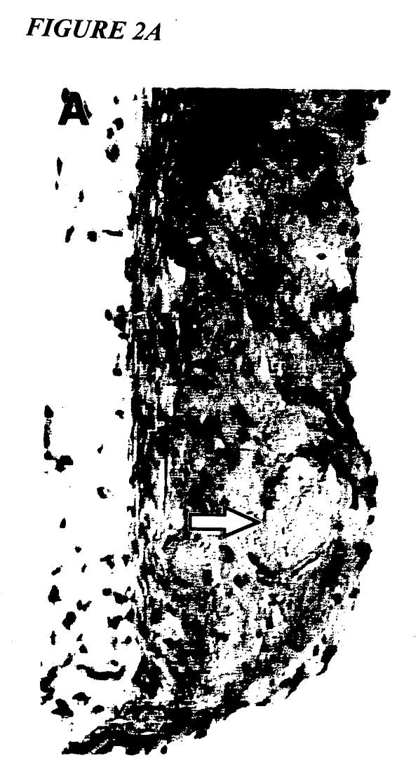

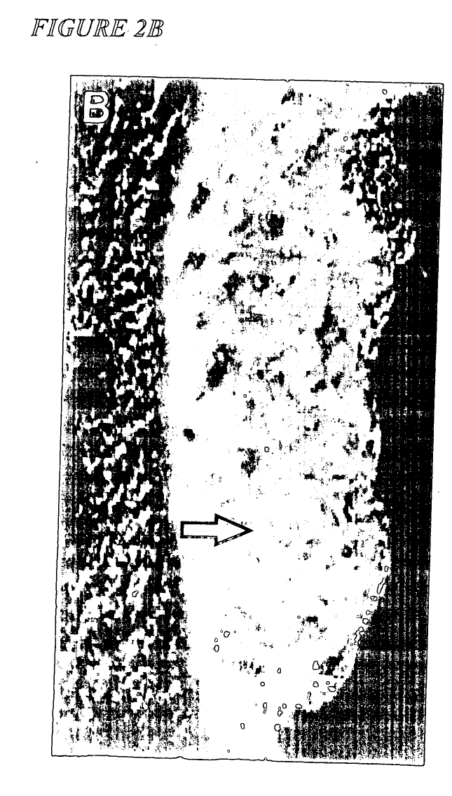

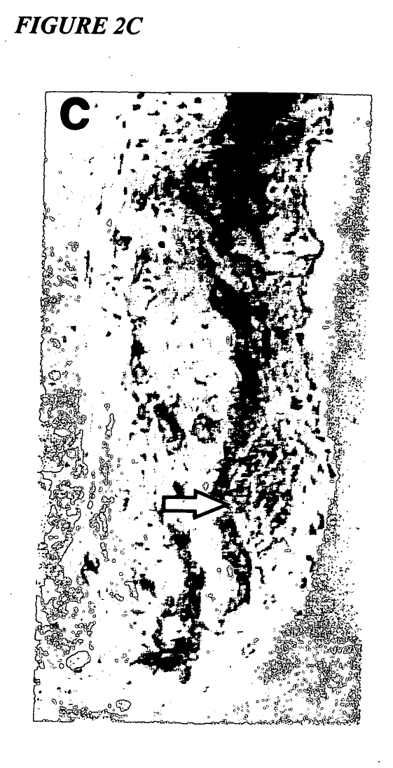

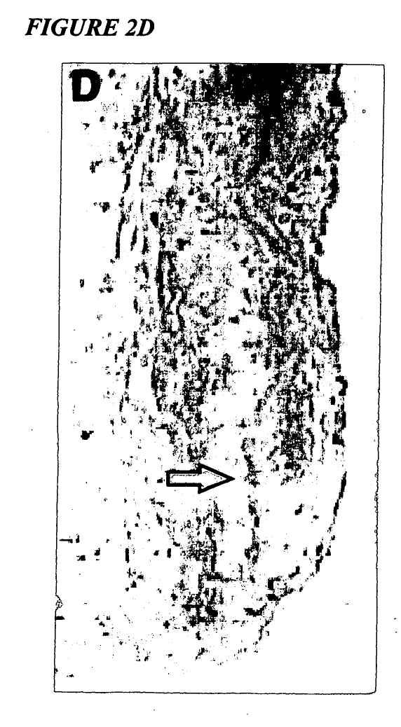

Mice engineered to lack apolipoprotein E (E0 mice) develop extensive atherosclerotic lesions with areas of necrosis (27,28). In preparation for experiments designed to look at the influence of the NPC mutation on lesional cell death (below), we characterized in detail the necrotic-appearing areas in advanced lesions of E0 mice. As shown in FIG. 2A, raised lesions from the proximal aorta of 25-week-old E0 mice contained acellular areas situated beneath a layer of endothelial and intimal cells (the arrow in FIGS. 2A-2D depicts one of these areas). Using a stain for collagen, we focused on acellular areas that were not simply dense fibrous scars (not shown). Next, because extracellular FC accumulation is a property of necrotic areas of advanced atherosclerotic lesions (1, 2, 29), we stained the lesions with filipin, a fluorescent dye that binds FC (29). As demonstrated in FIG. 2B, most of the acellular areas, as well many of the cellular areas of the intima, bound filipin, whereas the outer layer of the lesion bound no filipin. Importantly, the acellular areas of E0 lesions stained only weakly with the neutral lipid dye Oil Red 0 compared with the cholesteryl ester-rich foam cell areas (see below), indicating that the acellular areas were richer in FC than cholesteryl esters. To determine if these acellular areas might represent sites of macrophage death, we looked for the presence macrophage proteins (i.e., debris). FIG. 2C shows the result obtained when the section was immunostained for the macrophage type A scavenger receptor; FIG. 2D is the control using a nonimmune primary antibody. Remarkably, many of the acellular regions stain for this macrophage protein.

Similar results were obtained using an antibody directed against the macrophage protein Mac-3 (not shown). Thus, we have demonstrated that the advanced lesions of E0 mice contain acellular areas that stain for both FC and macrophage proteins, suggesting that these areas are, indeed, necrotic areas containing the debris of macrophages.

Analysis of the Lesions of NPC1/E0 Mice

E0 mice (in the C57BL/6 background) and NPC1 mice backcrossed for five generations to the E0/C57BL/6 background (NPC1/E0 mice) were fed a high-cholesterol (“Western”) diet for 25 weeks. Both groups of mice appeared normal, and their weights at the end of the 25-week period were not statistically different (not shown).

The plasma cholesterol and phospholipid values were also not statistically different between the two groups (FIG. 3A). Similarly, the gel-filtration profiles of the plasma lipoproteins were very similar (FIG. 3B); neither the increase in the VLDL peak nor the decrease in the LDL peak in the NPC1/E0 plasma shown in this figure was reproducible in repeated experiments.

As shown in FIG. 4A, total lesion area in the proximal aorta of NPC1/E0 mice (n=9) was decreased ˜20% compared with E0 mice (n=26; p=0.05). Despite this modest affect on lesion size, the necrotic areas of the NPC1/E0 lesions, as defined using some of the criteria described above, was decreased by −50% (FIG. 4B; p=0.00001).

Therefore, even when the data were expressed as percent necrotic area, there was a substantial, highly statistically significant difference between the two groups of mice (FIG. 4C). A separate analysis of male and female mice showed the decrease in necrotic area in NPC1 mice was not gender-specific (data not shown); in fact, NPC1/E0 male mice (n=6) had a 45% decrease in necrotic area compared with male E0 mice (n=12; p=0.0002) despite no statistically significant difference in total lesion size.

An example of a histological section from each type of mouse is shown in FIGS. 5A and 5B. In FIG. 5A the left panel shows extensive acellular areas in the lesion of an E0 mouse, and the right panel demonstrates that these acellular areas stained more weakly for Oil Red 0 than the foam cell areas, as described above. In striking contrast, the lesion of the NPC1/E0 mice shown in FIG. 5B (left panel) is more cellular and, as expected for a foam cell-rich lesion, is more Oil Red O-positive (right panel) Thus, the proximal aortic lesions of 25-week-old NPC1/E0 mice have less extensive necrotic area development than those of E0 mice.

Discussion

The data in this report reveal two important properties related to the NPC mutation. First, macrophages from mice with only a heterozygous defect in the npc protein show a marked resistance to death resulting from FC loading.

These macrophages are not resistant to other inducers of cell death, and so we presume the protection is related specifically to FC-mediated toxicity. Investigators have postulated that excess FC kills cells via inhibition of certain critical plasma membrane enzymes by a high FC:phospholipid ratio in the vicinity of these molecules (9, 16, 17). At a normal FC:phospholipid ratio, membranes contain areas of phospholipid “packing defects” that provide “space” for integral membrane proteins to undergo conformational changes. In the presence of excess FC, however, these packing defects diminish, which restricts the conformational freedom of membrane proteins and inhibits their ability to function properly (17).

Decreases in function of critical membrane proteins would then lead to cell sickness and death. FC loading may also lead to mitochondrial dysfunction, another trigger in cell death (30), perhaps by saturation of mitochondrial membranes with excess FC. Recall that FC is known to be trafficked to the mitochondria in several cell types, including macrophages (31, 32). Kellner-Weibel et al. (18) showed that amphipathic amines, which partially block FC transport out of lysosomes, prevent FC-mediated toxicity in macrophages, which is consistent with the concept that FC transport to the plasma membrane and possibly mitochondria is essential for the death response. The data in this report demonstrate this point without the use of these drugs, which may have other effects on cells, and show that the protection is still marked with only a partial defect in FC transport (33). Thus, in heterozygous NPC macrophages, a critical FC:phospholipid ratio threshold may never be reached under the conditions of our experiments.

The second major finding in this report is the effect of the NPC mutation on the characteristics of atherosclerotic lesions of E0 mice. Under conditions of similar plasma cholesterol levels and lipoprotein profiles, lesion size per se was only minimally affected, but the area of regions with the characteristics of necrosis was substantially decreased in the lesions of NPC1/E0 mice. As mentioned in the Introduction, there is evidence that macrophage death contributes to the development of lesional necrotic areas, which have been called “a graveyard of dead macrophages” (34). For example, necrotic areas of human lesions have been shown to contain macrophage proteins (4,6), consistent with our data with E0 mice presented here (FIGS. 2A-2D), and dying macrophages are often found in the vicinity of necrotic areas of lesions (1,2,7). This information, together with the increase in cellularity of the E0/NPC1 lesions (FIGS. 5A and 5B), suggests that at least one reason for the decrease in necrotic area in these lesions is a decrease in macrophage death. If so, it is tempting to speculate that the decrease in lesional macrophage death is related to the marked resistance of NPC1 macrophages to FC-induced death (FIG. 1). Indeed, macrophages in advanced lesions have been shown to accumulate large amounts of FC (10-13) which is consistent with our filipin-staining data in FIG. 2B, and so FC-induced cytotoxicity is a plausible mechanism for lesional macrophage death (16). The further testing of this hypothesis will be a focus of our future work in this area.

An issue not addressed in this study is whether FC-mediated death is due to apoptosis or necrosis (35). While the distinction between these two modes of death may not always be clear (36,37), Rothblat and colleagues (18) have presented preliminary evidence that FC loading of macrophages results in the appearance of some apoptotic features in the cells. Using a variety of assays, we have recently shown that FC loading leads to an early apoptotic response followed by later necrotic changes (Yao et al., manuscript in preparation). Because necrotic as well as apoptotic features are decreased in FC-loaded macrophages from NPC1 mice (data not shown), we conclude that normal peripheral FC transport is required for both forms of death. Of note, macrophage death in atherosclerotic lesions shows features of both apoptosis and necrosis (1).

While more studies are needed to mechanistically link the cell-culture and in-vivo data reported herein, the significance of our findings lies in the potential clinical importance of the lesional necrosis in atherosclerotic vascular disease. Atherosclerotic lesions rich in necrotic areas are often referred to as “culprit” lesions because they are susceptible to plaque rupture, which precipitates acute thrombosis and thus acute ischemic events (3). In this regard, it is likely that dying cells release or expose plaque-destabilizing enzymes (e.g., lysosomal proteases and matrix metalloproteinases) and pro-coagulant/thrombogenic molecules (e.g., tissue factor and phosphatidylserine) and thus contribute to plaque rupture and acute thrombosis (7). Indeed, Bauriedel et al. (5) have reported that atherectomy specimens from patients with unstable angina have approximately twice the number of dead intimal cells compared with specimens from patients with stable angina. Having revealed in this report a specific gene/protein alteration that results in a selective decrease in necrotic area formation, we hope to gain further insight into this critical lesional event. On a specific note, the findings reported herein raise the interesting issue as to whether humans heterozygous for the NPC mutation have a lower incidence of acute ischemic events compared to individuals without this mutation.

### EXAMPLE 2

By way of brief introduction, there is good evidence that acute clinical vascular events result from plaque rupture and that macrophage death in lesions contributes to plaque rupture. One of the causes of lesional macrophage death is likely to be excess cholesterol accumulation in the cell. We have shown that in order for excess cholesterol to kill macrophages, it must be transported from lysosomes to the plasma membrane and other peripheral sites. Pharmacologic or genetic blockage of this transport process has been shown to protect macrophages in culture from cholesterol-induced death, and we have evidence that this transport blockage also prevents macrophage death in lesions in vivo.

In essence, therefore, the invention provides a method for treating a subject suffering from advanced atherosclerosis, both before the occurrence of acute clinical events (i.e., primary prevention) and after such events (i.e., secondary prevention). This would comprise administering an inhibitor of intracellular cholesterol transport to the subject to prevent lesional macrophage death. Several known compounds, such as progesterone and various amphipathic amines, are already known to do this in cultured macrophages in vitro. In addition, the invention would provide a method for screening new inhibitors of intracellular cholesterol transport. This method would comprise the following high-throughput screening assay: (a) adding a library of compounds or derivatives of those compounds to monolayers of cultured macrophages in multi-well dishes; (b) adding one of several available toxins, such as amphotericin B, that kills cells only if the content of cholesterol in the plasma membrane is above a certain level; (c) staining dead cells with one of several available colorometric (e.g., Trypan Blue) or fluorescent (e.g., propidium iodide) dyes that do not stain live cells; and (d) colorometric or fluorescent identification of non-staining cells (i.e., those cells that survived because they have been exposed to an inhibitor of cholesterol transport to the plasma membrane). The cellular target of such inhibitors might be the protein npcl or other protein or lipid targets in the cell that may be critical for transport of cholesterol to the plasma membrane.

It should be noted that this invention is not limited to the particular embodiments described herein, but that various changes and modifications may be made without departing from the spirit and scope of this novel concept as defined by the claims which follow.

References for First Series of Experiments

1. Mitchinson, M. J., S. J. Hardwick, and M. R. Bennett. 1996. Cell death in atherosclerotic plaques. Curr. Opin. Lipidol. 7:324-329.

2. Guyton, J. R. and K. F. Klemp. 1996. Development of the lipid-rich core in human atherosclerosis. Arterioscler. Thromb. Vasc. Biol. 16:4-11.

3. Fuster, V., L. Badimon, J. J. Badimon, and J. H. Chesebro. 1992. The pathogenesis of coronary artery disease and the acute coronary syndromes. N. Eng. J. Med. 326:242-250.

4. Ball, R. Y., E. C. Stowers, J. H. Burton, N. R. Cary, J. N. Skepper, and M. J. Mitchinson. 1995. Evidence that the death of macrophage foam cells contributes to the lipid core of atheroma. Atherosclerosis 114:45-54.

5. Bauriedel, G. I. Schmucking, R. Hutter, and T. Schmidt. 1999. Increased apoptotic and necrotic cell death in human coronary plaques with unstable versus stable angina. Circulation 96:I-190(Abstr.)

6. Berberian, P. A., W. Myers, M. Tytell, V. Challa, and M. G. Bond. 1990. Immunohistochemical localization of heat shock protein—70 in normal-appearing and atherosclerotic specimens of human arteries. Am. J. Pathol. 136:71-80.

7. Libby, P. and S. K. Clinton. 1993. The role of macrophages in atherogenesis. Curr. Opin. Lipidol. 4:355-363.

8. Lopes-Virella, M. F. and G. Virella. 1992. Immune mechanisms of atherosclerosis in diabetes mellitus. Diabetes 41:Suppl 2:86-91.

9. Tabas, I. 1997. Free cholesterol-induced cytotoxicity. A possible contributing factor to macrophage foam cell necrosis in advanced atherosclerotic lesions. Trends Cardiovasc. Med. 7:256-263.

10. Lundberg, B. 1985. Chemical composition and physical state of lipid deposits in atherosclerosis. Atherosclerosis 56:93-110.

11. Small, D. M., M. G. Bond, D. Waugh, M. Prack, and J. K. Sawyer. 1984. Physicochemical and histological changes in the arterial wall of nonhuman primates during progression and regression of atherosclerosis. J. Clin. Invest. 73:1590-1605.

12. Rapp, J. H., W. E. Connor, D. S. Lin, T. Inahara, and J. M. Porter. 1983. Lipids of human atherosclerotic plaques and xanthomas: clues to the mechanism of plaque progression. J. Lipid Res. 24:1329-1335.

13. Shio, H., N.J. Haley, and S. Fowler. 1979. Characterization of lipid-laden aortic cells from cholesterol-fed rabbits. III. Intracellular localization of cholesterol and cholesteryl ester. Lab. Invest. 41:160-167.

14. Warner, G. J., G. Stoudt, M. Bamberger, W. J. Johnson, and G. H. Rothblat. 1995. Cell toxicity induced by inhibition of acyl coenzyme A: cholesterol acyltransferase and accumulation of unesterified cholesterol. J. Biol. Chem. 270:5772-5778.

15. Tabas, I., S. Marathe, G. A. Keesler, N. Beatini, and Y. Shiratori. 1996. Evidence that the initial up-regulation of phosphatidylcholine biosynthesis in free cholesterol-loaded macrophages is an adaptive response that prevents cholesterol-induced cellular necrosis. Proposed role of an eventual failure of this response in foam cell necrosis in advanced atherosclerosis. J. Biol. chem. 271:22773-22781.

16. Papahadjopoulos, D. 1974. Cholesterol and cell membrane function: a hypothesis concerning the etiology of atherosclerosis. J. Theor. Biol. 43:329-337.

17. Yeagle, P. L. 1991. Modulation of membrane function by cholesterol. Biochimie 73:1303-1310.

18. Kellner-Weibel, G., W. G. Jerome, D. M. Small, G. J. Warner, J. K. Stoltenborg, M. A. Kearney, M. H. Corjay, M. C. Phillips, and G. H. Rothblat. 1998. Effects of intracellular free cholesterol accumulation on macrophage viability: a model for foam cell death. Arterioscler. Thromb. Vasc. Biol. 18:423-431.

19. Pentchev, P. G., A. D. Boothe, H. S. Kruth, H. Weintroub, J. Stivers, and R. O. Brady. 1984. A genetic storage disorder in BALB/C mice with a metabolic block in esterification of exogenous cholesterol. J. Biol. Chem. 259:5784-5791.

20. Loftus, S. K., J. A. Morris, E. D. Carstea, J. Z. Gu, C. Cummings, A. Brown, J. Ellison, K. Ohno, M. A. Rosenfeld, D. A. Tagle, P. G. Pentchev, and W. J. Pavan. 1997. Murine model of Niemann-Pick C disease: mutation in a cholesterol homeostasis gene. Science 277:232-235.

21. Liscum, L. and J. J. Klansek. 1998. Niemann-Pick disease type C. Curr. Opin. Lipidol. 9:131-135.

22. Pentchev, P. G., E. J. Blanchette-Mackie, and L. Liscum. 1997. Biological implications of the Niemann-Pick C mutation. Sub-Cell. Biochem. 28:437-451.

23. Ross, A. C., K. J. Go, J. G. Heider, and G. H. Rothblat. 1984. Selective inhibition of acyl coenzyme A:cholesterol acyltransferase by compound 58-035. J. Biol. Chem. 259:815-819.

24. Tang, W. A. Walsh, and I. Tabas. 1999. Macrophage-targeted CTP: phosphocholine cytidylyltransferase (1-314) transgenic mice. Biochim. Biophys. Acta 1437:301-316.

25. Shiratori, Y., A. K. Okwu, and I. Tabas. 1994. Free cholesterol loading of macrophages stimulates phosphatidylcholine biosynthesis and up-regulation of CTP:phosphocholine cytidylyltransferase. J. Biol. Chem. 269:11337-11348.

26. Ross, D. D., C. C. Joneckis, J. V. Ordonez, A. M. Sisk, R. K. Wu, A. W. Hamburger, Nora RE, and R. E. Nora. 1989. Estimation of cell survival by flow cytometric quantification of fluorescein diacetate/propidium iodide viable cell number. Cancer Res. 49:3776-3782.

27. Nakashima, Y., A. S. Plump, E. W. Raines, J. L. Breslow, and R. Ross. 1994. ApoE-deficient mice develop lesions of all phases of atherosclerosis throughout the arterial tree. Arterioscler. Thromb. 14:133-140.

28. Reddick, R. L., S. H. Zhang, and N. Maeda. 1994. Atherosclerosis in mice lacking apo E. Evaluation of lesional development and progression. Arterioscler. Thromb. 14:141-147.

29. Kruth, H. S. 1985. Subendothelial accumulation of unesterified cholesterol. An early event in atherosclerotic lesion development. Atherosclerosis 57:337-341.

30. Green, D. R. 1998. Apoptotic pathways. Cell 94:695-698.

31. Bjorkhem, I., O. Andersson, U. Diczfalusy, B. Sevastik, R. J. Xiu, C. Duan, and E. Lund. 1994. Atherosclerosis and sterol 27-hydroxylase: evidence for a role of this enzyme in elimination of cholesterol from human macrophages. Proc Natl Acad Sci USA 91:8592-8596.

32. Liscum, L. and N. K. Dahl. 1992. Intracellular cholesterol transport. J. Lipid Res. 33:1239-1254.

33. Kruth, H. S., M. E. Comly, J. D. Butler, M. T. Vanier, J. K. Fink, D. A. Wenger, S. Patel, and P. G. Pentchev. 1986. Type C Niemann-Pick disease. Abnormal metabolism of low density lipoprotein in homozygous and heterozygous fibroblasts. J. Biol. Chem. 261:16769-16774.

34. Schaefer, H. E. 1981. The role of macrophages in atherosclerosis. F. Schmalze, D. Huhn, and H. E. Schaefer, editors. Disorders of the Monocyte-Macrophage System. 137-142.

35. Arends, M. J. and A. H. Wyllie. 1991. Apoptosis: mechanisms and roles in pathology. Int. Rev. Exper. Pathol. 32:223-254.

36. Majno, G. and I. Joris. 1995. Apoptosis, oncosis, and necrosis. An overview of cell death. Am. J. Pathol. 146:3-15.

37. Zhang, C.,Y. Xu, J. Gu, and S. F. Schlossman. 1998. A cell surface receptor defined by a mAb mediates a unique type of cell death similar to oncosis. Proc. Natl. Acad. Sci. USA 95:6290-6295.

Second Series of Experiments

Introduction

Cholesteryl ester-loaded macrophages, or foam cells, are prominent features of atherosclerotic lesions and play important roles in lesion progression (7,13). During atherogenesis, intimal macrophages internalize atherogenic lipoproteins, including modified forms of LDL, that have been retained in the arterial subendothelium (13,14,17). This event leads directly to esterification of cellular cholesterol by acyl-coA-cholesterol acyltransferase (ACAT), resulting in “foam cell” formation (4,14).

Foam cell formation can be prevented or reversed by a process known as cellular cholesterol efflux (15). Cholesterol efflux is the initial step of reverse cholesterol transport, a process whereby excess cholesterol in peripheral cells is delivered to the liver for excretion.

Enhancing cholesterol efflux from macrophages represents a promising strategy to promote reverse cholesterol transport and regression of atherosclerotic vascular disease.

Recently, the ATP-binding cassette transporter A1 (ABCA1) protein was shown to be an important mediator of cholesterol efflux from macrophages. Humans with full or even partial deficiency of ABCA1 have low HDL levels and increased risk for cardiovascular disease. Moreover, three reports of ABCA1 transgenic mice have shown that increased activity of ABCA1 leads to an increase in macrophage cholesterol efflux and increased reverse cholesterol transport in vivo. Thus, a potentially promising therapeutic strategy directed at atherosclerotic vascular disease is to enhance ABCA1 activity in lesional macrophages. Current strategies aimed at enhancing ABCA1 activity are directed toward increasing the cellular expression of this protein.

Macrophage death is also a prominent feature of atherosclerotic lesions (1,2,3,11) and may affect lesion progression and/or complications. For example, death of macrophages may contribute to the release of plaque-destabilizing and thrombogenic molecules in more advanced lesions. In support of this model, “necrotic” cores of advanced atheromata, which contain the debris of dead macrophages, are located in areas predisposed to plaque rupture and acute thrombosis (5). Moreover, fragments of plasma membrane shed by apoptotic lesional cells are rich in thrombogenic tissue factor activity (9). More directly, apoptotic macrophages, but not apoptotic smooth muscle cells or T cells, are greatly increased in ruptured plaques versus stable plaques (6), and atherectomy specimens from patients with unstable angina have approximately twice the number of dead intimal macrophage cells compared with specimens from patients with stable angina (2).

Among the likely causes of lesional macrophage death is intracellular accumulation of excess free cholesterol, which is known to occur in vivo. While cholesteryl ester accumulation in lesional macrophages is often emphasized, the accumulation of free cholesterol also occurs, particularly in advanced atherosclerosis (8,12).

Presumably, this occurs because progressive lipid loading of macrophages overwhelms the cell's capacity either to esterify or efflux the free cholesterol.

Hence, the free cholesterol-loaded macrophage is likely to be a critical turning point in the progression of atherosclerosis. In support of this hypothesis, lesional necrosis is a precipitating factor in plaque erosion and rupture, which in turn leads directly to acute thrombosis and acute vascular occlusion. Thus, the prevention of free cholesterol-induced macrophage death is a novel and important therapeutic strategy for the prevention of these fatal steps in atherosclerotic plaque progression.

The results described herein demonstrate that free cholesterol-loading of macrophage cells causes a reduction in ABCA1-dependent efflux activity accompanied by a proteosome-dependent decrease in ABCA1 protein levels.

Further disclosed is the surprising result that low concentrations of amphipathic amines such as imipramine and 3β-(2-diethylaminoethoxy)-androstenone (U-18666A) markedly enhance ABCA-1 mediated cholesterol efflux in free cholesterol-loaded cells. This evidence suggests that this protective effect is due to a partial inhibition of NPC1-dependent intracellular cholesterol trafficking.

Methods

Materials

Tissue culture media were from Life Technologies, Inc., and fetal bovine serum (FBS) was from Hyclone Laboratories (Logan, Utah). Tritium-labeled cholesterol and choline were from Perkin-Elmer Life Sciences, Inc. (Boston, Mass.).

Concanavalin A, ALLN, methyl-β-cyclodextrin, and imipramine were from Sigma. Compound 58035 (3-[decyldimethylsilyl]-N-[2-(4-methylphenyl)-1-phenylethyl] propanamide, may be obtained from Sandoz, Inc. (East Hanover, N.J.); a 10 mg/ml stock solution was prepared in dimethyl sulfoxide, and the final dimethyl sulfoxide concentration in both treated and control cells was 0.05%. Glyburide, sodium orthovanadate, lactacystin, and cycloheximide were from Calbiochem.

U18666A was from Biomol Research Lab, Inc. Apolipoprotein A-I (apoA-1) was from Biodesign International (Saco, ME), and rabbit anti-ABCA1 serum was from Novus (Littleton, Colo.).

Anti-β-actin, HRP-conjugated goat anti-rabbit IgG, and goat anti-mouse IgG were from Bio-Rad. LDL (d, 1.020-1.063 g/ml) and HDL₂ (d, 1.063-1.125 g/ml) from fresh human plasma were isolated by preparative ultracentrifugation. Acetyl-LDL was prepared by reaction with acetic anhydride and labeled with ³H-CE.

Harvesting and culturing mouse peritoneal macrophages The mice used in this study were wild-type C57BL6/J and BALB/c; C57BL6/J apoe KO; C57BL6/J apoE KO Nctr-npc1^{N} heterozygous; and BALB/cNctr-npc1^{N} heterozygous mice. The C57 heterozygous NPC1 apoE KO mice were produced by crossing BALB/cNctr-npc1^{N} mice (stock number 003092; Jackson Laboratory, Bar Harbor, Me.) onto C57B6/J apoE KO background for five generations. Six-ten week-old mice were injected with 0.5 ml PBS containing 40 μg of concanavalin A intraperitoneally, and the macrophages were harvested three days later by peritoneal lavage. The harvested cells were plated in cell-culture plates in Dulbecco's modified Eagle's medium (DMEM) supplemented with 10% fetal bovine serum (FBS) and 20% L-cell conditioned medium. The medium was replaced every 24 hours until the macrophages were confluent, at which point they were incubated with 50-100 μg/ml acetyl-LDL in DMEM containing 0.2% BSA with or without 10 μg/ml 58035 and/or other inhibitors.

³H-cholesterol Efflux Assay

Acetyl-LDL (800 μg) was incubated with 10 μCi ³H-cholesterol for 30 min at 37° C., followed by addition of 8 ml of DMEM, 0.2% BSA. The macrophages were incubated with this medium for 5 h, washed 3 times with PBS, and then incubated with DMEM, 0.2% BSA for 15 min at 37° C. After washing with PBS, the macrophages were incubated with DMEM, 0.2% BSA, containing either 15 μg/ml apoAI or 20 μg/ml HDL₂. At the indicated time points, 100 μl of media was removed and centrifuged for 5 min at 14,000 rpm to pellet cellular debris. The radioactivity in this fraction of media was quantitated by liquid scintillation counting. After the last time point, the remainder of the media was removed, and the cells were dissolved in 0.5 ml of 0.1 N NaOH containing 0.5% sodium dodecylsulfate (5 h at room temperature). A 100 μl-aliquot of the cell lysate was counted, and the percent efflux was calculated as (media cpm)+(cell +media cpm)×100. Total protein in the cell lysate was determined using the Bio-Rad DC protein assay kit. Note that there was no statistical difference in cellular cpm or protein between cholesteryl ester- and free cholesterol-loaded macrophages.

³H-phospholipid Efflux Assay

Macrophages were labeled with ³H-choline (5 μCi/ml) in DMEM, 10% FBS, for 24 h. After washing three times with PBS, the macrophages were incubated with 100 μg/ml acetyl-LDL+58035 in DMEM, 0.2% BSA, for 5 h. The cells were then incubated with 15 μg/ml apoA-1 in DMEM, 0.2% BSA, for the indicated time periods. ³H-choline-containing phospholipids in aliquots of the medium were extracted in chloroform:methanol (2:1, v/v), and those remaining in the cells in hexane:isopropyl alcohol (3:2, v/v). The radioactivity was measured by scintillation counting.

Whole-cell Cholesterol Esterification Assay

Macrophages were incubated in DMEM, 0.2% BSA, containing 0.1 mM ¹⁴C-oleate complexed with albumin and 3 μg/ml acetyl-LDL. At the indicated time points, the cells were washed two times with cold PBS, and the cell monolayers were extracted twice with 0.5 ml of hexane/isopropyl alcohol (3:2, v/v) for 30 min at room temperature. Whole-cell cholesterol esterification activity was assayed by determining the cellular content of cholesteryl ¹⁴C-oleate by thin-layer chromatography. The cell monolayers were dissolved in 1 ml of 0.1 N NaOH, and aliquots were assayed for protein by the Lowry method.

Biotinylation of Cell-Surface Proteins

Macrophage monolayers in 35-mm dishes were washed with ice-cold PBS 3 times and then incubated with ice-cold PBS containing 0.5 mg/ml NHS—SS-biotin (Pierce) for 30 minutes at 4° C. After washing 5 times with ice-cold PBS containing 20 mM Tris-HCl, pH 8.0, the cells were scraped into PBS and pelleted by centrifugation. The pelleted macrophages were lysed in 50 μl RIPA buffer (0.5% sodium deoxycholate, 0.1% SDS, 1% Triton X-100, 20 mM Tris, 150 mM NaCl, and 5 mM EDTA, pH 8) containing 1 mM PMSF. Ten 1 μl of the lysate were subjected directly to 4-20% gradient SDS-polyacrylamide gel electrophoresis (SDS-PAGE) for determination of total ABCA1. The rest of the cell lysate was affinity-purified to isolate biotinylated proteins. Briefly, the cell lysates were diluted to 150 μl in RIPA buffer and incubated with 50 μl immobilized streptavidin agarose (Pierce), which was pre-washed three times with RIPA buffer at 0° C. for 2 h with gentle shaking. The agarose was pelleted by centrifugation using a microcentrifuge at 5,000 rpm for 2 min; the pellet was resuspended in 1 ml RIPA buffer, and the process was repeated 5 times. The agarose was resuspended 30 μl SDS-PAGE loading buffer containing 330 mM β-mercaptoethanol at 37⁶C for 15 min and subjected to SDS-PAGE as above. ABCA1 and β-integrin were detected by Western blot using anti-ABCA1 and anti-β-integrin anti-sera. The blots were reprobed with anti-β-actin antibody, which detected no actin signal, thus verifying that no cytosolic protein was biotinylated by the procedure.

Western Blot Analysis

Peritoneal macrophages were lysed in RIPA buffer containing 1 mM PMSF. Nuclei were removed by centrifugation at 3000×g for 10 min at 4° C. Protein in the supernatants (15-30 μg of protein) was separated by electrophoresis on 4-20% gradient SDS-PAGE and electro-transferred to a 0.22-μm nitrocellulose membrane using a Bio-Rad mini transfer tank (Bio-Rad). For Western blot detection of ABCA1, anti-ABCA1 antiserum was used. Signals were detected using HRP-conjugated secondary anti-bodies (Bio-Rad) and ECL (Amersham Pharmacia Biotech). The membranes were reprobed with anti-β actin monoclonal antibody or anti-β1-integrin anti-serum for the proper internal controls. The relative intensities of the bands were determined by densitometry.

Real-time Quantitative RT-PCR

Monolayers of macrophages in 22-mm dishes were incubated for 5 h with 100 μg/ml acetyl-LDL in the absence or presence of 10 μg/ml 58035. After washing with cold PBS, the cells were lysed with 1 ml Trizol reagent to isolate total RNA. Five μg total RNA was reversed transcribed using BRL Superscript II and polyT as the primer, and PCR was conducted using 62.5 ng cDNA in the M×4000TM Multiplex Quantitative PRC system from Stratagene. The primers for the ABCA1 gene were 5′-cctcagccatgacctgccttgtag-3′ and 5′-ccgaggaagacgtggacaccttc-3′. To control for input cDNA, a β-actin primer/probe set from PE Biosystems was used. The PCR products were checked by agarose gel electrophoresis to make sure a single PCR product was obtained. A standard curve was obtained by plotting the cycle threshold versus the log of input cDNA, which was obtained from CE-loaded mouse peritoneal macrophages. Both the β-actin and ABCA-I standard curves were linear between 31.25 and 250 ng cDNA (r²=0.99 for both). The PCR reactions were set up using SYBR-Green PCR Core Reagents from Applied Biosystems. The PCR was initiated at 95° C. for 10 min, followed by 45 cycles consisting of 95° C. for 0.5 min, 56° C. for 1.5 min, and 72° C. for 1.4 min. After obtaining real time fluorescence measurements, cycle threshold values were determined. Using the standard curves in the linear range (i.e., exponential amplification phase), the quantities of ABCA-I and β-actin mRNAs were calculated. The final data are expressed as the ratio of ABCA1:β-actin mRNA.

In vivo efficacy of U18666A

LDL receptor knockout mice were fed a diet containing cholesterol and saturated fat for 12 weeks in the absence or presence of 0.75 mg/kg/d U18666A (10 mice per group).

Statistics

Results are given as means±S.E.M. (n=3); absent error bars in the figures signify S.E.M. values smaller than the graphic symbols. For the quantitative PCR measurements, triplicate values were obtained, and there was <1% variation among these values.

Results

Free cholesterol loading of macrophages leads to the dysfunction of the ABCA1 cholesterol efflux pathway In order to mimic the pathology of the cholesterol-loaded macrophage that occurs in atherosclerotic lesions, an assay was developed wherein cultured peritoneal macrophages are induced to accumulate excess cholesterol, either predominantly in the form of cholesteryl esters or in the form of free cholesterol. The relative effect of cholesteryl ester loading versus free cholesterol loading on cholesterol efflux from the cells was then determined. ApoA-I was used as an ABCA1-specific cholesterol acceptor protein in order to differentiate ABCA1-dependent from ABCA1-independent efflux in this assay.

Mouse peritoneal macrophages were incubated with tritiated cholesterol-labeled acetyl-LDL either alone, to effect predominantly cholesteryl ester loading, or in the presence of the ACAT inhibitor, 59035, for free cholesterol loading.

Cholesterol efflux was measured in the presence of either apoA-I or HDL₂, which does not bind ABCA1 and therefore serves as a measure of efflux through ABCA1-independent pathways. As shown in FIGS. 6A-6E, free cholesterol-loaded cells demonstrated a marked reduction in cholesterol efflux to apoA-I (FIG. 6A), while only modestly affecting efflux to HDL₂ (FIG. 6B). Furthermore, as shown in FIG. 6D, the free cholesterol-induced defect in efflux to ApoA-I was manifest within the first hour following cholesterol loading of the cells. These results indicate both that ABCA1 transporter activity was particularly sensitive to impairment by excessive intracellular free cholesterol and that its impairment is an early event following free cholesterol accumulation.

In order to examine the relative activity of ABCA1 in the cholesterol-loaded cells, phosphatidylcholine efflux to apoA-I was measured in both free- and cholesteryl ester-loaded macrophages. This assay is based on a model in which ABCA1-mediated cholesterol efflux is divided into two sequential steps, (i) phospholipid efflux to lipid-free apoA-I, and (ii) cholesterol efflux to these apoA-I -phospholipid complexes. Relying on this model, a defect in phospholipid efflux indicates reduced ABCA1 transporter activity.

As demonstrated in FIG. 6C, free cholesterol-loaded cells exhibited substantially reduced phosphatidylcholine efflux compared with that of cholesteryl ester-loaded cells. Furthermore, as shown in FIG. 6E, this defect was reversed by treatment of the cells with methyl-β-cyclodextrin, which removes free cholesterol. These results demonstrate that ABCA1 transporter activity is compromised in free cholesterol-loaded cells and that this defect is largely due to the free cholesterol itself.

FC-Loading of macrophages leads to a decrease in ABCA1 protein but not in ABCA1 mRNA

In order to determine whether the decrease in ABCA1 transporter activity in free cholesterol-loaded macrophages correlated with lower protein levels, lysates from cells that were cholesterol loaded for either 5 or 7 hours were analyzed for ABCA1 protein expression using standard western immunoblotting techniques. As shown in FIG. 7A, total ABCA1 protein was substantially decreased in free cholesterol-loaded cells compared with cholesteryl ester-loaded cells at both the 5 and 7 hour time points.

Normalized to β-actin, the data demonstrated a 75% decrease in ABCA1 expression at 5 h and a greater than 90% decrease at 7 h. In contrast, the cholesteryl ester-loaded cells showed a 2.4-fold increase in ABCA1 protein expression between 5 and 7 h, consistent with the induction of ABCA1 expression previously reported in response to sterol loading. As shown in FIG. 7B, the decreased expression of ABCAL in free cholesterol-loaded cells was even more pronounced in the membrane fraction.

These results indicate that the expression of ABCA1, particularly that of the membrane-associated protein, was substantially diminished in free cholesterol-loaded cells.

As shown in FIG. 8A, this decreased expression of the protein did not correlate with a reduction in ABCA1-mRNA levels. It was therefore determined whether there was reduced translation of the ABCA1 mRNA in free cholesterol-loaded cells. To do this, cycloheximide-treated cells were cholesterol-loaded and examined for ABCA1 protein expression. As shown in FIG. 8B (top and middle blot), the decrease in ABCA1 protein observed in free cholesterol-loaded cells was insensitive to cycloheximide. Together with the mRNA data, these results indicate that a post-translational mechanism is likely to be responsible for the observed decrease in ABCA1 protein levels. Consistent with this, both ALLN, an inhibitor of cysteine proteases and proteasomal degradation, and lactacystin, a specific inhibitor of proteasomal degradation, blocked the decrease in ABCA1 in free cholesterol-loaded macrophages. Inhibitors specific for the cysteine protease calpain, calpeptin (40 μM) and PD150606 (25 μM), did not affect the decrease in ABCA1 in FC-loaded macrophages (data not shown).

In summary, these results demonstrate that free cholesterol-loading of macrophages results in a substantial decrease in ABCA1 protein expression, most likely through increased proteasome-dependent degradation.

Studies with Heterozygous NPC1 Mutant Macrophages

These results thus far indicate that free cholesterol-loading leads to defective ABCA1-mediated cholesterol efflux and increased turnover of the ABCA1 protein. Since ABCA1 functions as a transporter, it was determined whether free cholesterol-loading is also associated with defects in intracellular cholesterol transport using macrophage cells from mice carrying a heterozygous deletion in the gene for NPC1.

NPC1, the protein defective in type I Niemann-Pick C disease, is required for the normal trafficking of cholesterol out of late endosomal and/or lysosomal compartments. In free cholesterol-loaded macrophages, cholesterol accumulates in perinuclear organelles, presumably late endosomes or lysosomes, and also traffics to peripheral sites, such as the plasma membrane and endoplasmic reticulum. It was previously shown that cholesterol efflux via both ABCA1-dependent and independent pathways is severely disrupted in macrophages from homozygous NPC1 knockout mice, presumably because cholesterol transport from late endosomes and/or lysosomes to the ABCA1 efflux pathway in the plasma membrane is defective.

NPC1 heterozygous macrophage cells provide a system in which cholesterol trafficking to the plasma membrane remains mostly intact while trafficking to other intracellular peripheral sites is severely compromised. It was demonstrated previously that NPC1 heterozygotes exhibit only a slight defect in cholesterol trafficking to the plasma membrane (about a 10-15% decrease compared with wild-type cells). However, as shown in FIG. 9A, trafficking to the endoplasmic reticulum was decreased by as much as 50% in these cells, consistent with the requirement for NPC1 in cholesterol transport from late endosomes and/or lysosomes.

To examine the effects of cholesterol-loading in this system, cholesterol-loaded wild-type (NPC+/+) and heterozygous mutant (NPC+/−) macrophages were assayed for efflux to apoA-I and HDL₂ as described previously. Importantly, there was no significant difference in cholesterol loading between the two genotypes. As shown in FIG. 9B, cholesterol efflux to apoA-I was markedly increased in the NPC+/−macrophages compared with NPC+/+macrophages. This efflux was effectively blocked by two inhibitors of the ABCA1 efflux pathway, glyburide and ortho-vanadate (FIG. 9D), demonstrating that the increased efflux was ABCA1-dependent. As expected from the previous data, ABCA1-independent efflux to HDL₂ was already relatively high in NPC+/+free cholesterol-loaded macrophages, and it was increased only slightly by the heterozygous NPC mutation (FIG. 9C). Thus, NPC+/−macrophages were protected from the free cholesterol-induced defect in the ABCA1-dependent efflux pathway.

It was next determined whether rescue from the defect in cholesterol efflux was accompanied by an increase in ABCA1 protein expression in the NPC+/−macrophages. Consistent with earlier results, there was an approximately 95% decrease in total ABCA1 protein and an approximately 80% decrease in cell-surface ABCA1 protein in free cholesterol-loaded NPC+/+macrophages (FIG. 10). Strikingly, NPC+/−macrophages exhibited only about a 5% decrease in total ABCA1 and a 25% decrease in cell-surface ABCA1.

Together, these data indicate that free cholesterol-loading induces degradation of ABCA1 and that the resulting defect in cholesterol efflux to apoA-I requires intact trafficking of free cholesterol to a peripheral cellular site. Furthermore, these data indicate that a partial inhibition of intracellular cholesterol trafficking offers a dramatic protective effect against free cholesterol-induced defects in ABCA1 mediated efflux.

Studies with Low-Dose Amphipathic Amines

In order to test the idea that partial disruption of cholesterol trafficking offers a protective effect to ABCA1 in free cholesterol-loaded cells, the ability of certain types of amphipathic amines, such as 2α-(2-diethlaminoethoxy)-androstenone (U18666A) and imipramine, to mimic the NPC mutant phenotype was exploited.

Efflux from free cholesterol-loaded macrophage cells was measured as described previously in the absence or presence of either U18666A or imipramine, as indicated in FIGS. 11A and 11B. Notably, both compounds exhibited a marked ability to induce cholesterol efflux to ApoA-1. Peak efflux was observed at 70 nM for U18666A (FIG. 11A), which was almost 100-fold less than the peak concentration for imipramine (FIG. 11B). At concentrations greater than 100 nM, U18666A gradually inhibited efflux, presumably due to a severe blockage of cholesterol trafficking to the plasma membrane. A similar biphasic profile was observed with imipramine. Importantly, 70 nM U18666A decreased cholesterol trafficking to the endoplasmic reticulum by about 90% decrease while trafficking to the plasma membrane was reduced by only about 10% (data not shown).

These results suggest that optimal low doses of inhibitors such as U18666A and imipramine restored ABCA1-dependent efflux in free-cholesterol loaded macrophages. As shown in FIG. 12A, this effect was sufficient to restore efflux to the levels observed in cholesteryl ester-loaded cells. In addition, while U18666A improved both ABCA1-dependent and independent efflux from free cholesterol-loaded cells, the net effect was substantially greater for ABCA1-dependent efflux (compare FIGS. 12A and 12B).

Notably, as shown in FIG. 12C, 70 nM U18666A also increased cholesterol efflux to apoA-I by about 30% in macrophages incubated for a prolonged period with acetyl-LDL without an ACAT inhibitor. These data raise the possibility that the amount of free cholesterol that naturally accumulates under these conditions may be enough to cause modest dysfunction of the ABCA1 cholesterol efflux pathway.

Consistent with its ability to restore ABCA1-mediated cholesterol efflux, U18666A also protected from the ABCA1 protein loss observed in free cholesterol-loaded cells (FIG. 13, top panel). This protective effect was particularly striking in the case of cell-surface ABCA1 protein, which decreased by only about 15% in U18666A-treated cells, compared with 60% in untreated cells (FIG. 13, bottom panel).

In vivo efficacy of U18666A in a mouse model of atherosclerosis

In order to determine if the ability of U18666A to maintain ABCA1-dependent efflux in free cholesterol-loaded cells translates into a protective effect against atheroslerosis in vi vo, the effect of low-dose U18666A on lesion development in the LDL receptor knockout mouse model was examined. LDL receptor knockout mice were fed a diet containing cholesterol and saturated fat for 12 weeks in the absence or presence of 0.75 mg/kg/d U18666A (10 mice per group). As shown in FIGS. 14A-14E, the plasma levels of both total cholesterol (FIG. 14A) and HDL (FIG. 14B) cholesterol are similar in the U18666A-treated group compared to those receiving vehicle alone. However, the U18666A treatment group exhibited a marked reduction in atherosclerotic lesion progression as measured by lesion area (FIG. 14C), acellular area (FIG. 14D), and lipid core area (FIG. 14E). Thus, these results demonstrate the feasibility of therapeutic protocols for atherosclerosis.

Discussion

The results reported herein demonstrate that ABCA1-dependent cholesterol efflux is severely disrupted by the accumulation of free cholesterol in macrophage cells. These results further demonstrate that this disruption parallels a free cholesterol-dependent degradation of the ABCA1 protein. Thus, these results suggest a novel strategy for therapeutic intervention in atherosclerosis, namely the protection of macrophage ABCA1 from free-cholesterol-induced degradation.

Lesional macrophage cells are particularly susceptible to the damaging effects of high levels of intracellular free cholesterol because they internalize large amounts of lipoprotein cholesterol by means other than the LDL receptor, such as by phagocytosis. Therefore, a number of cellular mechanisms for preventing the accumulation of free cholesterol are not available to the macrophage.

Here, it is shown that a free cholesterol-induced degradation of the ABCA1 protein is an early event in the loss of ABCA1-dependent cholesterol efflux activity in free cholesterol-loaded macrophages. It is further demonstrated that this degradation of ABCA1 is proteosome-dependent and occurs well before the appearance of overt biochemical and morphological signs of cytotoxicity, such as a drop in mitochondrial membrane potential, caspase activation, cell shape changes, and membrane permeability disruptions.

At later times, as free cholesterol continues to accumulate, other factors are likely to contribute to the disruption of ABCA1-dependent efflux. For example, alterations in the fluidity of the plasma membrane may adversely affect the transport activity of ABCA1 or decreased cellular ATP levels may contribute to the inactivation of ABCA1, whose transporter activity is ATP-dependent. However, intervention to preserve ABCA1 functionality is less likely to succeed once the cell has sustained this level of damage.

While current efforts to increase ABCA1 activity are focused primarily on increasing ABCA1 gene expression, the results herein suggest that this method will ultimately fail, since the protein will be degraded. Instead, these results point to an alternative strategy, namely the inhibition of the proteosomal degradation of ABCA1 that is demonstrated herein to be induced by excess intracellular free cholesterol.

The results herein also indicate that the triggering of ABCA1 degradation requires trafficking of cholesterol from late endosomes/lysosomes to a peripheral cellular site, perhaps the endoplasmic reticulum, but not to the plasma membrane itself. This interpretation is supported both by the results herein using the NPC1 heterozygous mutant macrophage cells and the results herein with normal macrophages treated with the amphipathic amines imipramine and U18666A. While others have also demonstrated similar effects of low-dose U18666A on cholesterol trafficking to the ER versus the plasma membrane (16), the results presented herein are the first to link this defect with both ABCA1 activity and cholesterol efflux.

Most importantly, the instant work reveals the unexpected discovery that partial inhibition of NPC1, either genetically or pharmacologically, is an effective block against free cholesterol-induced ABCA1 degradation. The surprising result that low concentrations of imipramine and U18666A markedly enhance ABCA1-mediated cholesterol efflux and ABCA1 protein expression in free cholesterol-loaded cells demonstrates that these and similar compounds have therapeutic use as agents in the treatment of atherosclerosis.

Finally, the usefulness of U18666A and related compounds for the treatment of atherosclerosis is demonstrated by the remarkable success of the instant protocol in LDL receptor knockout mice. These results demonstrate that U18666A significantly reduces lesion progression in these mice.

References For Second Series of Experiments

1. Ball, R. Y., Stowers, E. C., Burton, J. H., Cary, N. R., Skepper, J. N., and Mitchinson, M. J. (1995) Atherosclerosis 114, 45-54.

2. Bauriedel, G., Schmucking, I., Hutter, R., Luchesi, C., Welsch, U., Kandolf, R., and Luderitz, B. (1997) Z. Kardiol. 86, 902-910.

3. Berberian, P. A., Myers, W., Tytell, M., Challa, V., and Bond, M. G. (1990) Am. J. Pathol. 136, 71-80.

4. Brown, M. S. and Goldstein, J. L. (1983) Annu. Rev. Biochem. 52, 223-261.

5. Fuster, V., Badimon, L., Badimon, J. J., and Chesebro, J. H. (1992) N. Engl. J. Med. 326, 242-250.

6. Kolodgie, F. D., Narula, J., Burke, A. P., Haider, N., Farb, A., Hui-Liang, Y., Smialek, J., and Virmani, R. (2000) Am. J. Pathol. 157, 1259-1268.

7. Libby, P. and Clinton, S. K. (1993) Curr. Opin. Lipidol. 4, 355-363.

8. Lundberg, B. (1985) Atherosclerosis 56, 93-110.

9. Mallat, Z., Hugel, B., Ohan, J., Leseche, G., Freyssinet, J. M., and Tedgui, A. (1999) Circulation 99, 348-353.

10. Matsuda, K. (1994) Med. Res. Rev. 14, 271-305.

11. Mitchinson, M. J., Hardwick. S. J., and Bennett, M. R. (1996) Curr. Opin. Lipidol. 7, 324-329.

12. Rapp, J. H., Connor, W. E., Lin, D. S., Inahara, T., and Porter, J. M. (1983) J. Lipid Res. 24, 1329-1335.

13. Ross, R. (1995) Annu. Rev. Physiol. 57, 791-804.

14. Tabas, I. (2000) Biochim. Biophys. Acta 1529, 164-174.

15. Tall, A. R. (1998) Eur. Heart J. A31-A35.

16. Underwood, K. W., Andemariam, B., McWilliams, G. L., and Liscum, L. (1996) J. Lipid Res. 37, 1556-68.

17. Williams, K. J. and Tabas, I. (1995) Arterioscler. Thromb. Vasc. Biol. 15, 551-561.

Third Series of Experiments

Synopsis

Excess cellular cholesterol induces apoptosis in macrophages, an event likely to promote the progression of atherosclerosis. The cellular mechanism of cholesterol-induced apoptosis is unknown but had been thought to involve the plasma membrane. We report that the endoplasmic reticulum (ER) unfolded protein response (UPR) is activated in cholesterol-loaded macrophages, leading to expression of the cell death effector CHOP. Cholesterol loading depletes ER calcium stores, an event known to induce the UPR. ER calcium depletion, the UPR, caspase-3 activation, and apoptosis are markedly blunted by selective inhibition of cholesterol trafficking to the ER, and Chop−/− macrophages are protected from cholesterol-induced apoptosis. We propose that cholesterol trafficking to ER membranes, leading to activation of the CHOP arm of the UPR, is the key signaling step in cholesterol-induced apoptosis in macrophages.

Introduction

Cells possess a variety of homeostatic mechanisms to prevent the accumulation of excess free cholesterol (FC), including acyl-CoA:cholesterol acyltransferase (ACAT)-mediated cholesterol esterification (1,2). A highly relevant cell type in this regard is the macrophage, because macrophages accumulate FC in advanced atherosclerotic lesions, leading to macrophage apoptosis and lesion progression (2-9).

Previous studies with cultured macrophages have clearly documented activation of downstream apoptotic events by FC loading (2, 10-12). However, the proximal FC-induced events that trigger apoptosis have not yet been identified. Rothblat and colleagues recently showed that FC-induced macrophage death could be blocked by micromolar concentrations of certain amphipathic amines that are known to interfere with cholesterol trafficking from late endosomes to other cellular sites, particularly the plasma membrane (10,13). Based on these observations and previous in vitro data showing that plasma membrane proteins function poorly in lipid bilayers enriched with abnormally high concentrations of cholesterol (14), they proposed that death of FC-loaded macrophages was triggered by abnormal FC enrichment of the plasma membrane (13).

Cholesterol from the breakdown of lipoprotein particles in late endosomes is also directed to cellular sites other than the plasma membrane. These include the ER, the site of re-esterification. Because the cholesterol content of the lipid bilayer is particularly low in ER membranes (15), the function of the organelle is likely to be especially sensitive to abnormal enrichment in FC. Perturbations of ER function from many causes activate a signaling pathway referred to as the unfolded protein response (UPR) (16). The UPR leads to transcriptional activation of genes whose products promote the ER's capacity to process client proteins, synthesize phospholipids, and re-esterify sterols (17). These last two rectifying responses may play a role in lessening the burden of FC accumulation in the ER and other cell membranes (18). However, the UPR also activates signaling pathways promoting programmed cell death. The latter include the activation of a specific caspase (19), JUN N-terminal stress activated protein kinases (20,21) and the transcription factor CHOP (22,23). These observations suggested to us the possibility that FC loading of ER membranes may contribute to macrophage apoptosis.

In this study we use genetic and pharmacological tools that selectively perturb cholesterol trafficking from late endosomes to specific cellular sites to further explore the basis of FC-induced apoptosis in macrophages. Our findings reported here implicate ER-based signals in the death of FC-loaded macrophages and thus provide new insight into the relationship between cholesterol homeostasis in the ER membrane and ER function. Moreover, the data suggest a novel cellular mechanism for cholesterol-induced macrophage death in advanced atherosclerotic lesions.

Methods and Materials

Materials

The Falcon tissue culture plasticware used in these studies was purchased from Fisher Scientific Co. (Springfield, N.J.). Tissue culture media and other tissue culture reagents were obtained from GIBCO BRL. Fetal bovine serum (FBS) was obtained from Hyclone Laboratories (Logan, Utah) and was heat-inactivated for 1 h at 65° C. (HI-FBS). Compound 58035 (3-[decyldimethylsilyl]-N-[2-(4-methylphenyl)-1-phenylethyl] propanamide (47), an inhibitor of acyl-CoA:cholesterol acyltransferase (ACAT), was generously provided by Dr. John Heider, formerly of Sandoz, Inc. (East Hanover, N.J.); a 10 mg/ml stock solution was prepared in dimethyl sulfoxide, and the final dimethyl sulfoxide concentration in both treated and control cells was 0.05%.

Cholesterol (>99% pure) was obtained from Nu-chek Prep, Inc. (Elysian, Minn.). U18666A (3-beta[2-diethylaminoethoxy]androst-5-en-17-one hydrochloride) was from Biomol (Plymouth Meeting, Pa.). LDL (d, 1.020-1.063 g/ml) from fresh human plasma was isolated by preparative ultracentrifugation. Acetyl-LDL was prepared by reaction with acetic anhydride (48). All other chemicals and reagents, including androstenediol, Streptomyces cholesterol oxidase, methyl-α-cyclodextrin, concanavalin A, A32187, thapsigargin, tunicamycin, and anti-α-actin monoclonal antibody, were from Sigma, and all organic solvents were from Fisher Scientific Co. The methyl-β- cyclodextrin was saturated with cholesterol as previously described (30). Anti-GADD153 (CHOP) was from Santa Cruz Biotechnology. Antibodies against XBP-1, ATF4, PERK and IRE1α were made as described (35,36,38). HRP-conjugated goat anti-rabbit IgG and goat anti-mouse IgG were from Bio-Rad, and rabbit anti-lamin B polyclonal antiserum was a generous gift from Dr. Eugene Marcantonio (Department of Pathology, Columbia University).

Mice

Apoe−/− mice on the C57BL/6 background were placed after weaning on a high cholesterol diet containing 21% anhydrous milk fat and 0.15% cholesterol (“Westerntype” diet; Harlan-Teklad, Indianapolis, Ind.) for the indicated times. Chop−/− mice on the FVB/N background and Perk−/− mice on the Swiss-Webster background were created as previously described (22, 33). For experiments involving these mice, control macrophages were obtained from wild-type siblings.

FC loading and cell death assays of mouse peritoneal macrophages

Peritoneal macrophages from adult female C57BL/6 mice and all mutant mice used in this study were harvested three days after the intraperitoneal injection of 40/g of concanavalin A in 0.5 ml of PBS. The cells were incubated in Dulbecco's modified Eagle's medium (DMEM) supplemented with 10% fetal bovine serum (FBS) and 20% L-cell conditioned medium. The medium was replaced every 24 hours until the macrophages were confluent. On the day of the experiment, the cells were washed three times with warm PBS and incubated as indicated in the Description of Figures for FIGS. 15A-20E. Most importantly, FC-loading of wild type and mutant macrophages was effected by incubating the cells with 100 μg/ml acetyl-LDL in the presence of 10 μg/ml 58035, which inhibits ACAT-mediated cholesterol esterification (11). At the end of the incubation period, the macrophages were assayed for early-mid-stage apoptosis (i.e., phosphatidylserine externalization) by staining with Alexa-488-labeled annexin V and for late-stage apoptosis (i.e., increased membrane permeability) by staining with propidium iodide (PI), as previously described (11). Cells were immediately viewed with an Olympus IX-70 inverted fluorescence microscope, and 3-6 representative fields (˜1000 cells) for each condition were counted for the number of annexin-positive, PI-positive, and total cells. In other experiments, cell or nuclear preparations were subjected to immunoblot analysis as described below.

Activated caspase-3 in macrophages was detected by immunofluorescence microscopy using an antibody that specifically recognizes the active form of this caspase (Apo-Active 3 kit from Cell Technology).

Whole-cell Cholesterol Esterification Assay

Macrophages were incubated for 5 h with DMEM, 0.2% BSA containing 50 μg/ml [³H]cholesterol-labeled acetyl-LDL alone or containing the indicated compounds. Cellular lipids were extracted twice with 0.5 ml of hexane:isopropanol (3:2, v/v), and the cellular content of [³H]cholesteryl ester was determined using thin-layer chromatography (49). The lipid-extracted cell monolayer was dissolved in 1 ml 1N NaOH and assayed for protein content using the method of Lowry et al. (50).

Cholesterol Oxidase Assay

Macrophages were fixed with 1% glutaraldehyde for 10 min and then incubated for 30 min at 37° C. with 2 U/ml Streptomyces cholesterol oxidase. Cellular lipids were extracted twice with 0.5 ml of hexane:isopropanol (3:2).

Cholesterol and.cholestenone mass in these extracts were determined by gas-liquid chromatography using β-sitosterol as an internal standard. The lipid-extracted cell monolayer was dissolved in 1 ml 1N NaOH and assayed for protein content using the method of Lowry et al. (50).

Immunoblot Analysis

Immunoprecipitation and immunoblot detection for IRE1α and PERK were conducted as described by Harding et al. (35). Immunoblot analyses of XBP-1, ATF4, and CHOP were carried out as described previously (36,38) with minor modifications. Briefly, cells were lysed in RIPA buffer to prepare whole-cell lysates or to prepare nuclei by centrifugation. The whole-cell lysates or nuclei were resuspended in 2×SDS-PAGE loading buffer and incubated at 95° C. for 10 min. Whole cell (100 μg) or nuclear lysates (20 μg) were electrophoresed on 4-20% gradient SDS-PAGE gels and electrotransferred to 0.22-μm nitrocellulose membrane using a Bio-Rad mini-transfer tank. After incubation with primary antibodies, the protein bands were detected with HRP-conjugated secondary antibodies (Bio-Rad) and ECL (Amersham Pharmacia Biotech). The membranes were reprobed with an anti-β-actin monoclonal antibody or an anti-lamin B serum to control for differences in loading.

Assay of ER Calcium Pools

Untreated or cholesterol-loaded macrophages on 25-mm coverslip-containing microscopy dishes were loaded with 4 μM Fura-2 μM (Molecular Probes) and 0.08% Pluronic F-127 in Hank's buffered saline solution (HBSS) at room temperature for 30 min. The monolayers were then washed twice with HBSS, incubated with HBSS for an additional 30 min and mounted on the stage of an inverted Nikon diaphot microscope equipped with a 40× objective. Sulfinpyrazone (250 μM) was included in all solutions to prevent excretion of the Fura-2 by the macrophages. Fluorescence images (510-nm emission following alternate 340-nm and 380-nm excitation) were collected through a charge-coupled device camera (Photon Technology International), and the 340:380 ratio of individual cells in these images was calculated.

In-Si tu Hybridization

The 470-bp BamHI-NheI fragment of CHOP was subcloned into the BamHI and XbaI sites of the pCR11-TOPO vector (Invitrogen). The resulting plasmid was transcribed in vitro using the DIG-RNA Labeling kit (SP6/T7; Roche Molecular Biochemicals) to generate sense and anti-sense Dig-ribo probes. Proximal aortae from Apoe−/− mice fed the Western-type diet for 13 weeks were fixed in 4% paraformaldehyde in PBS at 4° C. overnight and then submerged in 30% (w/v) sucrose in PBS for 48 hours at 4° C. The tissue was then embedded in OCT, cut into 10-μm sections, and mounted on Superfrost/plus slides (Fisher Scientific). The sections were then fixed in 4% paraformaldehyde for 10 min, washed with PBS, and treated for 10 min each with 10 μg/ml proteinase K in 50 mM Tris-HCl, 5 mM EDTA, pH 8.0 and then 0.25% acetic anhydride in 0.1 M triethanolamine, pH 8.0. The slides were dehydrated by sequential 5-min incubations with 70%, 85%, 95%, and 100% ethanol, followed by xylene. The sections were then incubated for 2 h at 42° C. with 200 μl of prehybridization buffer, which contained 50% formamide, 4×SSC, 5× Denhardt's solution, 500 μg/ml denatured salmon sperm DNA, and 250 μg/ml yeast RNA. For hybridization, the sections were incubated for 24 h at 42° C. with 80 μl of the above buffer containing 1 μg/ml sense or anti-sense DIG-labeled RNA probe. Next, the sections were incubated sequentially as follows: 30 min at 420C with 50% formamide and 1×SSC; 15 min at 37° C. with 0.5×SSC; 15 min at 370C with 3.5×SSC containing 20 μg/ml RNase A; 15 min at room temperature with 3.5×SSC; and 60 min at 60° C. with 0.1×SSC. For detection of the signal, the sections were incubated for 2 h with 1:200 anti-DIG alkaline phosphataseconjugated antibody, washed with PBS, and then developed with alkaline phosphatase substrate (Roche Molecular Biochemicals) for 16 h. The sections were then dehydrated, rehydrated, and counterstained with 3% neutral red for 5 min.

Laser capture microdissection (LCM) and RNA extraction Aortic roots with atherosclerotic lesions were removed from

Apoe−/− mice fed the Western-type diet for 13 weeks. The roots were embedded in OCT compound (VWR Scientific, Bridgeport, N.J.), and frozen immediately on dry ice.

Cryostat sections (6-μm thickness) were mounted on positively charged slides (Color Frost Plus; Fisher Scientific). Lesional macrophages were procured by LCM using a PixCell II LCM System (Arcturus, Mountain View, Calif.) as previously described (51). Briefly, the macrophages of dehydrated sections were stained with CD68 antibody (Serotec, Raleigh, N.C.), and the positively stained areas were selected and affixed to thermoplastic film mounted on optically transparent LCM caps (Arcturus). Total RNA was isolated from the selected cells using a Picopure RNA Isolation Kit (Arcturus) following the manufacturer's instructions, and then treated with DNase I (Ambion, Austin, Tex.). RNA concentrations were determined using a RiboGreen RNA Quantitation Kit (Molecular Probes, Inc., Eugene, Oreg.).

Quantitative RT-PCR

Total RNA from resident peritoneal macrophages or from lesional macrophages, obtained by laser capture microdissection, was reversed transcribed into cDNA synthesis using oligo-dT and Superscript II (Invitrogen). Quantitative PCR for Chop and cyclophilin A (CypA) was conducted using the Taqman PCR reagent (ABI) and the ABI PRISM 7700 sequence detection system as described (51). For Chop, the forward and reverse primers were CCACCACACCTGAAAGCAGAA and AGGTGAAAGGCAGGGACTCA, respectively, and the probe was 6FAM-CTGGTCCACGTGCAGTCATGG-TAMRA. For CypA, the forward and reverse primers were GGCCGATGACGAGCCC and TGTCTTTGGAACTTTGTCTGCAA, respectively, and the probe was TGTCTTTGGAACTTTGTCTGCAA. The PCR conditions for Chop cDNA detection were 950C for 1 min, 580C for 30 s, and 720C for 30 s for 40 cycles. For CypA, the conditions were 950C for 1 min and 600C for 10 s for 40 cycles.

Immunofluorescence Detection of CHOP and CD68 in Atherosclerotic Lesions

The mice were anesthetized, blood was withdrawn by cardiac puncture, and the heart was perfused with PBS and then 4% paraformaldehyde. The heart and proximal aorta were harvested and perfused ex vivo with 4% paraformaldehyde and then stored in the same fixative for 16 h at 40C. The specimens were then transferred to 30% sucrose in PBS for 48h at 40C. In preparation for sectioning, the hearts were embedded in OCT compound (Sakura Finetek, Torrance, Calif.) and stored at −700C. Ten-micron thick sections of the proximal aorta were prepared at −200C on a Microm (Walldorf, Germany) microtome cryostat HM 505E, placed on poly-L-lysine-coated glass slides, and briefly air dried. The sections were washed in PBS and permeabilized in PBS containing 0.2% Triton X-100 for 10 min, washed in PBS containing 0.05% Tween-20 for 10 min, and rinsed repeatedly with PBS at room temperature. Blocking was accomplished by incubation with 5% normal goat serum in PBS for 16h at 40C, followed by washing in PBS containing 0.05% Tween-20 for 10 min and rinsing repeatedly with PBS at room temperature.

For interaction with the primary antibodies, the sections were incubated with 2.5% goat serum containing 1 μg/ml of rabbit polyclonal anti-CHOP C-terminal peptide IgG (R-20 from Santa Cruz Biotechnology) or 1:500 rat monoclonal anti-CD68 supernate (FA11 from Serotec) for 1 h at room temperature. In control experiments, the anti-CHOP IgG was preabsorbed with a 5-fold mass excess of the C-terminal peptide before addition to the slides. After the sections were washed in PBS containing Tween-20 and then PBS, the bound primary antibody was visualized by using 7.5 μg/ml Cy3-conjugated goat anti-rabbit IgG or 3 μg/ml Cy3-conjugated goat anti-rat IgG (Jackson ImmunoResearch Laboratories, West Grove, Pa.). The sections were washed with PBS and, for the CHOP experiment, the DNA was stained with the karyophilic dye Hoechst 33258 (140 ng/ml) for 1 min at room temperature. After extensive washing in PBS, the slides were mounted with a coverslip and viewed with an Olympus IX 70 inverted fluorescence microscope equipped with a CoolSNAP CCD camera.

Filipin Staining of Aortic Sections

Frozen sections of proximal aorta were washed in PBS and then fixed with fresh 3% formaldehyde for 1 h at room temperature. The fixed sections were washed with PBS for 10 min to quench the formaldehyde and then incubated with 0.05 mg/ml filipin solution for 2 h at room temperature. After washing in PBS, the sections were viewed by fluorescence microscopy using an Olympus IX 70 inverted microscope equipped with a UV filter set (340-380-nm excitation, 400-nm dichroic, and 430-nm long pass filters).

Statistics

Results are given as means±S.E.M; n=3 unless otherwise noted.

Results

Cholesterol Enrichment of the Plasma Membrane is Insufficient to Induce Apoptosis in Free Cholesterol-loaded Macrophages.

At high, micromolar concentrations, the amphipathic amine U18666A inhibits all cholesterol trafficking from late endosomes in macrophages that have ingested lipoprotein particles (24). However, when used at lower, nanomolar concentrations, U18666A selectively interferes with cholesterol trafficking to the endoplasmic reticulum (ER) without substantively affecting the transfer of cholesterol to the plasma membrane (24). This is shown here by the observations that low dose U18666A reduced re-esterification of ingested cholesterol, an event that takes place in the ER, by 90% (FIG. 15A) but had no effect on the accrual of cholesterol in the plasma membrane. The latter was assayed by both accessibility of plasma membrane cholesterol to exogenous cholesterol oxidase in lipoprotein-loaded, gluteraldehyde-fixed cells (FIG. 15B) and by extractability of cholesterol by methyl-β-cyclodextrin at 4° C. (data not shown). U18666A has no direct inhibitory effect on the ACAT enzyme itself (24). Low dose U18666A thus provides a useful tool to selectively examine the role of free cholesterol trafficking to the ER in macrophage cell death.

High concentrations of U18666A that inhibit all cholesterol trafficking were previously noted to block macrophage death induced by cholesterol loading (13). Therefore we tested whether lower doses of U18666A, which selectively impair trafficking to the ER membrane, are also able to protect macrophages. Free cholesterol accumulation (loading) was effected by incubating mouse peritoneal macrophages with acetyl-LDL in the presence of the acyl-CoA:cholesterol acyltransferase (ACAT) inhibitor 58035; the latter inhibits cholesterol re-esterification and thus prevents the conversion of the ingested cholesterol into an inert storage form as cholesteryl ester (2,25). FC loading was toxic to macrophages and led to their apoptosis (FIG. 15C), as predicted from our previous work using TUNEL and caspase assays (11). However, even at low doses that selectively impair cholesterol trafficking to the ER, U18666A protected macrophages from the lethal effect of cholesterol loading (FIG. 15C). U18666A failed to protect macrophages from apoptosis induced by the phosphatase inhibitor staurosporine, attesting to the specificity of its protective effect (data not shown). Moreover, androstenediol, a structurally related homologue of U18666A that does not block cholesterol trafficking to the ER in mouse peritoneal macrophages (26), did not block FC-induced cell death, even at concentrations higher than 1 μM (FIG. 15D).

NPC1 is a protein known to play an important role in intracellular cholesterol trafficking (27). We have noted that peritoneal macrophages derived from Npc1+/−mice have a cholesterol trafficking defect that closely mimics that of cells treated with low dose U18666A (28). Therefore, we compared FC-induced death in macrophages from Npc1+/+ and Npc1+/−mice. Like macrophages treated with low dose U18666A, Npc1+/−macrophages were also markedly protected from FC-induced death but not from other inducers of macrophage apoptosis (FIG. 15E).

These data are consistent with a role for cholesterol trafficking to the ER rather than to the plasma membrane in FC-induced macrophage apoptosis. To further test this model, we directly loaded the plasma membrane of macrophages with excess FC by incubating the cells with cholesterol-saturated cyclodextrin (CD-cholesterol, a cyclic oligosaccharide that solubilizes FC) in the presence of the ACAT inhibitor 58035. CD-cholesterol, which bypasses the endocytic route used by lipoproteins, transfers FC directly to the plasma membrane but inefficiently to the ER (29-31). Indeed, CD-cholesterol increased plasma membrane FC to levels found in macrophages incubated with acetyl-LDL plus 58035 (FIG. 15F). However, the cells incubated with CD-cholesterol displayed much less death than those loaded through the endocytic pathway by exposure to lipoproteins (FIG. 15G). These data indicate that the accrual of excess FC in the plasma membrane is insufficient to induce apoptosis in FC-loaded macrophages, further highlighting the potential importance of the ER.

Cholesterol Loading of Macrophages Activates the Unfolded Protein Response (UPR).

As demonstrated above, the protective effects of low dose U18666A and the Npc1+/−mutation are associated with inhibition of cholesterol trafficking to the ER. Because ER membranes are normally cholesterol-poor (15), we sought to determine if excess trafficking of FC to the ER would perturb organelle function. To this end, we monitored activity in the unfolded protein response (UPR) in FC-loaded macrophages under conditions permissive or non-permissive for cholesterol trafficking to ER membranes. Signaling in the UPR is initiated by ER-localized transmembrane protein kinases that become phosphorylated under conditions of impaired organelle function (16). These kinases, in turn, activate downstream transcription factors that promote a gene expression program consisting of many downstream targets involved in restoring ER function and, when stress is severe, in the induction of programmed cell death (16).

The downstream transcription factor CHOP (also known as GADD153) is a marker for UPR activation (32). We found that CHOP induction in FC-loaded macrophages was comparable in magnitude to that elicited by tunicamycin, an agent that activates the UPR by blocking N-linked glycosylation, or by the calcium ionophore A23187, which causes calcium depletion from the ER lumen (FIG. 16A). Blocking cholesterol traffic to ER membranes by low dose U18666A attenuated CHOP induction in FC-loaded macrophages (FIG. 16A, top panel, lane 5). Androstenediol, a homologue of U18666A that does not block cholesterol trafficking to the ER (26), did not block FC-induced CHOP expression, even at concentrations higher than 1 μM (data not shown). Importantly U18666A did not block induction of CHOP by A23187 (FIG. 16A, top panel, lane 6), indicating that U18666A is neither a direct inhibitor of CHOP expression nor a general inhibitor of the UPR. As a control for the effects of U18666A, androstenediol, the homologue of U18666A that does not block cholesterol trafficking (above), did not block FC-induced CHOP induction, even at concentrations higher than 1 μM (FIG. 16A, bottom panel).

CHOP induction in the UPR depends on activation of the ER-resident protein kinase PERK, which induces the upstream transcription factor ATF-4 (33). PERK is specifically activated by impaired protein folding in the ER lumen, a common outcome of many perturbations of organelle function (34). PERK activation entails transautophosphorylation, which is easily detected as a shift in the protein's mobility on immunoblot (34,35). Most of the PERK extracted from cultured macrophages migrated as a faster-mobility, inactive, dephosphorylated form (FIG. 16B, lane 1). Cholesterol loading led to a marked increase in the slower-mobility, activated, phosphorylated PERK, whereas low-dose U18666A, which blocks FC trafficking to the ER, blocked PERK activation in FC-loaded macrophages (FIG. 16B, lanes 4 and 5). As predicted, the changes in PERK activation were reflected in the subsequent expression of its downstream effectors ATF4 and CHOP, detected by immunoblotting nuclear extracts from the same cells (FIGS. 16B, 16C). Note that exposure to the ACAT inhibitor 58035 alone or incubation with acetyl-LDL alone was sufficient to partially activate PERK (FIG. 16B, lanes 2 & 3). These observations suggest that ER function may be perturbed by even small increases in cellular FC.

To confirm the link between FC loading and the UPR, we examined the activity of a second, independent signaling pathway active in the UPR. Like PERK, IRE1 is an ER-localized transmembrane protein kinase whose activity is controlled by transautophosphorylation (16), as reflected by a shift in mobility on immunoblot. In mammalian cells, ER stress-mediated IRE1 activation is absolutely required for expression of the active form of XBP-1, a transcription factor which serves as IREl's effector. Unlike CHOP, whose expression can be induced by other stress signals (36), XBP-1 is highly specific to the UPR (37, 38). FC loading activated IRE1α(the isoform of IRE1 expressed in macrophages) and promoted XBP-1 expression (FIG. 16D, lane 4). Low-dose U18666A markedly attenuated both events (FIG. 16D, lane 5).

To further substantiate the importance of intracellular cholesterol trafficking in UPR activation using a molecular genetic approach, we compared the induction of ATF-4, CHOP, and XBP-1 in Npc1+/+ and Npc1+/−macrophages. As noted above, the Npc1+/−mutation blocks cholesterol trafficking to the ER (28) and blocks FC-induced apoptosis (FIG. 15E). As shown in FIG. 16E, the expression of all three UPR proteins was markedly decreased in FC-loaded Npc1+/−vs. Npc1+/+macrophages. As a control, we show that induction of these three UPR proteins by A23187 is not blocked by the Npc1+/−mutation. In summary, the experiments in FIGS. 16A-16E establish that FC loading of macrophages activates the UPR and that manipulations that block cholesterol trafficking to ER membranes prevent this activation.

As mentioned above, FC-induced macrophage death is thought to be an important event in the progression of atherosclerotic lesions. To determine if advanced atherosclerosis is associated with activation of the UPR, we studied the atherosclerotic lesions of fat-fed Apoe−/− mice. CHOP expression, assessed by in-situ histohybridization and immunohistochemistry, was markedly elevated in the proximal aortic lesions from Apoe−/− mice (FIG. 17A). Note that the anti-sense signal in the histohybridization (blue) appeared scattered throughout the intima, being concentrated in areas that contain cells (marked by the reddish brown-stained nuclei). Staining was absent from lesions hybridized with a sense probe. Chop expression was also assessed using quantitative RT-PCR on RNA obtained from laser-capture microdissection of macrophages from advanced atherosclerotic lesions and from peritoneal macrophages from the same mice. Lesional macrophages expressed a much higher level of Chop mRNA (standardized with CypA mRNA) than peritoneal macrophages (FIG. 17B). CHOP immunostaining was widespread throughout the intima and correlated with Hoechst 3358-stained nuclei (FIG. 17C), consistent with CHOP's known localization to the nucleus (22). Pre-absorption of the antiserum with its cognate peptide abolished the signal (FIG. 17C, third panel), and lesions stained with secondary antibody alone likewise showed no signal (not shown). Moreover, as shown in FIG. 17D, many of the CHOP-expressing cells in atherosclerotic lesions are in macrophage-rich (CD68-positive) and FC-rich (filipin-positive) areas of lesions.

These observations demonstrate that CHOP is expressed in vivo under conditions of perturbed cellular cholesterol metabolism.

FC Loading of Macrophages Leads to Early Depletion of ER Calcium Stores.

The increased FC content of ER membranes, inferred from the trafficking experiments, could perturb ER function at multiple levels. One possibility includes disturbance of calcium homeostasis, which depends on a Ca²⁺ pump and Ca²⁺ release channels embedded in the ER membrane, because depletion of ER calcium stores is a potent inducer of the UPR (39). For example, both thapsigargin, an inhibitor of the ER Ca²⁺ pump, and A23187, a calcium ionophore, lead to dysfunction of calcium-dependent ER chaperones and induce the UPR (39).

We compared ER calcium pools in macrophages incubated for 2.5 h with acetyl-LDL plus 58035 (FC loading conditions) with those incubated with acetyl-LDL and 58035 in the presence of 70 nM U18666A (which inhibits trafficking to the ER). Cells exposed to U18666A alone or culture media with no additives served as further control groups. After perturbation of cellular cholesterol metabolism, the cells were loaded with the fluorescent calcium indicator Fura-2, switched to calcium-free medium, and exposed to 1 μM thapsigargin. Thapsigargin treatment results in emptying of ER calcium stores into the cytosol, which was quantified by fluorescence microscopy, providing a measure of releasable ER calcium stores at the time of thapsigargin addition.

Representative tracings of the Fura-2 fluorescence ratio (340:380 nm) for individual macrophages in each experimental group are displayed in FIG. 18A. The untreated macrophage (blue line) displayed a sharp rise in cytosolic calcium soon after the addition of thapsigargin, indicating an abundance of releasable, ER-stored calcium.

Similar results were found with the macrophage incubated with U18666A alone (black line). By contrast, the FC-loaded macrophage (green line) had a markedly blunted response, indicating depleted ER calcium stores.

Macrophages loaded with FC for longer periods of time showed no increase in cytosolic calcium following thapsigargin treatment (data not displayed), indicating severely depleted ER calcium stores. Importantly, the macrophage loaded with FC in the presence of 70 nM U18666A (red line) showed a similar rise in cytosolic calcium as the control macrophages. Analysis of the basal-to-peak increment in fluorescent ratio of multiple individual cells in each group clearly demonstrates that FC-loading is associated with a substantially smaller thapsigargin-induced rise in cytosolic calcium, an effect which is completely prevented by treatment with 70 nM U18666A (FIG. 18B). Because depletion of ER calcium stores can activate capacitive calcium entry, we determined the effect of blockers of this process on FC-induced apoptosis and CHOP induction. While SKF96365 could not be used due to non-specific effects on cholesterol metabolism, we showed that neither 0.1 mM NiCl2 nor 10 μM cadmium succinate affected apoptosis or CHOP induction in FC-loaded macrophages. From these data we conclude that depletion of ER calcium stores is an early event in FC loading that is dependent on intracellular cholesterol trafficking to the ER. Given that ER calcium depletion is a known UPR inducer, this event likely contributes to FC-induced UPR activation.

UPR Signaling Modulates FC-Induced Apoptosis in Macrophages.

UPR signaling plays an important role in modulating the sensitivity of cells to death induced by perturbations that cause ER stress. Cells lacking PERK are markedly hypersensitive to agents that cause ER stress (33), and animals and human with PERK mutations undergo rapid loss of certain populations of secretory cells that are exposed to physiologically high levels of ER stress (40,41). In contrast, CHOP is an effector of cell death induced by ER stress, as Chop−/− cells are partially protected from death induced by agents and conditions that impair ER function (22, 23, 42). Therefore, the signal transduction pathways downstream of PERK have protective effects that predominate, but also include a CHOP-dependent death-promoting arm that can be selectively inactivated by CHOP deletion.

If ER dysfunction plays an important role in the death of FC-loaded macrophages, PERK deletion should increase cell death, whereas CHOP deletion should protect against death. To test this hypothesis, we compared the response to FC loading of macrophages procured from wild-type mice, Chop−/−, and Perk−/− mice. Cell death, measured by annexin V and propidium iodide staining, was dramatically increased in FC-loaded Perk−/− macrophages compared with wild-type macrophages (FIG. 19A, 19B). Importantly, low dose U18666A markedly protected the Perk−/− cells, implicating the importance of FC trafficking to the ER in the death of Perk−/− cells (FIGS. 19A, 19B).

By contrast, the majority of Chop−/− macrophages were protected from cell death induced by FC loading, and this protection, relative to the wild-type cells, was noted at both early (16 hours) and late time points (27 hours) (FIGS. 20A, 20B). The percentage of macrophages with activated caspase-3, which is critical for FC-induced apoptosis (11), was also markedly decreased in the Chop−/− macrophages (FIG. 20C). Neither the Perk nor the Chop mutations significantly affected the mass of lipoprotein-FC accumulated by the macrophages (data not shown). These data establish a functional role for the UPR in FC-induced apoptosis in macrophages. Moreover, consistent with the notion that the Chop−/− mutation blocks FC-induced macrophage death via a distal UPR pathway, and not by inhibiting upstream events, induction of XBP-1 and ATF-4, as well as depletion of ER calcium stores, were similar in FC-loaded Chop+/+ and Chop−/− macrophages (FIGS. 20D, 20E).

Discussion

Cell possess multiple mechanisms to prevent the accumulation of excess FC, including activation of ACAT-mediated cholesterol esterification, induction of cellular cholesterol efflux, and repression of the LDL receptor and cholesterol biosynthetic enzymes (1, 2). When one or more of these mechanisms fail, as appears to happen in macrophages in advanced atherosclerotic lesions, cell death ensues. Most interestingly, the intracellular accumulation of large amounts of cholesteryl ester, as occurs in the macrophage foam cells of early atherosclerotic lesions, does not induce cell death. The fact that cholesteryl esters are stored in relatively inert intracellular lipid vesicles, whereas free cholesterol is integrated into the lipid bilayers of cellular membranes, has been seized on as a possible clue to the toxic effects of the latter. The purpose of this study was to provide insight into the perturbations effected by FC loading of macrophages.

We reasoned that elucidating the site of FC accumulation may give a clue to mechanism of FC-induced toxicity. Although the direct measurement of FC mass in biological membranes is very difficult, plasma membrane FC content can be estimated by both the cholesterol oxidase method (43) and by availability to cyclodextrin-mediated efflux (44), and ER cholesterol can be assessed by esterification by the ER enzyme ACAT (45). Using these methods, earlier work had focused attention on the role of FC accumulation in the plasma membrane in FC-induced macrophage death based on the protective effect of inhibitors of FC trafficking from the endosomes, such as U18886A (10, 13). While doses that inhibit trafficking to the plasma membrane clearly protect macrophages from FC-induced death, the fact that these doses also inhibit trafficking to other cellular sites such as the ER and mitochondria was not addressed. In this study, we showed that both low-dose U18666A and a genetic manipulation that selectively blocks trafficking of FC to the ER but not to the plasma membrane block macrophage death. Moreover, directly loading the plasma membrane with FC had no effect on macrophage viability. Thus, FC loading of the plasma membrane to levels attained in this model system is not sufficient to promote apoptosis. FC loading of mitochondria is also unlikely to contribute to macrophage death, because incubation of mitochondria in vitro with excess cholesterol actually stabilizes mitochondrial function (46; unpublished data from our laboratory). The ER membrane, in contrast to the plasma membrane, is cholesterol-poor and fluid (15) and thus predicted to be especially sensitive to the toxic effects of FC enrichment. Indeed, we find that an ER-based signal transduction pathway is induced by FC loading. Together, these findings point to the ER and not the plasma membrane as a major source of FC toxicity.

How might FC trafficking to ER membranes lead to ER stress? Many of the biochemical activities essential to client protein folding and processing rely on ER membrane proteins, and these may be perturbed by increasing the FC:phospholipid ratio of the normally fluid ER membrane. In this report, we have shown that a critical ER membrane protein-dependent function, namely maintenance of ER calcium homeostasis, is perturbed early in the course of FC loading. The fact that this effect required cholesterol trafficking to the ER membrane and that depletion of ER calcium stores by other agents induces the UPR suggests that this event is at least part of the mechanism linking FC loading and UPR induction. It is also possible that FC loading of the ER membrane perturbs other ER membrane-dependent activities, either as a primary event or secondary to the early depletion of ER calcium stores. Of interest, modest levels of FC loading are sufficient to activate the PERK kinase in macrophages (FIG. 16B, lanes 2 and 3), suggesting that the ER in these cells may normally function close to its threshold for FC tolerance.

An important finding in this report is that UPR signaling has a profound effect on the response of macrophages to FC loading. Our observations that FC loading activates PERK and that Perk−/− macrophages are markedly hypersensitive to FC loading indicate that the ability to resist ER stress can play a limiting role in the survival of FC-loaded macrophages. ER stress-induced cell death proceeds by several known pathways, including CHOP-induced decreases in bcl-2 and glutathione and increases in reactive oxygen species (42). While ˜30% of FC-induced macrophage death still occurs in the presence of the Chop−/− mutation, suggesting a relatively minor role for a non-CHOP pathway, our observation that the majority of Chop−/− macrophages are resistant to FC-induced cell death and caspase-3 activation argues that a pro-apoptotic pathway activated specifically by the CHOP arm of the UPR plays an important role in promoting apoptosis under these circumstances. In this regard, the expression of CHOP in macrophage and FC-rich areas of atherosclerotic lesions, the tendency of advanced lesional macrophages to accumulate FC, and the association of macrophage death with plaque disruption suggest that ER stress in lesional macrophages may be an important cellular process in the progression of atherosclerosis.

References for Third Series of Experiments

1. Brown, M. S. & Goldstein, J. L. The SREBP pathway: regulation of cholesterol metabolism by proteolysis of a membrane-bound transcription factor. Cell 89:331-340 (1997).

2. Tabas,I. Consequences of cellular cholesterol accumulation: basic concepts and physiological implications. J. Clin. Invest. 110:905-911 (2002).

3. Shio,H., Haley,N.J. & Fowler,S. Characterization of lipid-laden aortic cells from cholesterol-fed rabbits. III. Intracellular localization of cholesterol and cholesteryl ester. Lab. Invest. 41:160-167 (1979).

4. Rapp,J. H., Connor,W. E., Lin,D. S., Inahara,T. & Porter,J. M. Lipids of human atherosclerotic plaques and xanthomas: clues to the mechanism of plaque progression. J. Lipid Res. 24:1329-1335 (1983).

5. Small,D. M., Bond,M. G., Waug,D. , Prack,M. & Sawyer,J. K. Physicochemical and histological changes in the arterial wall of nonhuman primates during progression and regression of atherosclerosis. J. Clin. Invest. 73:1590-1605 (1984).

6. Kruth, H. S. & Fry, D. L. Histochemical detection and differentiation of free and esterified cholesterol in swine atherosclerosis using filipin. Exp. Mol. Pathol. 40:288-294 (1984).

7. Libby,P. & Clinton,S. K. The role of macrophages in atherogenesis. Curr. Opin. Lipidol. 4:355-363 (1993).

8. Ball, R. Y. et al. Evidence that the death of macrophage foam cells contributes to the lipid core of atheroma. Atherosclerosis 114:45-54 (1995).

9. Fazio,S. et al. Increased atherosclerosis in LDL receptor-null mice lacking ACAT1 in macrophages. J. Clin. Invest. 107:163-171 (2001).

10. Kellner-Weibel, G. et al. Effects of intracellular free cholesterol accumulation on macrophage viability: a model for foam cell death. Arterioscler. Thromb. Vasc. Biol. 18:423-431 (1998).

11. Yao, P. M. & Tabas, I. Free cholesterol loading of macrophages induces apoptosis involving the fas pathway. J. Biol. Chem. 275:23807-23813 (2000).

12. Yao, P. M. & Tabas, I. Free cholesterol loading of macrophages is associated with widespread mitochondrial dysfunction and activation of the mitochondrial apoptosis pathway. J. Biol. Chem. 276:42468-42476 (2001).

13. Kellner-Weibel, G., Geng, Y. J. & Rothblat, G. H. Cytotoxic cholesterol is generated by the hydrolysis of cytoplasmic cholesteryl ester and transported to the plasma membrane. Atherosclerosis 146:309-319 (1999).

14. Yeagle, P. L. Modulation of membrane function by cholesterol. Biochimie 73:1303-1310 (1991).

15. Bretscher, M. S. & Munro, S. Cholesterol and the Golgi apparatus. Science 261:1280-1281 (1993).

16. Patil, C. & Walter, P. Intracellular signaling from the endoplasmic reticulum to the nucleus: the unfolded protein response in yeast and mammals. Curr. Opin. Cell Biol. 13:349-355 (2001).

17. Travers, K. J. et al. Functional and genomic analyses reveal an essential coordination between the unfolded protein response and ER-associated degradation. Cell 101:249-258 (2000).

18. Zhang, D. et al. Macrophages deficient in CTP: Phosphocholine cytidylyltransferasealpha are viable under normal culture conditions but are highly susceptible to free cholesterol-induced death. Molecular genetic evidence that the induction of phosphatidylcholine biosynthesis in free cholesterol-loaded macrophages is an adaptive response. J. Biol. Chem. 275:35368-35376 (2000).

19. Nakagawa, T. et al. Caspase-12 mediates endoplasmic-reticulum-specific apoptosis and cytotoxicity by amyloid-b. Nature 403:98-103 (2000).

20. Urano, F. et al. Coupling of stress in the ER to activation of JNK protein kinases by transmembrane protein kinase IRE1. Science 287:664-666 (2000).

21. Nishitoh, H. et al. ASK1 is essential for endoplasmic reticulum stress-induced neuronal cell death triggered by expanded polyglutamine repeats. Genes Dev. 16:1345-1355 (2002).

22. Zinszner,H. et al. CHOP is implicated in programmed cell death in response to impaired function of the endoplasmic reticulum. Genes Dev. 12:982-995 (1998).

23. Oyadomari,S. et al. Targeted disruption of the Chop gene delays endoplasmic reticulum stress-mediated diabetes. J. Clin. Invest. 109:525-532 (2002).

24. Underwood,K. W., Andemariam,B., McWilliams,G. L. & Liscum,L. Quantitative analysis of hydrophobic amine inhibition of intracellular cholesterol transport. J. Lipid Res. 37:1556-1568 (1996).

25. Shiratori, Y., Okwu, A. K. & Tabas, I. Free cholesterol loading of macrophages stimulates phosphatidylcholine biosynthesis and up-regulation of CTP:phosphocholine cytidylyltransferase. J. Biol. Chem. 269:11337-11348 (1994).

26. Aikawa, K., Furuchi, T., Fujimoto, Y., Arai, H. & Inoue, K. Structure-specific inhibition of lysosomal cholesterol transport in macrophages by various steroids. Biochim. Biophys. Acta 1213:127-134 (1994).

27. Liscum, L. & Klansek, J. J. Niemann-Pick disease type C. Curr. Opin. Lipidol. 9:131-135 (1998).

28. Feng, B. & Tabas, I. ABCA1-mediated cholesterol efflux is defective in free cholesterol-loaded macrophages. Mechanism involves enhanced ABCA1 degradation in a process requiring full NPC1 activity. J. Biol. Chem. 277:43271-43280 (2002).

29. Yancey, P. G. et al. Cellular cholesterol efflux mediated by cyclodextrins. Demonstration Of kinetic pools and mechanism of efflux. J. Biol. Chem. 271:16026-16034 (1996).

30. Christian, A. E., Haynes, M. P., Phillips, M. C. & Rothblat, G. H. Use of cyclodextrins for manipulating cellular cholesterol content. J. Lipid Res. 38:2264-2272 (1997).

31. Khan, N. et al. Plasma membrane cholesterol: A possible barrier to intracellular oxygen in normal and mutant CHO cells defective in cholesterol metabolism. Biochemistry 42:23-29 (2003).

32. Wang, X. Z. & Ron, D. Stress-induced phosphorylation and activation of the transcription-factor CHOP (GADD153) by p38 MAP Kinase. Science 272:1347-1349 (1996).

33. Harding, H. P., Zhang, Y., Bertolotti, A., Zeng, H. & Ron, D. Perk is essential for translational regulation and cell survival during the unfolded protein response. Mol. Cell 5:897-904 (2000).

34. Bertolotti, A., Zhang, Y., Hendershot, L. M., Harding, H. P. & Ron, D. Dynamic interaction of BiP and ER stress transducers in the unfolded-protein response. Nat. Cell Biol. 2:326-332 (2000).

35. Harding, H. P., Zhang, Y. & Ron, D. Protein translation and folding are coupled by an endoplasmic-reticulum-resident kinase. Nature 397:271-274 (1999).

36. Harding, H. P. et al. Regulated translation initiation controls stress-induced gene expression in mammalian cells. Mol. Cell 6:1099-1108 (2000).

37. Yoshida, H., Matsui, T., Yamamoto, A., Okada, T. & Mori, K. XBP1 mRNA is induced by ATF6 and spliced by IRE1 in response to ER stress to produce a highly active transcription factor. Cell 107:881-891 (2001).

38. Calfon, M. et al. IRE1 couples endoplasmic reticulum load to secretory capacity by processing the XBP-1 mRNA. Nature 415:92-96 (2002).

39. Treiman,M. Regulation of the endoplasmic reticulum calcium storage during the unfolded protein response—significance in tissue ischemia? Trends Cardiovasc. Med. 12: 57-62 (2002).

40. Harding, H. P. et al. Diabetes mellitus and exocrine pancreatic dysfunction in Perk−/− mice reveals a role for translational control in secretory cell survival. Mol. Cell 7:1153-1163 (2001).

41. Zhang, P. et al. The PERK eukaryotic initiation factor 2 alpha kinase is required for the development of the skeletal system, postnatal growth, and the function and viability of the pancreas. Mol. Cell Biol. 22:3864-3874 (2002).

42. McCullough, K. D., Martindale, J. L., Klotz, L. O., Aw, T. Y. & Holbrook, N.J. Gaddl53 sensitizes cells to endoplasmic reticulum stress by down-regulating Bcl2 and perturbing the cellular redox state. Mol. Cell Biol. 21:1249-1259 (2001).

43. Lange, Y. Tracking cell cholesterol with cholesterol oxidase. J. Lipid Res. 33:315-321 (1992).

44. Lange, Y., Ye, J., Rigney, M. & Steck, T. L. Cholesterol movement in Niemann-Pick Type C cells and in cells treated with amphiphiles. J. Biol. Chem. 275:17468-17475 (2000).

45. Lange, Y. & Steck, T. L. Quantitation of the pool of cholesterol associated with acyl-CoA:cholesterol acyltransferase in human fibroblasts. J. Biol. Chem. 272:13103-13108 (1997).

46. Graham, J. M. & Green, C. The properties of mitochondria enriched in vitro with cholesterol. Eur. J. Biochem. 12:58-66 (1970).

47. Ross, A. C., Go, K. J., Heider, J. G. & Rothblat, G. H. Selective inhibition of acyl coenzyme A:cholesterol acyltransferase by compound 58-035. J. Biol. Chem. 259:815-819 (1984).

48. Basu, S. K., Goldstein, J. L., Anderson, R. G. W. & Brown, M. S. Degradation of cationized low density lipoprotein and regulation of cholesterol metabolism in homozygous familial hypercholesterolemia fibroblasts. Proc. Natl. Acad. Sci. USA 73:3178-3182 (1976).

49. Tabas, I., Boykow, G. & Tall, A. Rabbit liver microsomal ACAT: Smooth ER enzyme associated with a lipid ACAT inhibitor. Arteriosclerosis 8:559A (1988).

50. Lowry, O. H., Rosenbrough, N.J., Farr, A.L. & Randall, R. J. Protein measurement with the folin phenol reagent. J. Biol. Chem. 193:265-275 (1951).

51. Trogan, E. et al. Laser capture microdissection analysis of gene expression in macrophages from atherosclerotic lesions of apolipoprotein E-deficient mice. Proc. Natl. Acad. Sci. U.S.A. 99:2234-2239 (2002).

Fourth Series of Experiments

Synopsis

Macrophage death in advanced atherosclerotic lesions leads to lesional necrosis and likely promotes plaque instability, a precursor of acute vascular events. Macrophages in advanced lesions accumulate large amounts of unesterified cholesterol, which is a potent inducer of macrophage apoptosis. We have shown recently that induction of apoptosis in cultured macrophages requires cholesterol trafficking to the endoplasmic reticulum (ER). Moreover, macrophages from mice with a heterozygous mutation in the cholesterol-trafficking protein Npcl have a selective defect in cholesterol trafficking to the ER and are protected from cholesterol-induced apoptosis. The goal of the present study was to test the importance of intracellular cholesterol trafficking in atherosclerotic lesional macrophage death by comparing lesion morphology in Npc1+/+;Apoe−/− and Npc1+/−;Apoe−/− mice. Although advanced lesions in Npc1+/+;Apoe−/− mice had extensive acellular areas that were rich in unesterified cholesterol and macrophage debris, the lesions of Npc1+/−;Apoe−/− mice were substantially more cellular and less necrotic. Moreover, compared with Npc1+/−;Apoe−/− lesions, Npc1+/+;Apoe−/− lesions had a greater number of large, TUNEL (terminal deoxynucleotidyltransferase-mediated dUTP nick end labeling)-positive areas surrounding necrotic areas, indicative of macrophage apoptosis. These differences were observed despite similar total lesion area and similar plasma lipid levels in the two groups of mice. These data provide in vivo evidence that intact intracellular cholesterol trafficking is important for macrophage apoptosis in advanced atherosclerotic lesions and that the ER-based model of cholesterol-induced cytotoxicity is physiologically relevant. Moreover, by showing that lesional necrosis can be diminished by a subtle defect in intracellular trafficking, these findings suggest therapeutic strategies to stabilize atherosclerotic plaques.

Introduction

Macrophages are major cellular components of developing atherosclerotic lesions, and they play important roles in atherogenesis (1,2). Interestingly, lesional macrophages undergo cell death (3,4), although little is known about the consequences or causes of this event. Recent data with antibodies against cell-specific proteins support the idea that the macrophage is the main cell type that dies in the vicinity of lesional necrotic areas (5,6). These necrotic areas are often found near sites of plaque rupture, which is directly linked to acute thrombosis, vascular occlusion, and tissue infarction (7). Indeed, the presence of apoptotic macrophages is associated specifically with ruptured atherosclerotic plaques in human coronary artery lesions (8). The mechanistic link between macrophage death and unstable plaques may be related to plaque-destabilizing enzymes, inflammatory mediators, and procoagulant and thrombogenic molecules released by these dying cells (2).

The causes of macrophage death in advanced atherosclerosis are not known. An important cytotoxic condition that deserves attention is excess cellular free cholesterol (FC) (9). FC accumulation in lesional foam cells has been well documented (10-14), and studies with cultured macrophages have shown that excess cellular FC is a potent inducer of cell death (15-18). Moreover, acceleration of FC accumulation in lesional macrophages in mice by targeted disruption of Soatl leads to increased lesional macrophage apoptosis (19). FC-induced death in cultured macrophages, like lesional macrophage death in vivo, has both apoptotic and necrotic features (3,4), and it is likely that at least a portion of the necrosis is secondary to defective phagocytic clearance of apoptotic macrophages (postapoptotic necrosis) (20,21).

Recent work in our laboratory has revealed important mechanistic aspects of FC-induced macrophage apoptosis. By using cultured macrophages incubated with a source of lipoprotein cholesterol (acetylated low-density lipoprotein) and an acyl-CoA:cholesterol acyltransferase inhibitor to block cholesterol esterification, we have shown that apoptotic death of FC-loaded cultured macrophages is caused by both activation of Fas ligand and release of cytochrome c from mitochondria (17,18). Most interestingly, FC-induced apoptosis in macrophages is entirely dependent on cholesterol trafficking to the endoplasmic reticulum (ER) (22). We have shown that FC loading of cultured macrophages activates the ER stress pathway known as the unfolded protein response (UPR) and that the branch of the UPR involving the transcription factor CHOP(C/EBP homologous protein) is necessary for execution of apoptosis in these cells (22). We have shown also that CHOP is expressed in advanced atherosclerotic lesions of Apoe−/− mice (22).

The trafficking of lipoprotein-derived cholesterol from late endosomes to peripheral cellular membranes is dependent on the late endosomal protein Npc1 (23-26). In the course of our mechanistic studies, we discovered that whereas complete deficiency of Npcl blocks cholesterol trafficking to both the plasma membrane and ER, heterozygous deficiency of Npcl selectively blocks cholesterol trafficking to the ER (22). Consistent with the model described above, cultured peritoneal macrophages from Npc1+/−mice are highly resistant to FC-induced activation of the UPR and FC-induced apoptosis (22).

The goal of the present study was to determine the relevance of these concepts in advanced atherosclerotic lesions of the widely studied Apoe−/− mouse model of atherosclerosis. We accomplished this goal by studying plaque morphology and macrophage apoptosis in advanced atherosclerotic lesions of Npc1+/+;Apoe−/− vs. Npc1+/−;Apoe−/− mice. Our data reveal that the Npc1+/−mutation confers substantial resistance to lesional necrosis and lesional macrophage apoptosis. As such, these data provide in vivo support for the ER-based model of FC-induced apoptosis and suggest therapeutic strategies to promote plaque stability.

Materials and Methods

Mice

Npc1+/−BALB/c mice were backcrossed into the C57BL/6 background for four generations and then bred into the Apoe−/− C57BL/6 mouse background for an additional two generations to generate Npc1+/−;Apoe−/− mice. These mice were bred with each other to obtain Npc1+/−;Apoe−/− mice and Npc1+/+;Apoe−/− mice (littermate controls) for the experiments described below. After weaning, the mice were placed on a high-cholesterol diet containing 21% anhydrous milk fat and 0.15% cholesterol (“Western-type” diet; Harlan-Teklad, Indianapolis) and killed at 18 or 25 weeks of age.

Preparation and Staining of Sections of Proximal Aortae

On the day of analysis, food was removed from the cages in the morning, and the mice were fasted for 7 h. The animals were then anesthetized, and blood was withdrawn by cardiac puncture. The heart and vascular system were perfused with PBS in situ, and the heart and proximal aorta were then removed surgically, embedded in OCT (optimum cutting temperature) compound (Sakura Finetek, Torrance, Calif.), snap frozen in an ethanol-dry-ice bath, and stored at −70° C.

Sections of proximal aortae 10 μm thick were cut at −20° C. by using a Microm Microtome Cryostat HM 505 E, placed on poly-L-lysine-coated slides (Fisher Scientific). The sections were then fixed in 10% buffered formalin for 5 min at room temperature and air dried for 10 min. Serial sections were stained with Harris' hematoxylin for nuclei.

Quantification of Atherosclerotic Lesion, Acellular, and Terminal Deoxynucleotidyltransferase-Mediated dUTP Nick End Labeling (TUNEL)-Positive Areas

Intimal lesional area (between the internal elastic lamina to the lumen), acellular areas (negative for hematoxylinpositive nuclei), and TUNEL-positive areas were quantified by blinded observers using a Nikon Labophot 2 microscope equipped with an Sony CCD-Iris/RGB color video camera attached to a computerized imaging system with IMAGE PRO PLUS 3.0 software. For the TUNEL technique, cryostat sections were air dried and subsequently fixed by 4% buffered formalin. We used the stringent TUNEL method described by Kockx et al. (27), which avoids nonspecific labeling because of active RNA synthesis.

Immunohistochemistry

The mice were anesthetized, blood was withdrawn by cardiac puncture, and the heart was perfused with PBS. The heart and proximal aorta were harvested, perfused ex vivo with PBS, embedded in OCT compound, and snap frozen in an ethanol-dry ice bath. For detection of the type A scavenger receptor, the frozen sections of the proximal aorta were washed in tap water and then rinsed in PBS for 5 min at room temperature. The sections were then incubated in PBS containing 2% donkey serum and 50 μg/ml. rat anti-mouse macrophage type A scavenger receptor antibody (2F8; Serotec), or rat IgG2b nonimmune control antibody, for 1 hour at room temperature. After washing in PBS for 5 min, the sections were incubated with the primary antibody in PBS containing 2% donkey serum. Staining was completed by incubating first with biotinylated donkey anti-rat IgG and then with streptavidin-peroxidase (Vectastain Elite ABC kit, Vector Laboratories) and 3,3′-diaminobenzidine. The sections were counterstained with hematoxylin, rinsed, mounted in Permount, and viewed with an Olympus IX 70 inverted microscope using a X20 objective. Macrophages were detected in acetone-fixed frozen sections by using rabbit antimouse macrophage antiserum from Accurate Chemicals (AIA31240); rabbit nonimmune serum served as the control for this procedure.

Filipin Staining of Aortic Sections

Frozen sections of proximal aortae were washed in PBS and then fixed with fresh 3% formaldehyde for 1 h at room temperature. The fixed sections were washed with PBS for 10 min to quench the formaldehyde and then incubated with 0.05 mg/ml filipin solution for 2 h at room temperature. After washing in PBS, the sections were viewed by fluorescence microscopy using an Olympus IX 70 inverted microscope equipped with a UV filter set (340- to 380-nm excitation, 400-nm dichroic, and 430-nm long-pass filters).

Plasma Lipid and Lipoprotein Assays

Total plasma cholesterol and phospholipids were determined by using commercial enzymatic kits (Wako, Neuss, Germany), and plasma lipoproteins were analyzed by fast performance liquid chromatography, as described (28).

Statistical Analysis

For lesional data, which are nonparametric, statistical significance was determined by the Mann-Whitney U test. For the quantitative TUNEL-staining data, the x² test was used.

Results

Evidence of Necrosis, FC Accumulation, and Macrophage Debris in Advanced Atherosclerotic Lesions of Apoe−/− Mice Apoe−/− mice fed a Western-type diet (high cholesterol/saturated fat) develop advanced atherosclerosis (29,30). In preparation for experiments designed to look at the influence of the Npc1+/−mutation on plaque morphology, we first characterized the advanced lesions of Npc1+/+;Apoe−/− mice. As shown in FIG. 21A, raised lesions from the proximal aortae of 25-week-old Apoe−/− mice fed a Western-type diet contained acellular regions situated beneath a layer of endothelial and intimal cells (FIG. 21, arrows). Using a stain for collagen, we focused first on acellular areas that were not simply dense fibrous scars (data not shown). Next, because extracellular FC accumulation is a property of necrotic areas of advanced atherosclerotic lesions in humans (3,31,32), we stained the lesions with filipin, a fluorescent dye that binds FC (31). As demonstrated in FIG. 21B, most of the acellular areas (as well as many of the cellular areas of the intima) bound filipin, whereas neither the adventitia nor the endothelium bound filipin. Moreover, the acellular areas of Apoe−/− lesions stained only weakly with the neutral lipid dye oil red 0 compared with the cholesteryl ester-rich foam cell areas (data not shown); this finding indicates that the acellular areas were richer in FC than in cholesteryl esters. To determine whether these acellular areas might represent sites of macrophage death as suggested by the studies of Mitchinson and colleagues (6), we looked for the presence of macrophage proteins in the absence of intact cells (debris). As shown in FIG. 21C, many of the acellular regions reacted with an antibody against a macrophage protein, the type A scavenger receptor. In contrast, adjacent sections did not react with nonimmune control antibody (FIG. 21D). Similar results were obtained by using an antibody directed against the macrophage protein Mac-3 (data not shown). Thus, advanced lesions of Apoe−/− mice contain acellular areas that stain for both FC and macrophage proteins, suggesting that these areas are necrotic areas containing the debris of macrophages.

Atherosclerotic Lesions of Npc1+/−;Apoe−/− Mice Have Fewer Acellular Areas Than Lesions of Apoe−/− Mice

To assess the effect of the heterozygous Niemann-Pick C mutation on the atherosclerotic lesion morphology, we studied 25-week-old, Western-diet-fed Npc1+/+;Apoe−/− mice and Npc1+/−;Apoe−/− mice. As was the case with peritoneal macrophages from these mice on the Apoe+/+background (22), peritoneal macrophages from Npc1+/−;Apoe−/− mice showed marked resistance to FC-induced apoptosis compared with Npc1+/+;Apoe−/− macrophages (data not shown). Both groups of mice appeared normal, and their weights at the end of the 25-week period were not statistically different. The plasma cholesterol and phospholipid values were also not statistically different in the two groups of mice (FIG. 22A). Similarly, the gel-filtration profiles of the plasma lipoproteins were very similar (FIG. 22B); neither the small increase in the very low-density lipoprotein peak nor the small decrease in the low-density lipoprotein peak in the Npc1+/−;Apoe−/− plasma shown in this figure was a consistent finding in repeat experiments.

An example of a histological section from each type of mouse is shown in FIGS. 23A-23E. FIG. 23A shows extensive acellular areas (asterisks) in the lesion of an Npc1+/+;Apoe−/− mouse. In contrast, the atherosclerotic lesions of the Npc1+/−;Apoe−/− mice contained many more live cells (FIG. 23B), which were identified as macrophages by reactivity with a rabbit anti-mouse macrophage antiserum (FIG. 23C). The acellular areas in FIG. 23A occupy a substantial amount of lesional volume, which can account for the similar overall sizes of the Npc1+/+ and Npc1+/−lesions despite their difference in cellularity. The increased cellularity in the Npc1+/−could be due to less accumulation of total lesional FC rather than altered intracellular location of this FC, as our model suggests. To assess this possibility, lesions from Npc1+/+;Apoe−/− and Npc1+/−;Apoe−/− mice were stained with filipin. As shown in FIGS. 23D and E, the intensity of filipin staining was similar in the two lesions, suggesting that the increase in cellularity in the Npc1+/−;Apoe−/− mice was not simply due to a marked decrease in overall lesional FC accumulation.

Quantitative data for two separate experiments completed =1 year apart are shown in FIGS. 24A-24F. In the first experiment, total lesion area in the proximal aortae of Npc1+/−;Apoe−/− mice was decreased =20% compared with Npc1+/+;Apoe−/− mice (FIG. 24A), but this difference was not statistically significant. The acellular areas of the Npc1+/−;Apoe−/− lesions, however, were decreased by =50% (FIG. 24B). Therefore, when the data were expressed as percent acellular area, there was a substantial and highly statistically significant difference between the two groups of mice (FIG. 24C). The decrease in acellular areas in the lesions of Npc1+/−;Apoe−/− mice was similar in males and females (data not shown). In the repeat experiment, lesion area per se was almost identical between the two groups of mice (FIG. 24D), and acellular areas were decreased by =35% (FIGS. 24E and F), which reached a very high level of statistical difference. Thus, the proximal aortic lesions of 25-week-old Npc1+/−;Apoe−/− mice have less extensive acellular areas than those of Npc1+/+; Apoe−/− mice.

Atherosclerotic Lesions of Npc1+/−; Apoe−/− Mice Have Fewer TUNEL Positive Apoptotic Areas Than Lesions of Npc1+/+;Apoe−/− Mice

To assess apoptosis in these lesions, sections were stained by using a stringent modification of the TUNEL method (27).

Examples of sections from two Npc1+/+;Apoe−/− mice are shown in FIG. 25A. In both of these examples, large TUNEL-positive areas were present; in FIG. 25A (left panel) such an area was located near an area of widespread necrosis. In the sections from two Npc1+/−;Apoe−/− mice (FIG. 25B), TUNEL staining either was not seen or was less extensive. FIGS. 25C and D show the quantification of lesion area and TUNEL data, respectively, for nine Npc1+/+;Apoe−/− mice and seven Npc1+/−; Apoe−/− mice. Fewer of the Npc1+/−;Apoe−/− lesions contained apoptotic, TUNEL-positive areas despite no significant change in lesion size. Thus, lesional macrophage death is decreased by a mutation that confers a specific defect in intracellular cholesterol trafficking to the ER.

Discussion

Few studies have attempted to link genetic alterations in specific macrophage death pathways directly with consequences for atherosclerotic lesion morphology in vivo.

In the current study, this strategy was inspired by two recent findings by our laboratory using cultured peritoneal macrophages: (i) cholesterol trafficking to the ER, by activation of the ER-based UPR, is necessary for FC-induced apoptosis in macrophages (22), and (ii) macrophages with the Npc1+/−mutation have a specific defect in the trafficking of lipoprotein-derived cholesterol to the ER, resulting in abrogation of both FC-induced UPR activation and FC-induced apoptosis (22). Moreover, the UPR-induced transcription factor CHOP(C/EBP homologous protein) is expressed in atherosclerotic lesions of Apoe−/− mice (22). These previous data were conducted with cells in culture, and the physiological relevance of the method used for FC loading (acetylated low-density lipoprotein plus an acyl-CoA:cholesterol acyltransferase inhibitor) was uncertain. Thus, it was critical to seek evidence for or against the FC-ER-based model of macrophage death in lesional macrophage apoptosis in vivo. The finding that lesional necrosis and macrophage apoptosis were substantially less in the lesions of a mouse with a subtle defect in cholesterol trafficking to the ER provides strong support for this model.

What is the physiological significance of increased macrophage cellularity and decreased apoptosis in Npc1+/−;Apoe−/− lesions? Although it is theoretically possible that increased numbers of macrophages could contribute to lesion pathology (33), overall lesion size was not increased in the Npc1+/−; Apoe−/− mouse model. Rather, the increased cellularity was associated with less lesional necrosis. There is evidence that macrophage death contributes to the development of lesional necrotic areas, which have been called “a graveyard of dead macrophages” (34). For example, necrotic areas of human lesions have been shown to contain macrophage proteins (5,6), consistent with our data (FIG. 21C), and dying macrophages are often found in the vicinity of lesional necrosis, (2,3,32). Because atherosclerotic lesions rich in necrotic areas are susceptible to plaque rupture, acute thrombosis, and acute vascular occlusion (7), the increase in cellularity may be a sign of plaque stabilization. Future studies will be needed to determine whether Npc1+/−;Apoe−/− lesions are indeed less susceptible to plaque rupture per se.

In summary, we have demonstrated that partial dysfunction of a specific protein involved in cholesterol trafficking is associated with increased cellularity and less apoptosis in advanced atherosclerotic lesions. These findings highlight important issues related to mechanisms, consequences, and potential therapeutic implications of FC-induced macrophage death during the progression of atherosclerosis. Regarding the potential for therapy, we have shown that very low doses of cholesterol-trafficking inhibitor U18666A selectively inhibit cholesterol trafficking to the ER and block UPR activation and apoptosis in FC-loaded cultured macrophages almost completely (22). Furthermore, the atherosclerosis data raise the interesting question whether heterozygous Niemann-Pick C humans, who are reported to be “normal” (35), actually have a lower incidence of acute ischemic events compared with individuals without this mutation.

References for Fourth Series of Experiments

1. Ross, R. (1995) Annu. Rev. Physiol. 57, 791-804.

2. Libby, P. & Clinton, S. K. (1993) Curr. Opin. Lipidol. 4, 355-363.

3. Mitchinson, M. J., Hardwick, S. J. & Bennett, M. R. (1996) Curr. Opin. Lipidol. 7, 324-329.

4. Kockx, M. M. (1998) Arterioscler. Thromb. Vasc. Biol. 18, 1519-1522.

5. Berberian, P. A., Myers, W., Tytell, M., Challa, V. & Bond, M. G. (1990) Am. J. Pathol. 136, 71-80. 06. Ball, R. Y., Stowers, E. C., Burton, J. H., Cary, N. R., Skepper, J. N. & Mitchinson, M. J. (1995) Atherosclerosis (Shannon, Irel.) 114, 45-54.

7. Fuster, V., Badimon, L., Badimon, J. J. & Chesebro, J. H. (1992) N. Engl. J. Med. 326, 242-250.

8. Kolodgie, F. D., Narula, J., Burke, A. P., Haider, N., Farb, A., Hui-Liang, Y., Smialek, J. & Virmani, R. (2000) Am. J. Pathol. 157, 1259-1268.

9. Tabas, I. (1997) Trends Cardiovasc. Med. 7, 256-263.

10. Shio, H., Haley, N.J. & Fowler, S. (1979) Lab. Invest. 41, 160-167.

11. Rapp, J. H., Connor, W. E., Lin, D. S., Inahara, T. & Porter, J. M. (1983) J. Lipid Res. 24, 1329-1335.

12. Small, D. M., Bond, M. G., Waugh, D., Prack, M. & Sawyer, J. K. (1984) J. Clin. Invest. 73, 1590-1605.

13. Kruth, H. S. & Fry, D. L. (1984) Exp. Mol. Pathol. 40, 288-294.

14. Lundberg, B. (1985) Atherosclerosis (Shannon, Irel.) 56, 93-110.

15. Warner, G. J., Stoudt, G., Bamberger, M., Johnson, W. J. & Rothblat, G. H. (1995) J. Biol. Chem. 270, 5772-5778.

16. Tabas, I., Marathe, S., Keesler, G. A., Beatini, N. & Shiratori, Y. (1996) J. Biol. Chem. 271, 22773-22781.

17. Yao, P. M. & Tabas, I. (2000) J. Biol. Chem. 275, 23807-23813.

18. Yao, P. M. & Tabas, I. (2001) J. Biol. Chem. 276, 42468-42476.

19. Fazio, S., Major, A. S., Swift, L. L., Gleaves, L. A., Accad, M., Linton, M. F. & Farese, R. V., Jr. (2001) J. Clin. Invest. 107, 163-171.

20. Chang, M.-K., Bergmark, C., Laurila, A., Horkko, S., Han, K.-H., Friedman, P., Dennis, E. A. & Witztum, J. L. (1999) Proc. Natl. Acad. Sci. USA 96, 6353-6358.

21. Lemasters, J. J. (1999) Am. J. Physiol. 276, G1-G6.

22. Feng, B. Yao, P. M., Li, Y., Devlin, C. M., Zhang, D., Harding, H. P., Sweeney, M., Rong, J. X., Kuriakose, G., Fisher, E. A., et al. (2003) Nat. Cell Biol., in press.

23. Pentchev, P. G., Boothe, A. D., Kruth, H. S., Weintroub, H., Stivers, J. & Brady, R. O. (1984) J. Biol. Chem. 259, 5784-5791.

24. Loftus, S. K., Morris, J. A., Carstea, E. D., Gu, J. Z., Cummings, C., Brown, A., Ellison, J., Ohno, K., Rosenfeld, M. A., Tagle, D. A., et al. (1997) Science 277, 232-235.

25. Liscum, L. & Klansek, J. J. (1998) Curr. Opin. Lipidol. 9, 131-135.

26. Pentchev, P. G., Blanchette-Mackie, E. J. & Liscum) L. (1997) Subcell. Biochem. 28, 437-451.

27. Kockx, M. M., Muhring, J., Knaapen, M. W. & De Meyer, G. R. (1998) Am. J. Pathol. 152, 885-888.

28. Devlin, C. M., Kuriakose, G., Hirsch, E. & Tabas, I. (2002) Proc. Natl. Acad. Sci. USA 99, 6280-6285.

29. Nakashima, Y., Plump, A. S., Raines, E. W., Breslow, J. L. & Ross, R. (1994) Arterioscler. Thromb. 14, 133-140.

30. Reddick, R. L., Zhang, S. H. & Maeda, N. (1994) Arterioscler. Thromb. 14, 141-147.

31. Kruth, H. S. (1985) Atherosclerosis (Shannon, Irel.) 57, 337-341.

32. Guyton, J. R. & Klemp, K. F. (1996) Arterioscler. Thromb. Vasc. Biol. 16, 4-11.

33. Smith, J. D., Trogan, E., Ginsberg, M., Grigaux, C., Tian, J. & Miyata, M. (1995) Proc. Natl. Acad. Sci. USA 92, 8264-8268.

34. Schaefer, H. E. (1981) in Disorders of the Monocyte-Macrophage System, eds. Schmalze, F., Huhn, D. & Schaefer, H. E. (Springer, Berlin), pp. 137-142.

35. Pentchev, P. G., Vanier, M. T., Suzuki, K. & Patterson, M. C. (1995) in Metabolic Basis of Inherited Disease, eds. Scriver, C. R., Beaudet, A. L., Sly, W. S. & Valle, D. (McGraw-Hill, New York), pp. 2625-2639.

36. Feng, B. et al. (2003) PNAS 100(18): 10423-10428.

1. A method for inhibiting macrophage death in a subject having, or at increased risk for developing, cardiovascular disease which comprises administering to the subject an effective amount of an amphiphilic compound or a pharmaceutically acceptable salt thereof which inhibits the intracellular transport of cholesterol within cells, wherein the transport is from an intracellular cholesterol storage site to the endoplasmic reticulum, so as to thereby inhibit macrophage death in the subject.

2. A method for inhibiting atherosclerotic lesional complications in a subject having, or at increased risk for developing, cardiovascular disease which comprises administering to the subject an effective amount of an amphiphilic compound or a pharmaceutically acceptable salt thereof which inhibits the intracellular transport of cholesterol within cells, wherein the transport is from an intracellular cholesterol storage site to the endoplasmic reticulum, so as to thereby inhibit atherosclerotic lesional complications in the subject.

3.-12. (canceled)

13. A method for inhibiting macrophage death in a subject having, or at increased risk for developing, cardiovascular disease which comprises administering to the subject an effective amount of an amphiphilic compound or a pharmaceutically acceptable salt thereof which inhibits free cholesterol-induced death of cells in the subject by inhibiting intracellular transport of cholesterol within the cells, wherein the transport is from an intracellular cholesterol storage site to the endoplasmic reticulum, so as to thereby inhibit macrophage death in the subject.

14. A method for inhibiting atherosclerotic lesional complications in a subject having, or at increased risk for developing, cardiovascular disease which comprises administering to the subject an effective amount of an amphiphilic compound or a pharmaceutically acceptable salt thereof which inhibits free cholesterol-induced death of cells in the subject by inhibiting intracellular transport of cholesterol within the cells, wherein the transport is from an intracellular cholesterol storage site to the endoplasmic reticulum, so as to thereby inhibit atherosclerotic lesional complications in the subject.

15.-26. (canceled)

27. A method for inhibiting necrosis, plaque rupture and/or superficial erosion in a subject having, or at increased risk for developing, cardiovascular disease which comprises administering to the subject an effective amount of an amphiphilic compound or a pharmaceutically acceptable salt thereof which inhibits intracellular transport of cholesterol within cells, wherein the transport is from an intracellular cholesterol storage site to the endoplasmic reticulum, so as to thereby inhibit necrosis, plaque rupture and/or superficial erosion in the subject.
